(19) United States
(12) Patent Application Publication
(10) Pub. No.: US 2024/0383955 A1
(43) Pub. Date: Nov. 21, 2024

(54) MODIFIED ANTIMICROBIAL PEPTIDES

(71) Applicant: Donald Danforth Plant Science Center, ST. LOUIS, MO (US)

(72) Inventors: Dilip M. Shah, St. Louis, MO (US); Meenakshi Tetorya, St. Louis, MO (US)

(21) Appl. No.: 18/569,765

(22) PCT Filed: Jun. 16, 2022
(86) PCT No.: PCT/US2022/072971

§ 371 Date: Dec. 13, 2023

Related U.S. Application Data
(60) Provisional application No. 63/365,928, filed on Jun. 6, 2022.
(60) Provisional application No. 63/202,559, filed on Jun. 16, 2021.

(51) Int. Cl. C07K 14/47; A01H 5/10; A01N 37/46; A01P 1/00; A01P 3/00; A61K 38/00; A61P 31/10; C12N 15/82

(52) U.S. Cl. CPC C07K 14/4723; A01H 5/10; A01N 37/46; A01P 1/00; A01P 3/00; A61P 31/10; C12N 15/8282; A61K 38/00

(57) ABSTRACT

Antimicrobial defensin peptide variants comprising modified C-terminal fragments of a defensin and nucleic acids encoding the same are disclosed. Compositions comprising the defensin variant peptides and methods of their use to control microbial infections of plants and vertebrate subjects as well as contamination of feedstuffs and foodstuffs are also disclosed.

### CROSS-REFERENCE TO RELATED APPLICATIONS

This application claims the benefit of U.S. Patent Application Ser. No. 63/365,928, filed Jun. 6, 2022 and U.S. Patent Application Ser. No. 63/202,559, filed Jun. 16, 2021, which are each incorporated herein by reference in their entireties.

### GOVERNMENT SUPPORT STATEMENT

This invention was made with government support under National Science Foundation EAGER Award Number 1955461 awarded by the National Science Foundation. The government has certain rights in the invention.

### SEQUENCE LISTING

The sequence listing contained in the file named “P13511WO00_ST25”, which is 294,870 bytes as measured in the Windows operating system, and which was created on Jun. 15, 2022, and electronically filed herewith, is incorporated herein by reference in its entirety.

### BACKGROUND

Protection of agriculturally important crops from pathogenic microbes (e.g., fungi or oomycetes) is crucial in improving crop yields. Microbial infections are a particular problem in damp climates and can become a major concern during crop storage, where such infections can result in spoilage and contamination of food or feed products with microbial toxins. Unfortunately, modern growing methods, harvesting and storage systems can promote plant pathogen infections.

Control of plant pathogens is further complicated by the need to simultaneously control multiple microbes of distinct genera. For example, microbes such as Alternaria; Ascochyta; Aphenomyces; Botrytis; Cercospora; Colletotrichum; Diplodia; Erysiphe; Fusarium; Gaeumanomyces; Helminthosporium; Leptosphaeria, Macrophomina; Magnaporthe; Nectria; Peronospora; Phoma; Phakopsora, Phymatotrichum; Phytophthora; Plasmopara; Podosphaera; Puccinia; Pythium; Pyrenophora; Pyricularia; Rhizoctonia; Sclerotium; Sclerotinia; Septoria; Thielaviopsis; Uncinula; Venturia; and Verticillium species are all recognized plant pathogens.

Certain microbes (e.g., fungi, including mold, yeast and dimorphic fungi, or oomycetes) can also be pathogenic to various vertebrates including humans, livestock, companion animals, fish, and the like. Microbes including dermatophytes, Aspergillus, Candida, Cryptococcus, Coccidiomyces, Penicillium, Rhizopus, Apophysomyces, Cunninghamella, Saksenaea, Rhizomucor, Syncephalostrum, Cokeromyces, Actinomucor, Pythium, Fusarium, Histoplasmosis, or Blastomyces species are also important vertebrate pathogens.

A group of proteins known as defensins have been shown to inhibit plant pathogens. Defensins have been previously identified as small cysteine-rich peptides of about 45-54 amino acids that constitute an important component of the innate immunity of plants (Thomma et al., 2002; Lay and Anderson, 2005; Vriens et al., 2014). Widely distributed in plants, defensins vary greatly in their amino acid composition. However, they all have a compact shape which is stabilized by either four or five intramolecular disulfide bonds. Plant defensins have previously been characterized as comprising a conserved gamma core peptide comprising a conserved GXCX3-9C (where X is any amino acid) sequence (Sagaram et al., 2011; Lacerda et al., 2014). The three dimensional structure of the previously characterized gamma core peptide consists of two antiparallel β-sheets, with an interpolated turn region (Ibid.). Antimicrobial activity of certain defensins has been correlated with the presence of positively charged amino acid residues in the gamma core peptide (Spelbrink et al, Plant Physiol., 2004; Sagaram et al, 2013).

Plant defensins have been extensively studied for their role in plant defense. Some plant defensins inhibit the growth of a broad range of microbes at micromolar concentrations (Broekaert et al, 1995; Broekaert et al, 1997; da Silva Conceicao and Broekaert, 1999) and, when expressed in transgenic plants, confer strong resistance to microbial pathogens (da Silva Conceicao and Broekaert, 1999; Thomma et al., 2002; Lay and Anderson, 2005). Two small cysteine-rich proteins isolated from radish seed, Rs-AFP1 and Rs-AFP2, inhibited the growth of many pathogenic microbes when the pure protein was added to an in vitro antimicrobial assay medium (U.S. Pat. No. 5,538,525). Transgenic tobacco plants containing the gene encoding Rs-AFP2 protein were found to be more resistant to attack by microbes than non-transformed plants.

Defensin genes have also been identified in the legume Medicago truncatula (Hanks et al., 2005). The cloned MtDef2 protein has been demonstrated through in vitro experiments to have little or no antimicrobial activity (Spelbrink et al., 2004). The Medicago truncatula defensin proteins MtDef4 (U.S. Pat. No. 7,825,297; incorporated herein by reference in its entirety) and MtDef5 (WO2014179260 and US Patent Appl. Pub. No. 20160208278; both incorporated herein by reference in its entirety) have antimicrobial activity. The C-terminal 16 amino acid GMA4-C peptide of the MtDef4 defensin protein inhibited Fusarium graminearum at concentrations as low as 3 μM (Sagaram et al., 2011)

Plant defensins with potent antifungal activity in vitro often fail to confer effective disease resistance in planta. This constrains their commercial development as antifungal agents in transgenic crops. Antifungal plant defensins are generally cationic and cationic residues in their sequences are believed to initiate passage through fungal cell envelope by electrostatic interactions with the anionic fungal cell membrane (Kerenga et al., 2019). Potassium (K+) is an essential macronutrient and is also the most abundant cation in plants. The concentration of K+ in the plant cell cytoplasm is consistently between 100 and 200 mM (Shabala and Pottosin, 2010 and between 10 and 200 mM in the apoplast (White and Karley, 2010). Calcium is an essential secondary micronutrient and its concentrations can range from 0.1% to 6% of the dry weight of plants (Broadley et al., 2003). The concentrations of sodium (Na+) in plants range from 0.001%-8% (Marschner, 1995). Na+ is an essential micronutrient for plants in saline soils.

Many plant defensins that have been characterized to date lose their antifungal activity at elevated concentrations of mono- and bivalent cations such as 100 mM KCl or 2 mM CaCl₂). However, the maize plant defensin ZmD32 having a predicted charge of +10.1 at pH 7 exhibits inhibitory activity against Candida sp. and E. coli in the presence of 100 mM NaCl while the Nicotiana benthamiana plant defensin NbD6 having a predicted charge of +7.6 at pH 7 exhibits inhibitory activity against Candida albicans in the presence of 100 mM NaCl (Kerenga et al., 2019).

### SUMMARY

A peptide comprising the sequence XGXCXGFXXXX(F/W/Y)XXXXC (SEQ ID NO: 1), wherein said peptide does not comprise the corresponding full-length sequence of the defensin peptide of SEQ ID NO: 8, 16, 20, 23, 26, 29, 37, 38, 39, 40, or 41; optionally wherein said peptide comprises a modified gamma-core consensus sequence GXCX3-8 (F/W/Y)(F/W/Y)(F/W/Y) set forth in SEQ ID NO: 33, GXCX3-9(F/W/Y)(F/W/Y)(F/W/Y) set forth in SEQ ID NO: 34, GXCX3-8 (F/W/Y) (SEQ ID NO: 43), or GXCX3-9(F/W/Y) (SEQ ID NO: 44); and/or optionally wherein the C-terminal cysteine residue or C-terminal amino acid residue is amidated is provided. A peptide having at least 75%, 82%, or 94% sequence identity across the entire length of any one of SEQ ID NO: 3, 4, 5, or 6, wherein the peptide is not identical to SEQ ID NO: 8, optionally wherein any amino acid substitution in said sequence increases or maintains the net positive charge at neutral pH and/or increases or maintains hydrophobicity of the peptide, and optionally wherein the C-terminal cysteine residue is amidated. A peptide having at least 75%, 82%, or 94% sequence identity across the entire length of SEQ ID NO:7, 12, 13, 14, 15, 17-20, 22, 23, 25, 26, 28, 29, 31, and 32, wherein the peptide is not identical to SEQ ID NO: 8, 16, 20, 23, 26, 29, 37, 38, 39, 40, or 41, optionally wherein the peptide of SEQ ID NO:5, SEQ ID NO: 13, 14, 15, 18, 19, 20, 22, 23, 26, 28, 29, 31, or 32 comprises a disulfide bond between the two cysteine residues and optionally wherein any substitution increases or maintains the net positive charge at neutral pH and/or increases or maintains hydrophobicity of the peptide, and optionally wherein the C-terminal cysteine residue or C-terminal amino acid residue is amidated, is provided. A defensin C-terminal peptide variant comprising conserved C1 and C4 cysteine residues corresponding to N-terminal and C-terminal cysteines of a reference defensin C-terminal peptide, wherein conserved C2 and C3 cysteine residues of the reference defensin C-terminal peptide are independently substituted with tryptophan, tyrosine, phenylalanine, leucine, valine, isoleucine, or methionine; and optionally wherein the defensin peptide variant has a net positive charge of at least 3, 3.5, 4, 5, or 6 and a hydrophobic amino acid content at least 18% is provided. A C-terminal defensin peptide variant comprising a modified gamma-core consensus sequence GXCX3-8 (F/W/Y)(F/W/Y)(F/W/Y) (SEQ ID NO: 33), GXCX3-9(F/W/Y)(F/W/Y)(F/W/Y) (SEQ ID NO: 34), GXCX3-8 (F/W/Y) (SEQ ID NO: 43), GXCX3-9(F/W/Y) (SEQ ID NO: 44), GXCX3-8(F/W/Y/L/V/I/M) (SEQ ID NO: 45), GXCX3-10(F/W/Y/L/V/I/M) (SEQ ID NO: 46), GXCX3-8(L/V/I/M)(F/W/Y)(L/V/I/M) (SEQ ID NO: 47), GXCX3-10(L/V/I/M)(F/W/Y)(L/V/I/M) (SEQ ID NO: 48), GXCX3-8(F/W/Y/L/V/I/M) (F/W/Y)(F/W/Y/L/V/I/M) (SEQ ID NO: 49), GXCX3-10(F/W/Y/L/V/I/M) (F/W/Y)(F/W/Y/L/V/I/M) (SEQ ID NO: 50), GXCX3-8(F/W/Y/L/V/I/M)(F/W/Y/L/V/I/M)(F/W/Y/L/V/I/M) (SEQ ID NO: 51), GXCX3-10(F/W/Y)(F/W/Y)(F/W/Y) (SEQ ID NO: 587), GXCX3-12(F/W/Y)(F/W/Y)(F/W/Y) (SEQ ID NO: 588), GXCX3-15(F/W/Y)(F/W/Y)(F/W/Y) ((SEQ ID NO: 589), or GXCX3-10(F/W/Y/L/V/I/M)(F/W/Y/L/V/I/M)(F/W/Y/L/V/I/M) (SEQ ID NO: 52) is provided. In certain embodiments, the aforementioned peptide exhibits antimicrobial activity, wherein the antimicrobial activity is optionally one or more of an antifungal or antibacterial activity. Compositions comprising any of the aforementioned peptides and an agriculturally, pharmaceutically, or veterinary-practicably acceptable carrier, diluent, or excipient are also provided. Compositions comprising any of the aforementioned peptides and carriers, diluents, or excipients for use in treating, preventing, or inhibiting microbial infection in a subject in need thereof are also provided.

Methods for: (i) preventing or reducing crop damage by a plant pathogenic microbe or (ii) preventing contamination of plants, plant parts, seeds, feedstuff obtained therefrom, or foodstuff obtained therefrom with an undesirable microbe, comprising the step of contacting a plant, a plant seed, or other part of said plant with an effective amount of any of the aforementioned compositions, where the composition optionally comprises an agriculturally acceptable carrier, diluent, or excipient, are also provided.

Plant parts including seeds which are least partly coated with any of the aforementioned compositions, where the composition optionally comprises an agriculturally acceptable carrier, diluent, or excipient, are also provided.

Medical devices comprising the device and an aforementioned composition, wherein the device comprises at least one surface that is topically coated and/or impregnated with the composition, where the composition optionally comprises a pharmaceutically or veterinary-practicably acceptable carrier, diluent, or excipient are also provided.

Methods for treating, preventing, or inhibiting a microbial infection in a subject in need thereof comprising administering to said subject an effective amount of any of the aforementioned compositions, where the composition optionally comprises a pharmaceutically or veterinary-practicably acceptable carrier, diluent, or excipient are also provided. Use of any of any of the aforementioned compositions in a method of treating, preventing, or inhibiting microbial or yeast infection in a subject in need thereof are provided. Use of any of the aforementioned first antimicrobial peptide or proteins in the manufacture of a medicament or composition for inhibiting microbial or yeast infection in a subject in need thereof are also provided.

Recombinant polynucleotides comprising a polynucleotide peptides comprising any of the aforementioned peptides, wherein the polynucleotide encoding the antimicrobial peptide is operably linked to a polynucleotide comprising a promoter which is heterologous to the polynucleotide encoding the first antimicrobial peptide, optionally wherein any amino acid substitution in said sequence increases or maintains the net positive charge and/or hydrophobicity of the peptide, are provided.

Plant nuclear or plastid genomes comprising a polynucleotide encoding an antimicrobial peptide comprising any of the aforementioned peptides, wherein the polynucleotide is heterologous to the nuclear or plastid genome and wherein the polynucleotide is operably linked to an endogenous promoter of the nuclear or plastid genome, are provided.

Cells, plant, and plant parts including seeds comprising the aforementioned recombinant polynucleotides or genomes are provided.

### BRIEF DESCRIPTION OF THE DRAWINGS

FIGS. 1A and 1B show drop inoculation assays on Nicotiana benthamiana leaves. Four-week-old leaves of N. benthamiana were used for drop-inoculation assays against B. cinerea with different concentration of GMA4C peptides. 1A. Pictorial representation of the drop-inoculation assays and pictures were taken after 48 h of incubation in both white-light and with CropReporter. Damaged area is represented in red color in CropReporter images. Data for GMA4C_AC wild-type control (SEQ ID NO: 2) is in left most panel (labelled “GMA4C”), data for GMA4c_ViA (SEQ ID NO:3) is in panel 2^{nd} from left, data for GMA4c_V2A (SEQ ID NO:4) is in panel 3rd from left, and data for GMA4c_V3A (SEQ ID NO:5) is in panel 4th from left. 1B, Lesions were measured after 48 h using the ImageJ software and their sizes are shown on the graph.

FIG. 2 shows an alignment of a non-limiting subset of reference defensin C-terminal peptides which include the conserved C1, C2, C3, and C4 cysteines (in bold). The conserved gamma core peptide is underlined.

FIG. 3 shows an alignment of a non-limiting subset of reference defensin C-terminal peptides which include the conserved C1, C2, C3, and C4 cysteines (in bold), the gamma-core consensus, and various modified gamma-core consensus sequences provided herein. The conserved gamma core peptide is underlined and is absent from the modified gamma-core consensus peptides.

FIG. 4 shows a data table with antimicrobial activity of various GMA4C defensin peptide variants.

FIG. 5A, B. FIG. 5A, B show results of a drop inoculation assay showing the antifungal activity of GMA4C_V9 (SEQ ID NO: 581) and GMA4C_V10 peptides (SEQ ID NO: 582). GMA4C_V10 completely abrogates gray mold symptoms at 6 μM. In FIG. 5B, the left-most bars are for the no treatment control (“B. cinerea”), middle bars are GMA4C_V9 (SEQ ID NO: 581) treatment, and rightmost bars are GMA4C_V10 (SEQ ID NO: 582) treatment. At 3 μM and 1.5 μM, GMA4C_V10 is more effective in reducing the symptoms of gray mold than GMA4C_V9.

FIG. 6A, B show results of a drop inoculation assay showing the antifungal activity of GMAOe1C_WT (SEQ ID NO: 577), GMAOe1C_V3 (SEQ ID NO: 578), and GMAOe1C_V4 (SEQ ID NO: 579). In FIG. 6B, the left-most bars are for the no treatment control (“B. cinerea”), 2^{nd} from left bars are GMAOe1C_WT treatment, 3^{rd} from left bars are GMAOe1C_V3 treatment, and 4^{th} from left bars are GMAOe1C_V4 treatment. GMAOe1C_V3 and GMAOe1C_V4 at 3 μM and 6 μM completely abrogated gray mold symptoms but GMAOe1C_WT is only effective at 6 μM. At a concentration of 1.5 μM, GMAOe1C_V3 is more effective than GMAOe1C_V4 or GMAOe1C_WT.

FIG. 7A, B show results of a drop inoculation assay showing the antifungal activity of GMA1C_V1 (SEQ ID NO: 583) and GMA1C_V2 (SEQ ID NO: 584) against B. cinerea. In FIG. 7B, the left-most bars are for the no treatment control (“B. cinerea”), middle bars are GMA1C_V1 (SEQ ID NO: 583) treatment, and rightmost bars are GMA1C_V2 (SEQ ID NO: 584) treatment.

### DETAILED DESCRIPTION

### Definitions

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

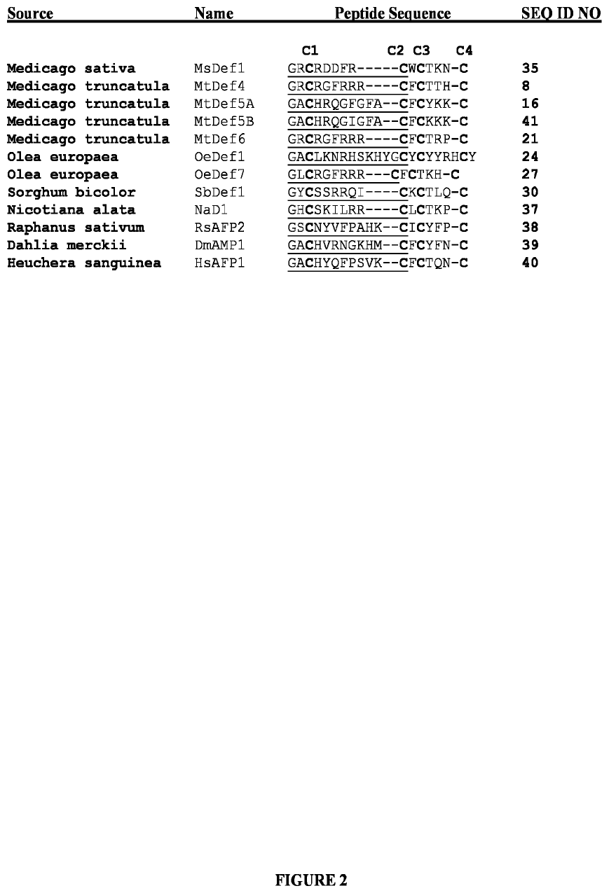

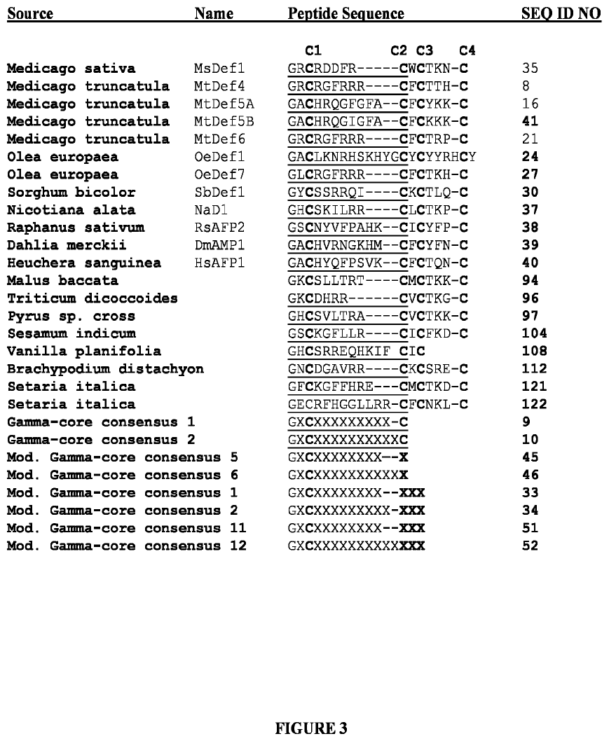

As used herein, the terms “correspond,” “corresponding,” and the like, when used in the context of an amino acid position, mutation, and/or substitution in any given peptide (e.g., a defensin variant peptide) with respect to the reference peptide sequence (e.g., reference defensin C-terminal peptide sequence including SEQ ID NO: 8, 16, 20, 23, 26, 29, 37, 38, 39, 40, 41 or 89-122) all refer to the amino acid residue in the given peptide sequence that has the same location in the given peptide as the residue in the reference amino acid sequence when the given peptide is aligned to the reference sequence. In certain embodiments, the alignment is an alignment of the 4 conserved cysteine residues of a defensin C-terminal peptide of a defensin variant peptide and a reference defensin C-terminal peptide sequence (e.g., as shown in FIGS. 2 and 3).

As used herein, the terms “include,” “includes,” and “including” are to be construed as at least having the features to which they refer while not excluding any additional unspecified features.

Where a term is provided in the singular, other embodiments described by the plural of that term are also provided.

The phrase “antimicrobial peptide” as used herein refer to peptides which exhibit any one or more of the following characteristics of inhibiting the growth of microbial cells, killing microbial cells, disrupting or retarding stages of the microbial life cycle such as spore germination, sporulation, or mating, and/or disrupting microbial cell infection, penetration or spread within a plant or other susceptible subject, including a human, livestock, poultry, fish, or a companion animal (e.g. dog or cat).

As used herein, the terms “acidic” or “anionic” are used interchangeably to refer to amino acids such as aspartic acid and glutamic acid.

As used herein, the term “amino acid” refers to an organic compound that contains amino (—NH₃) and carboxylate (—CO₂) functional groups, along with a side chain (R group) specific to each amino acid. Amino acid residues in polypeptides are in certain instance referred to herein by one letter amino acid codes as follows: G—Glycine (Gly); P—Proline (Pro); A—Alanine (Ala); V—Valine (Val); L—Leucine (Leu); I—Isoleucine (Ile); M—Methionine (Met); C—Cysteine (Cys); F—Phenylalanine (Phe); Y—Tyrosine (Tyr); W—Tryptophan (Trp); H—Histidine (His); K—Lysine (Lys); R—Arginine (Arg); Q—Glutamine (Gln); N—Asparagine (Asn); E—Glutamic Acid (Glu); D—Aspartic Acid (Asp); S—Serine (Ser); or T—Threonine (Thr).

As used herein, the terms “basic” and “cationic” are used interchangeably to refer to amino acids such as arginine, histidine, and lysine.

As used herein, the phrase “cation-tolerant” refers to a defensin peptide or defensin peptide variant which exhibits equivalent in vitro antifungal or antimicrobial activity or no more than about a 1.5-, 2-, 3-, or 4-fold decrease in in vitro antifungal or antimicrobial activity in the presence of 100 mM KCl or 100 mM NaCl as compared to the antifungal activity of the defensin peptide or defensin peptide variant in the absence of KCl or NaCl.

As used herein, the phrase “consensus sequence” refers to an amino acid, DNA or RNA sequence created by aligning two or more homologous sequences and deriving a new sequence having either the conserved or set of alternative amino acid, deoxyribonucleic acid, or ribonucleic acid residues of the homologous sequences at each position in the created sequence.

The phrases “combating microbial damage”, “combating or controlling microbial damage” or “controlling microbial damage” as used herein refer to reduction in damage to a crop plant or crop plant product due to infection by a microbial pathogen. More generally, these phrases refer to reduction in the adverse effects caused by the presence of a pathogenic microbe in the crop plant. Adverse effects of microbial growth are understood to include any type of plant tissue damage or necrosis, any type of plant yield reduction, any reduction in the value of the crop plant product, and/or production of undesirable microbial metabolites or microbial growth by-products including to mycotoxins.

The phrase “defensin peptide” is used herein to refer to a peptide comprising a conserved gamma core peptide. Plant defensins have been previously characterized as comprising a conserved GXCX3-9C gamma core peptide sequence (SEQ ID NO: 9), where X is any amino acid residue (Lacerda et al.) or a conserved GXCX3-10C variant gamma core peptide sequence (SEQ ID NO: 10), where X is any amino acid residue. In certain embodiments, defensin peptides disclosed herein can also include non-standard defensin gamma core peptides comprising a GXCX3-12C (SEQ ID NO: 590) or GXCX3-15C (SEQ ID NO: 591). Therefore, as used in this disclosure, a plant defensin or defensin or C-terminal peptide comprising fragment thereof can comprise a conserved GXCX3-9C (SEQ ID NO: 9), GXCX3-10C (SEQ ID NO: 10), GXCX3-12C (SEQ ID NO: 590) or GXCX3-15C (SEQ ID NO: 591) gamma core peptide sequence, where X is any amino acid residue. Defensin peptides include proteins that are antimicrobial, that can permeabilize plasma membranes, that can bind phospholipids, that can bind sphingolipids, or that exhibit any combination of those properties. A defensin peptide can be naturally occurring or non-naturally occurring (e.g., synthetic and/or chimeric).

The phrase “defensin peptide variant” is used herein to describe a modified defensin peptide comprising either: (i) a conserved gamma core peptide of SEQ ID NO: 9 or 10 and at least one amino acid substitution in a source defensin peptide; or (ii) a modified gamma core variant sequence GXCX3-8(F/W/Y)(F/W/Y)(F/W/Y) (SEQ ID NO: 33), GXCX3-9(F/W/Y)(F/W/Y)(F/W/Y) (SEQ ID NO: 34), GXCX3-8 (F/W/Y) (SEQ ID NO: 43), GXCX3-9(F/W/Y) (SEQ ID NO: 44), GXCX3-8(F/W/Y/L/V/I/M) (SEQ ID NO: 45), GXCX3-10(F/W/Y/L/V/I/M) (SEQ ID NO: 46), GXCX3-8(L/V/I/M)(F/W/Y)(L/V/I/M) (SEQ ID NO: 47), GXCX3-10(L/V/I/M)(F/W/Y)(L/V/I/M) (SEQ ID NO: 48), GXCX3-8(F/W/Y/L/V/I/M) (F/W/Y)(F/W/Y/L/V/I/M) (SEQ ID NO: 49), GXCX3-10(F/W/Y/L/V/I/M) (F/W/Y)(F/W/Y/L/V/I/M) (SEQ ID NO: 50), GXCX3-8(F/W/Y/L/V/I/M)(F/W/Y/L/V/I/M)(F/W/Y/L/V/I/M) (SEQ ID NO: 51), GXCX3-10(F/W/Y)(F/W/Y)(F/W/Y) (SEQ ID NO: 587), GXCX3-12(F/W/Y)(F/W/Y)(F/W/Y) (SEQ ID NO: 588), GXCX3-15(F/W/Y)(F/W/Y)(F/W/Y) ((SEQ ID NO: 589), or GXCX3-10(F/W/Y/L/V/I/M)(F/W/Y/L/V/I/M)(F/W/Y/L/V/I/M) (SEQ ID NO: 52). In certain embodiments, defensin peptide variants provided herein are less than full length defensin peptides (e.g., peptides comprising, consisting essentially of, or consisting of: (i) 30 amino acid residues or less; or (ii) 15, 16, or 17 to 30 amino acid residues).

The phrase “reference defensin C-terminal peptide” is used herein to refer to a less than full length defensin peptide comprising a conserved GXCX3-9C gamma core peptide sequence (SEQ ID NO: 9), a conserved GXCX3-10C variant gamma core peptide sequence (SEQ ID NO: 10), a non-standard GXCX3-12C (SEQ ID NO: 590) gamma core sequence, or a non-standard GXCX3-15C (SEQ ID NO: 591) gamma core sequence, and the two additional conserved cysteine residues located C-terminal to the gamma core peptide sequence, wherein the cysteine located closest to the N-terminus of the reference defensin C-terminal peptide corresponds to the cysteine located closest to the N-terminus of the gamma core sequence of SEQ ID NO: 9 or SEQ ID NO: 10. Examples of reference defensin C-terminal peptides include an MtDef4 C-terminal peptide (SEQ ID NO: 8), an MtDef5A C-terminal peptide (SEQ ID NO: 16), an MtDef5B C-terminal peptide (SEQ ID NO: 41), an MtDef6 peptide (SEQ ID NO: 21), an OeDef1 C-terminal peptide (SEQ ID NO: 24), an OeDef7 C-terminal peptide (SEQ ID NO: 27), an SbDef1 C-terminal peptide (SEQ ID NO: 30), an NaD1 C-terminal peptide (SEQ ID NO: 37), an RsAFP2 C-terminal peptide (SEQ ID NO: 38), a DmAMP1 C-terminal peptide (SEQ ID NO: 39), and an HsAFP1 (SEQ ID NO: 40). Other examples of reference defensin C-terminal peptides include SEQ ID NO: 89 to 122. Reference defensin C-terminal peptides thus include 4 conserved cysteine residues, which are referred to herein as C1 (most N-terminal in both the peptide and in the gamma core peptide sequence), C2 (C-terminal to C1 and located in the gamma core peptide sequence), C3 (C-terminal to C2), and C4 (C-terminal to C3). An alignment of a non-limiting subset of reference defensin C-terminal peptides comprising the conserved gamma core peptide sequence and the C1, C2, C3, and C4 cysteines is shown in FIG. 2. An alignment of a non-limiting subset of reference defensin C-terminal peptides comprising the conserved gamma core peptide sequence and the C1, C2, C3, and C4 cysteines, the gamma core consensus comprising the C1 and C2 cysteines, and certain modified gamma core consensus sequences provided herein is shown in FIG. 3. Other reference defensin C-terminal peptides that include those contained in the full length defensin peptides of SEQ ID NO: 123 to 434 and 436 to 576.

As used herein, the terms “edit,” “editing,” “edited” and the like refer to processes or products where insertions, deletions, and/or nucleotide substitutions are introduced into a genome. Such processes include methods of inducing homology directed repair and/or non-homologous end joining of one or more sites in the genome.

The phrases “genetically edited plant” or “edited plant” are used herein to refer to a plant comprising one or more nucleotide insertions, deletions, substitutions, or any combination thereof in the genomic DNA of the plant. Such genetically edited plants can be constructed by techniques including CRISPR/Cas endonuclease-mediated editing, meganuclease-mediated editing, engineered zinc finger endonuclease-mediated editing, and the like.

The term “heterologous”, as used herein in the context of a second polynucleotide that is operably linked to a first polynucleotide, refers to: (i) a second polynucleotide that is derived from a source distinct from the source of the first polynucleotide; (ii) a second polynucleotide derived the same source as the first polynucleotide, where the first, second, or both polynucleotide sequence(s) is/are modified from its/their original form; (iii) a second polynucleotide arranged in an order and/or orientation or in a genomic position or environment with respect to the first polynucleotide that is different than the order and/or orientation in or genomic position or environment of the first and second polynucleotides in a naturally occurring cell; or (iv) the second polynucleotide does not occur in a naturally occurring cell that contains the first polynucleotide. Heterologous polynucleotides include polynucleotides that promote transcription (e.g., promoters and enhancer elements), transcript abundance (e.g., introns, 5′UTR, and 3′UTR), translation, or a combination thereof as well as polynucleotides encoding defensin peptide variants or defensin peptides, spacer peptides, or localization peptides. In certain embodiments, a nuclear or plastid genome can comprise the first polynucleotide, where the second polynucleotide is heterologous to the nuclear or plastid genome. A “heterologous” polynucleotide that promotes transcription, transcript abundance, translation, or a combination thereof as well as polynucleotides encoding defensin peptide variants or defensin peptides, spacer peptides, or localization peptides can be autologous to the cell but, however, arranged in an order and/or orientation or in a genomic position or environment that is different than the order and/or orientation in or genomic position or environment in a naturally occurring cell. A polynucleotide that promotes transcription, transcript abundance, translation, or a combination thereof as well as polynucleotides encoding defensin peptide variants or defensin peptides, spacer peptides, or localization can be heterologous to another polynucleotide when the polynucleotides are not operably linked to one another in a naturally occurring cell. Heterologous peptides or proteins include peptides or proteins that are not found in a cell or organism as the cell or organism occurs in nature. As such, heterologous peptides or proteins include peptides or proteins that are localized in a subcellular location, extracellular location, or expressed in a tissue that is distinct from the subcellular location, extracellular location, or tissue where the peptide is found in a cell or organism as it occurs in nature. Heterologous polynucleotides include polynucleotides that are not found in a cell or organism as the cell or organism occurs in nature.

The phrases “inhibiting growth of a plant pathogenic microbe,” “inhibit microbial growth”, and the like as used herein refers to methods that result in any measurable decrease in microbial growth, where microbial growth includes any measurable decrease in the numbers and/or extent of microbial cells, spores, conidia, or mycelia. As used herein, “inhibiting growth of a plant pathogenic microbe” is also understood to include any measurable decrease in the adverse effects cause by microbial growth in a plant. Adverse effects of microbial growth in a plant include any type of plant tissue damage or necrosis, any type of plant yield reduction, any reduction in the value of the crop plant product, and/or production of undesirable microbial metabolites or microbial growth by-products including mycotoxins. As used herein, the phrase “inhibition of microbial growth” and the like, unless otherwise specified, can include inhibition in a plant, human or animal.

The phrases “percent identity” or “sequence identity” as used herein refer to the number of elements (i.e., amino acids or nucleotides) in a sequence that are identical within a defined length of two DNA, RNA segments in an alignment resulting in the maximal number of identical elements, and is calculated by dividing the number of identical elements by the total number of elements in the defined length of the aligned segments and multiplying by 100.

The phrase “transgenic” refers to an organism or progeny thereof wherein the organism's or progeny organism's DNA of the nuclear or organellar genome contains an inserted exogenous DNA molecule of 10 or more nucleotides in length. The phrase “transgenic plant” refers to a plant or progeny thereof wherein the plant's or progeny plant's DNA of the nuclear or plastid genome contains an introduced exogenous DNA molecule of 10 or more nucleotides in length. Such introduced exogenous DNA molecules can be naturally occurring, non-naturally occurring (e.g., synthetic and/or chimeric), from a heterologous source, or from an autologous source.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Further Description

Antimicrobial peptides and proteins referred to as defensin peptide variants are provided herein. The antimicrobial peptides and proteins can be applied directly to a plant, feedstuffs, or foodstuffs; applied to a plant in the form of microorganisms that produce the defensin peptide variant or protein, or the plants can be genetically edited to produce the defensin peptide variant or protein. The present disclosure also relates to recombinant or edited polynucleotides, microorganisms and plants transformed with the recombinant or edited polynucleotides, plants comprising genetically edited nuclear or plastid genomes encoding the defensin peptide variants and proteins and compositions comprising the defensin peptide variants and proteins useful in controlling pathogenic microbes including plant pathogenic microbes. In certain embodiments, the defensin variant protein comprising two defensin peptide variants or a defensin peptide variant and another peptide (including a defensin peptide variant or defensin peptide) can provide for improved inhibition of microbial growth when compared to a protein containing only one of the antimicrobial peptides found in the defensin variant protein. In certain embodiments, the defensin peptide variants and proteins provided herein are cation-tolerant. Such cation-tolerant defensins can be more effective than cation-sensitive defensins in providing effective control of plant pathogenic microbes in transgenic crops. Cation-tolerant defensins provided herein can function (e.g., inhibit plant pathogenic microbes including fungal pathogens) in the normal cation-rich physiological environment of plant tissues. Cation-tolerant defensins provided herein can also function (e.g., inhibit pathogenic microbes including fungal pathogens) in the normal cation-rich physiological environment of a subject (e.g., a human or animal) infected with pathogenic microbes.

Provided herein are recombinant polynucleotides comprising a polynucleotide encoding a first antifungal peptide operably linked to a polynucleotide comprising a promoter that is heterologous to the polynucleotide encoding the first antifungal protein. In certain embodiments, the first antifungal peptide is a defensin peptide variant.

Defensin peptide variants include peptides comprising, consisting essentially of, or consisting of the sequence: Xaa1-G-Xaa3-C-Xaa5-Xaa6-Xaa7-Xaa8-Xaa9-Xaa10-Xaa1 1-Xaa12-Xaa13-Xaa14-Xaa15-Xaa16-Xaa17-Xaa18-Xaa19-Xaa20-Xaa21-Xaa22-Xaa23, where Xaa1 is F, G, or absent; Xaa3 is A, G, Y, H, S, R or Dab; Xaa5 is H, L, S, N, R, K, or Dab; Xaa6, Xaa7, Xaa8, Xaa9, Xaa10, Xaa11, Xaa12, Xaa13, Xaa14, and Xaa15 are each independently R, K, H, N, D, F, G, Q, P, A, I, L, V, Y, S, or Dab, wherein one or more of Xaa8, Xaa9, Xaa10, Xaa11, Xaa12, Xaa13, Xaa14, and/or Xaa15 may be absent; Xaa16 is C, V, W, Y, or F; Xaa17 is L, I, W, Y, or F; Xaa18 is C, V, W, Y, or F; Xaa19 is K, T, F, W, or Y; Xaa20 is R, K, T, L, F, Q, or Dab, Xaa21 is N, H, K, R, P, Q, I, F, W, or Y; Xaa22 is N, H, K, P, Q, I, F, W, Y, or absent; and Xaa23 is C and is optionally amidated, wherein Dab is diaminobutyric acid (SEQ ID NO: 1). In certain embodiments, peptides comprising SEQ ID NO: 1 can further comprise one or more additional amino acids located C terminal to the C-terminus of SEQ ID NO:1, wherein said C-terminal amino acids are optionally amidated. In certain embodiments, peptides comprising SEQ ID NO: 1 can comprise a modified gamma core variant sequence GXCX3-8 (F/W/Y)(F/W/Y)(F/W/Y) (SEQ ID NO: 33), GXCX3-9(F/W/Y)(F/W/Y)(F/W/Y) (SEQ ID NO: 34). GXCX3-8 (F/W/Y) (SEQ ID NO: 43), or GXCX3-9(F/W/Y) (SEQ ID NO: 44). Defensin peptide variants also include peptides encompassed by the sequence: Xaa1-GRC-Xaa5-GFRRR(F/W/Y)(F/W/Y)(F/W/Y)XXXC—(NH₂), where Xaa1 is F, G, or absent; Xaa5 is R or K; Xaa14 is T, F, W, or Y; Xaa15 is R, K, or Dab; Xaa16 is I, F, W, or Y; and Xaa17 is C and is optionally amidated, wherein Dab is diaminobutyric acid (SEQ ID NO: 14). Defensin peptide variants further include peptides encompassed by the sequence Xaa1-G-Xaa2-C-Xaa5-Xaa6-Xaa7-GF-Xaa10-Xaa1l-Xaa12-(F/W/Y)(F/W/Y)(F/W/Y)-Xaa16-Xaa17-Xaa18-C—(NH₂), where Xaa1 is F, G, or absent, Xaa3 is A, F, W, Y, or absent; Xaa5 is H, R, or K; Xaa6 is R or K; Xaa7 is Q, R, or K; Xaa10 is G, R, or K; Xaa 11 is F, W, Y, R, or K; Xaa12 is A, F, W, Y, R, or K; Xaa16 is F, W, Y, R, or K; Xaa17 is R or K; Xaa18 is R or K; and the C-terminal cysteine is optionally amidated (SEQ ID NO: 17). Defensin variant peptides set forth herein do not comprise the corresponding full-length sequence of the wild-type defensin peptide of SEQ ID NO: 8, 16, 20, 23, 26, 29, 37, 38, 39, 40, or 41. Table 1 sets forth defensin peptide variants of SEQ ID NO: 1, 3-7 11, 13, and 14 as well as the GMA4C peptides of SEQ ID NO:2 and SEQ ID NO: 8. In certain embodiments, the aforementioned defensin peptide variants comprise, consist essentially of, or consist of: (i) 30 amino acid residues or less; or (ii) 15, 16, or 17 to 30 amino acid residues.

TABLE 1
Defensin peptides and Defensin peptide variants

| SEQ | | | Net | |
|---|---|---|---|---|
| ID NO: | Peptide | Sequence 3 | Charge | Hydrophobicity |
| 1 | Defensin Variant | X₁GX₃CX₅X₆X₇X₈X₉X₁₀X₁₁X₁₂X₁₃ | | |
| | Peptide | X₁₄X₁₅X₁₆X₁₇X₁₈X₁₉X₂₀X₂₁X₂₂ | | |
| | | C-(NH₂), wherein X₁ is F, G, or | | |
| | | absent; X₃ is A, G, Y, H, S, R or | | |
| | | Dab; X₅ is H, L, S, N, R, K, or | | |
| | | Dab; X₆, X₇, X₈, X₉, X₁₀, X₁₁, | | |
| | | X₁₂, X₁₃, X₁₄, and X₁₅ are each | | |
| | | independently R, K, H, N, D, F, | | |
| | | G, Q, P, A, I, L, V, Y, S, or Dab, | | |
| | | wherein one or more of X₈, X₉, | | |
| | | X₁₀, X₁₁, X₁₂, X₁₃, X₁₄, and/or | | |
| | | X₁₅ may be absent; X₁₆ is C, V, | | |
| | | W, Y, or F; X₁₇ is L, I, W, Y, or | | |
| | | F; X₁₈ is C, V, W, Y, or F; X₁₉ is | | |
| | | K, T, F, W, or Y; X₂₀ is R, K, T, | | |
| | | L, F, Q, or Dab, X₂₁ is N, H, K, | | |
| | | R, P, Q, I, F, W, or Y; X₂₂ is N, | | |
| | | H, K, P, Q, I, F, W, Y, or absent; | | |
| | | and C₂₃ and is optionally | | |
| | | amidated | | |
| 2 | GMA4C AC | GGRCRGFRRRCFCTTHC-NH₂ | 5.5 | 12% |
| 3 | GMA4C_V1A | GGRCKGFRRRCFCTRIC-NH₂ | 6 | 18% |
| 4 | GMA4C_V2A | GGRCRGFRRRCFCTRIC-NH₂ | 6 | 18% |
| 5 | GMA4C_V3A1 | GGRCRGFRRRVFVTRIC-NH₂ | 6 | 29% |
| 6 | GMA4C_V4A | FGRCRGFRRRCFCWRWC-NH₂ | 6 | 29% |
| 7 | GMA4C_V5A2 | FG(Dab)C(Dab)GF(Dab)(Dab) | 6 | 29% |
| | | (Dab)CFCW(Dab)WC-NH₂ | | |
| 8 | GMA4AC | GRCRGFRRRCFCTTHC | 5.5 | 12% |
| 9 | Gamma-core | GXCX3-9C | | |
| | consensus 1 | | | |
| 10 | Gamma-core | GXCX3-10C | | |
| | consensus 2 | | | |
| 11 | GMA4C_V4 | FGRCRGFRRRCFCWRWC | 6 | 29% |
| 12 | GMA4C_V5² | FG(Dab)C(Dab)GF(Dab)(Dab) | | |
| | | (Dab)CFCW(Dab)WC | | |
| 13 | GMA4C_V6A¹ | GGRCKGFRRRWFWTRIC-NH₂ | 6 | 29% |
| 14 | GMA4C_V7 | XGRCKGFRRR(F/W/Y)(F/W/Y) | | |
| | | (F/W/Y)XXXC-(NH₂) | | |
| 15 | GMA4C_V8A | GGRCKGFRRRWYWTRIC-NH₂ | 6 | 29% |
| 16 | GMA5A | GACHRQGFGFACFCYKKC | 3.5 | 33% |
| 17 | GMA5A_V1 | XGXCXXXGFXXXXFXXXXC-(NH₂) | | |
| 18 | GMA5A_V2 | GACHRQGFGFA(F/W/Y)(F/W/Y) | 3.5 | 44% |
| | | (F/W/Y)YKKC-(NH₂) | | |
| 19 | GMA5A_V3 | GACHRQGFGFAWFWYKKC-NH₂ | 3.5 | 44% |
| 20 | GMA5A_V4 | GACHRQGFGFAFFFYKKC-NH₂ | 3.5 | 44% |
| 21 | GMA6C | GGRCRGFRRRCFCTRPC | +6 | 12% |
| 22 | GMA6C_V1 | GGRCRGFRRR(F/W/Y)(F/W/Y) | +6 | 23.5% |
| | | (F/W/Y)TRPC-(NH₂) | | |
| 23 | GMA6C_V2 | GGRCRGFRRRWFWTRPC-NH₂ | +6 | 23.5% |
| 24 | GMAOe1C | GACLKNRHSKHYGCYCYYRHCY | 5.5 | 32% |
| 25 | GMAOe1C_V1 | GACLKNRHSKHYG(F/W/Y)(F/ | 5.5 | 41% |
| | | W/Y)(F/W/Y)YYRHCY-(NH₂) | | |
| 26 | GMAOe1C_V2 | GACLKNRHSKHYGWFWYYR | 5.5 | 41% |
| | | HCY-NH₂ | | |
| 27 | GMAOe7C | GGLCRGFRRRCFCTKHC | 5.5 | 18% |
| 28 | GMAOe7C_V1 | GGLCRGFRRR(F/W/Y)(F/W/Y) | 5.5 | 29% |
| | | (F/W/Y)TKHC-(NH₂) | | |
| 29 | GMAOe7C_V2 | GGLCRGFRRRWFWTKHC-NH₂ | 5.5 | 29% |
| 30 | GMASb1C | GGYCSSRRQICKCTLQC | 3 | 18% |
| 31 | GMASb1C_V1 | GGYCSSRRQI(F/W/Y)(F/W/Y) | 2 | 35% |
| | | (F/W/Y)TLQC-(NH₂) | | |
| 32 | GMASb1C_V2 | GGYCSSRRQIWFWTLQC-NH₂ | 2 | 35% |
| 33 | Modified | GXCX3-8 | | |
| | Gamma-core | (F/W/Y)(F/W/Y)(F/W/Y) | | |
| | consensus 1 | | | |
| 34 | Modified | GXCX3- | | |
| | Gamma-core | 9(F/W/Y)(F/W/Y)(F/W/Y) | | |
| | consensus 2 | | | |
| 35 | GMA1C | GRCRDDFRCWCTKNC | +2 | 13% |
| 36 | GMAIC_V1 | GRCRILFRCFCTKNC | +4 | 27% |
| 37 | GMANaDIC | GHCSKILRRCLCTKPC | +4.5 | 19% |
| 38 | GMARsAFP2C | GSCNYVFPAHKCICYFPC | +1.5 | 39% |
| 39 | GMADmAMP1C | GACHVRNGKHMCFCYFNC | +3 | 33% |
| 40 | GMAHsAFP1 | GACHYQFPSVKCFCTQNC | +1.5 | 28% |
| 41 | GMA5B | GACHRQGIGFACFCKKKC | +4.5 | 28% |
| 42 | GMAOe1_V3 | GACLKNRHSKHYGFFWYYRHCY | +5.5 | 36% |
| 43 | Modified | GXCX3-8(F/W/Y) | | |
| | Gamma-core | | | |
| | consensus 3 | | | |
| 44 | Modified | GXCX3-9(F/W/Y) | | |
| | Gamma-core | | | |
| | consensus 4 | | | |
| 45 | Modified | GXCX3-8(F/W/Y/L/V/I/M) | | |
| | Gamma-core | | | |
| | consensus 5 | | | |
| 46 | Modified | GXCX3-10(F/W/Y/L/V/I/M) | | |
| | Gamma-core | | | |
| | consensus 6 | | | |
| 47 | Modified | GXCX3-8(L/V/I/M) | | |
| | Gamma-core | (F/W/Y)(L/V/I/M) | | |
| | consensus 7 | | | |
| 48 | Modified | GXCX3-10(L/V/I/M) | | |
| | Gamma-core | (F/W/Y)(L/V/I/M) | | |
| | consensus 8 | | | |
| 49 | Modified | GXCX3-8(F/W/Y/L/V/I/M) | | |
| | Gamma-core | (F/W/Y)(F/W/Y/L/V/I/M) | | |
| | consensus 9 | | | |
| 50 | Modified | GXCX3-10(F/W/Y/L/V/I/M) | | |
| | Gamma-core | (F/W/Y)(F/W/Y/L/V/I/M) | | |
| | consensus 10 | | | |
| 51 | Modified | GXCX3- | | |
| | Gamma-core | 8(F/W/Y/L/V/I/M)(F/W/Y/L/V/I/M) | | |
| | consensus 11 | (F/W/Y/L/V/I/M) | | |
| 52 | Modified | GXCX3- | | |
| | Gamma-core | 10(F/W/Y/L/V/I/M)(F/W/Y/L/V/I/M) | | |
| | consensus 12 | (F/W/Y/L/V/I/M) | | |
| 587 | Modified | GXCX3- | | |
| | Gamma-core | 10(F/W/Y)(F/W/Y)(F/W/Y) | | |
| | consensus 13 | | | |
| 588 | Modified | GXCX3- | | |
| | Gamma-core | 12(F/W/Y)(F/W/Y)(F/W/Y) | | |
| | consensus 14 | | | |
| 589 | Modified | GXCX3- | | |
| | Gamma-core | 15(F/W/Y)(F/W/Y)(F/W/Y) | | |
| | consensus 15 | | | |
| 590 | Non-standard | GXCX3-12C | | |
| | Defensin gamma | | | |
| | core 1 | | | |
| 591 | Non-standard | GXCX3-15C | | |
| | Defensin gamma | | | |
| | core 2 | | | |

¹Substitution of cysteine at Xaa11 and Xaa13 with valine (GMA4C_V3A; SEQ ID NO: 5) or
with tryptophan (GMA4C_V6; SEQ ID NO: 13) can provide a cyclic peptide with a disulfide
bond between the residue 4 cysteine and residue 17 cysteine.
²Dab = diaminobutyric acid
³Amidation status: —NH2 is a C-terminal amidated peptide and —(NH2) is an optionally
amidated C-terminal peptide

In certain embodiments, defensin peptide variants of this disclosure are characterized as containing a defensin gamma-core peptide that is involved in the antifungal activity of plant defensins. A gamma-core peptide typically contains a net positive charge and has at least one hydrophobic amino acid. In certain embodiments, a defensin peptide variant can comprise the gamma-core consensus sequence of GXCX3-9C where X is any amino acid (SEQ ID NO: 9). In certain embodiments, a defensin peptide variant comprises a gamma-core peptide having a variant gamma-core consensus sequence of GXCX3-10C where X is any amino acid (SEQ ID NO: 10). In certain embodiments, a defensin peptide variant comprises the gamma-core consensus sequence of GXCX3-9C (SEQ ID NO: 9) or GXCX3-10C (SEQ ID NO:10); wherein X is preferentially selected from cationic and/or hydrophobic amino acids. In certain embodiments, a defensin peptide variant comprises SEQ ID NO: 1, comprises the gamma-core consensus sequence of GXCX3-9C (SEQ ID NO: 9) or GXCX3-10C (SEQ ID NO: 10); wherein X is preferentially selected from cationic and/or hydrophobic amino acids, wherein any of the variable (Xaa) amino acid residues of SEQ ID NO:1 comprise amino acid residues that maintains or increases the number of cationic and/or hydrophobic amino acids found in SEQ ID NO: 3-7. It is believed that the gamma-core peptide is involved in phospholipid- and/or sphingolipid-binding while specific amino acids outside the gamma-core motif are also involved in phospholipid- and sphingolipid-binding. With respect to SEQ ID NO: 1, the sequence between the first cysteine (C1) and the second cysteine (C2) from the amino terminus (or in certain embodiments a corresponding region in a defensin peptide variant) also contributes to antimicrobial activity.

Defensin peptide variants comprising variant defensin C-terminal peptides provided herein can also comprise substitutions of one or more of the conserved cysteine residues of a wild-type defensin C-terminal peptide sequence. Conserved cysteine residues which can be substituted can correspond to C1, C2, C3, or C4 in a defensin C-terminal peptide, C1 is the most N-terminal cysteine, C2 is the 2^{nd} most N-terminal cysteine, C3 is the 3^{rd} most N-terminal cysteine, and C4 is the most C-terminal cysteine; see FIG. 2). A non-limiting subset of reference defensin C-terminal peptides which comprise the C1, C2, C3, and C4 conserved cysteines include the GMA4C peptide of SEQ ID NO: 8, MtDef5A peptide of SEQ ID NO: 16, MtDef5A peptide of SEQ ID NO: 41, MtDef6 peptide of SEQ ID NO: 20, OeDef1 peptide of SEQ ID NO: 23, OeDef7 peptide of SEQ ID NO: 26, SbDef1 peptide of sequence ID NO: 29, an NaD1 C-terminal peptide (SEQ ID NO: 37), an RsAFP2 C-terminal peptide (SEQ ID NO: 38), a DmAMP1 C-terminal peptide (SEQ ID NO: 39), and an HsAFP1 (SEQ ID NO: 40) and can be substituted in at least the C2 and/or C3 positions to obtain a defensin peptide variant. Additional non-limiting reference defensin C-terminal peptides which comprise the C1, C2, C3, and C4 conserved cysteines include the defensin C-terminal peptides set forth in SEQ ID NO: 89 to 122. Additional non-limiting reference defensin C-terminal peptides which comprise the C1, C2, C3, and C4 conserved cysteines include defensin C-terminal peptides contained in the full length defensin peptides of SEQ ID NO: 123 to 434 and 436 to 576.

In certain embodiments, defensin peptide variants provided herein will lack a canonical gamma-core consensus sequence of GXCX3-9C (SEQ ID NO: 9) or GXCX3-10C (SEQ ID NO:10). In certain embodiments, defensin peptide variants provided herein will comprise a substitution of tryptophan, tyrosine, or phenylalanine for the C-terminal cysteine residue of the gamma core consensus sequence of SEQ ID NO: 9 or 10 (e.g., C2 in a reference SEQ ID NO: 8, 16, 20, 23, 26, 29, 37, 38, 39, 40, 41, or 89 to 122). In certain embodiments, defensin peptide variants provided herein comprise a substitution of both the C2 and C3 residues corresponding to the C2 and C3 residues in a reference C-terminal defensin peptide (e.g., SEQ ID NO: 8, 16, 20, 23, 26, 29, 37, 38, 39, 40, 41, or 89 to 122). A GMA4C variant having a valine substitution of C2 and C3 is set forth in SEQ ID NO: 5. A GMA4C variant having a tryptophan substitution of C2 and C3 is set forth in SEQ ID NO: 13. In certain embodiments, defensin peptide variants provided herein comprise a tryptophan, tyrosine or phenylalanine substitution of the C2 and C3 residues corresponding to the C2 and C3 residues in a reference C-terminal defensin peptide (e.g., SEQ ID NO: 8, 16, 20, 23, 26, 29, 37, 38, 39, 40, 41, or 89 to 122). In certain embodiments, defensin peptide variants set for the herein can further comprise a tryptophan, tyrosine, or phenylalanine residue in the amino acid position located between the C2 and C3 residues in a reference C-terminal defensin peptide (e.g., SEQ ID NO: 8, 16, 20, 23, 26, 29, 37, 38, 39, 40, 41, 89 to 122). Defensin peptide variants set forth herein comprising tryptophan, tyrosine or phenylalanine substitution of the C2 and C3 residues corresponding to the C2 and C3 residues in a reference C-terminal defensin peptide can comprise a modified gamma core variant sequence GXCX3-8 (F/W/Y)(F/W/Y)(F/W/Y) (SEQ ID NO: 33), GXCX3-9(F/W/Y)(F/W/Y)(F/W/Y) (SEQ ID NO: 34), GXCX3-10(F/W/Y)(F/W/Y)(F/W/Y) (SEQ ID NO: 587), GXCX3-12(F/W/Y)(F/W/Y)(F/W/Y) (SEQ ID NO: 588), GXCX3-15(F/W/Y)(F/W/Y)(F/W/Y) ((SEQ ID NO: 589), GXCX3-8 (F/W/Y) (SEQ ID NO: 43), or GXCX3-9(F/W/Y) (SEQ ID NO: 44). In certain embodiments, the aforementioned defensin peptide variants comprise, consist essentially of, or consist of: (i) 30 amino acid residues or less; or (ii) 15, 16, or 17 to 30 amino acid residues. In certain embodiments, the aforementioned defensin peptide variants comprise, consist essentially of, or consist of a peptide corresponding to the C-terminal end of a defensin peptide comprising the C1, C2, C3, and C4 conserved cysteines (e.g. SEQ ID NO: 8, 16, 20, 23, 26, 29, 37, 38, 39, 40, 41, 89-122 or contained in the full length defensin peptides of SEQ ID NO: 123 to 434 and 436 to 576) wherein a tryptophan, tyrosine or phenylalanine substitution of the C2 and C3 residues corresponding to the C2 and C3 residues in a reference C-terminal defensin peptide can result in a defensin variant peptide comprising a modified gamma core variant sequence GXCX3-8 (F/W/Y)(F/W/Y)(F/W/Y) (SEQ ID NO: 33), GXCX3-9(F/W/Y)(F/W/Y)(F/W/Y) (SEQ ID NO: 34), GXCX3-10(F/W/Y)(F/W/Y)(F/W/Y) (SEQ ID NO: 587), GXCX3-12(F/W/Y)(F/W/Y)(F/W/Y) (SEQ ID NO: 588), GXCX3-15(F/W/Y)(F/W/Y)(F/W/Y) ((SEQ ID NO: 589), GXCX3-8 (F/W/Y) (SEQ ID NO: 43), or GXCX3-9(F/W/Y) (SEQ ID NO: 44) and optionally comprise a peptide having just two (2) cysteine residues corresponding to the conserved C1 and C4 cysteines of a reference defensin C-terminal peptide.

In certain embodiments, additional defensin peptide variants lacking a canonical gamma-core consensus sequence of GXCX3-9C (SEQ ID NO: 9) or GXCX3-10C (SEQ ID NO:10) are provided. In certain embodiments, defensin peptide variants provided herein will comprise a substitution of tryptophan, tyrosine, phenylalanine, leucine, isoleucine, valine, or methionine for the C-terminal cysteine residue of the gamma core consensus sequence of SEQ ID NO: 9 or 10 (e.g., C2 in a reference SEQ ID NO: 8, 16, 20, 23, 26, 29, 37, 38, 39, 40, 41, or 89 to 122 or contained in the full length defensin peptides of SEQ ID NO: 123 to 434 and 436 to 576). In certain embodiments, defensin peptide variants provided herein comprise an independent substitution of both the C2 and C3 residues corresponding to the C2 and C3 residues in a reference C-terminal defensin peptide (e.g., SEQ ID NO: 8, 16, 20, 23, 26, 29, 37, 38, 39, 40, 41, or 89 to 122 or contained within the full length defensin peptides of SEQ ID NO: 123 to 434 and 436 to 576) with a tryptophan, tyrosine, phenylalanine, leucine, isoleucine, valine, or methionine residue. In certain embodiments, defensin peptide variants provided herein comprise substitutions of the C2 and C3 residues corresponding to the C2 and C3 residues in a reference C-terminal defensin peptide (e.g., SEQ ID NO: 8, 16, 20, 23, 26, 29, 37, 38, 39, 40, 41, or 89 to 122 or contained within the full length defensin peptides of SEQ ID NO: 123 to 434 and 436 to 576) with a tryptophan, tyrosine, phenylalanine, leucine, isoleucine, valine, or methionine residue. In certain embodiments, defensin peptide variants set for the herein can further comprise a tryptophan, tyrosine, phenylalanine, leucine, isoleucine, valine, or methionine residue in the amino acid position located between the C2 and C3 residues in a reference C-terminal defensin peptide (e.g., SEQ ID NO: 8, 16, 20, 23, 26, 29, 37, 38, 39, 40, 41, 89 to 122 or contained within the full length defensin peptides of SEQ ID NO: 123 to 434 and 436 to 576). Defensin peptide variants set forth herein comprising independent tryptophan, tyrosine, phenylalanine, leucine, isoleucine, valine, or methionione substitutions of the C2 and C3 residues corresponding to the C2 and C3 residues in a reference C-terminal defensin peptide can result in a defensin variant peptide comprising a modified gamma core variant sequence GXCX3-10(L/V/I/M)(F/W/Y)(L/V/I/M) (SEQ ID NO: 48), GXCX3-8(F/W/Y/L/V/I/M) (F/W/Y)(F/W/Y/L/V/I/M) (SEQ ID NO: 49), GXCX3-10(F/W/Y/L/V/I/M) (F/W/Y)(F/W/Y/L/V/I/M) (SEQ ID NO: 50), GXCX3-8(F/W/Y/L/V/I/M)(F/W/Y/L/V/I/M)(F/W/Y/L/V/I/M) (SEQ ID NO: 51), or GXCX3-10(F/W/Y/L/V/I/M)(F/W/Y/L/V/I/M)(F/W/Y/L/V/I/M) (SEQ ID NO: 52). In certain embodiments, the aforementioned defensin peptide variants comprise, consist essentially of, or consist of: (i) 30 amino acid residues or less; or (ii) 15, 16, or 17 to 30 amino acid residues. In certain embodiments, the aforementioned defensin peptide variants comprise, consist essentially of, or consist of a peptide corresponding to the C-terminal end of a defensin peptide comprising the C1, C2, C3, and C4 conserved cysteines (e.g. SEQ ID NO: 8, 16, 20, 23, 26, 29, 37, 38, 39, 40, 41, 89-122 or contained in the full length defensin peptides of SEQ ID NO: 123 to 434 and 436 to 576) wherein an independent tryptophan, tyrosine, phenylalanine, leucine, valine, isoleucine or methionine substitution of the C2 and C3 residues corresponding to the C2 and C3 residues in a reference C-terminal defensin peptide can result in a defensin variant peptide comprising a modified gamma core variant sequence of SEQ ID NO: 48, 49, 50, 51, or 52 and optionally comprise a peptide having just two (2) cysteine residues corresponding to the conserved C1 and C4 cysteines of a reference defensin C-terminal peptide.

In certain embodiments, a defensin peptide variant comprises an amino acid sequence having at least 70%, 75%, 80%, 82%, 85%, 90%, 92%, 94%, 95%, or 100% sequence identity across the entire length of SEQ ID NO: 3-7, 12-15, 17-20, 22, 23, 25, 26, 28, 29, 31, 32, 42, 578, 579, 581, 582, 584, or 585 wherein said peptide does not comprise the corresponding full-length or C-terminal sequence of the defensin peptide of SEQ ID NO: 8, 16, 20, 23, 26, 29, 37, 38, 39, 40, 41, 123 to 434, or 436 to 576. In certain embodiments, such defensin peptide variants will having at least 70%, 75%, 80%, 82%, 85%, 90%, 92%, 94%, 95%, or 100% sequence identity across the entire length of SEQ ID NO: 3-7, 12-15, 17-20, 22, 23, 25, 26, 28, 29, 31, 32, 42, 578, 579, 581, 582, 584, or 585 further comprise the gamma-core consensus sequence of GXCX3-9C (SEQ ID NO: 9) or GXCX3-10C (SEQ ID NO: 10); wherein X is any amino acid or wherein X is selected from cationic and/or hydrophobic amino acids. In certain embodiments, such defensin peptide variants will having at least 70%, 75%, 80%, 82%, 85%, 90%, 92%, 94%, 95%, or 100% sequence identity across the entire length of SEQ ID NO: 3-7, 12-15, 17-20, 22, 23, 25, 26, 28, 29, 31, 32, 42, 578, 579, 581, 582, 584, or 585 further comprise a modified gamma core variant sequence GXCX3-8 (F/W/Y)(F/W/Y)(F/W/Y) (SEQ ID NO: 33), GXCX3-9(F/W/Y)(F/W/Y)(F/W/Y) (SEQ ID NO: 34), GXCX3-10(F/W/Y)(F/W/Y)(F/W/Y) (SEQ ID NO: 587), GXCX3-12(F/W/Y)(F/W/Y)(F/W/Y) (SEQ ID NO: 588), GXCX3-15(F/W/Y)(F/W/Y)(F/W/Y) ((SEQ ID NO: 589), GXCX3-8 (F/W/Y) (SEQ ID NO: 43), GXCX3-9(F/W/Y) (SEQ ID NO: 44), GXCX3-8(F/W/Y/L/V/I/M) (SEQ ID NO: 45), GXCX3-10(F/W/Y/L/V/I/M) (SEQ ID NO: 46), GXCX3-8(L/V/I/M)(F/W/Y)(L/V/I/M) (SEQ ID NO: 47), GXCX3-10(L/V/I/M)(F/W/Y)(L/V/I/M) (SEQ ID NO: 48), GXCX3-8(F/W/Y/L/V/I/M) (F/W/Y)(F/W/Y/L/V/I/M) (SEQ ID NO: 49), GXCX3-10(F/W/Y/L/V/I/M) (F/W/Y)(F/W/Y/L/V/I/M) (SEQ ID NO: 50), GXCX3-8(F/W/Y/L/V/I/M)(F/W/Y/L/V/I/M)(F/W/Y/L/V/I/M) (SEQ ID NO: 51), or GXCX3-10(F/W/Y/L/V/I/M)(F/W/Y/L/V/I/M)(F/W/Y/L/V/I/M) (SEQ ID NO: 52). In certain embodiments, such defensin peptide variants will have at least 70%, 75%, 80%, 82%, 85%, 90%, 92%, 94%, 95%, or 100% sequence identity across the entire length of SEQ ID NO: 3-7, 2-15, 17-20, 22, 23, 25, 26, 28, 29, 31, 32, 42, 578, 579, 581, 582, 584, or 585 have anet positive charge of at least about 5.8 at neutral pH and/or a hydrophobicity percentage of at least about 15%. In certain embodiments, a first structural feature of the defensin peptide variants is a net positive charge at neutral pH. In certain embodiments, the defensin peptide variants will have a net positive charge at neutral pH of at least +2, +3, +3.5, +4, +5, +6, +7, +8, +9, or +10. In certain embodiments, the defensin peptide variants will have a net positive charge at neutral pH of at least +3, +3.5, +4, +5, +6, or +7 to about +8, +9, or +10. In certain embodiments, the hydrophobicity percentage of such defensin peptide variants is at least about 15% to 30%, about 16% to 19%, or about 28% to 30%. In certain embodiments, the aforementioned defensin peptide variants comprise, consist essentially of, or consist of: (i) 30 amino acid residues or less; or (ii) 15, 16, or 17 to 30 amino acid residues. In certain embodiments, any of the aforementioned defensin peptide variants comprise a peptide having just two (2) cysteine residues and optionally comprise the two cysteine residues corresponding to the conserved C1 and C4 cysteines of a reference defensin C-terminal peptide.

In certain embodiments, defensin peptide variants comprise amino acid substitutions which increase or maintain the net positive charge of the peptide at neutral pH and/or increase or maintain the hydrophobicity of the peptide. Amino acid substitutions in SEQ ID NO: 3-7, 12-15, 17-20, 22, 23, 25, 26, 28, 29, 31, 32, 42, 578, 579, 581, 582, 584, or 585 which can maintain net positive charge of the peptide at neutral pH include substitution of a lysine, arginine, or Dab (diaminobutyric acid) residue in SEQ ID NO: 3-7, 12-15, 17-20, 22, 23, 25, 26, 28, 29, 31, 32, 42, 578, 579, 581, 582, 584, or 585 with a different amino acid residue selected from the group consisting of lysine, arginine, Dab (diaminobutyric acid), or other non-naturally occurring amino acid which is positively charged at neutral pH. Amino acid substitutions in SEQ ID NO: 3-7, 12-15, 17-20, 22, 23, 25, 26, 28, 29, 31, 32, 42, 578, 579, 581, 582, 584, or 585 which can increase net positive charge at neutral pH include substitution of a polar (e.g. cysteine or threonine) or non-polar (e.g., glycine) residue in SEQ ID NO: 3-7, 12, 12-15, 17-20, 22, 23, 25, 26, 28, 29, 31, 32, 42, 578, 579, 581, 582, 584, or 585 with a different amino acid residue selected from the group consisting of lysine, arginine, Dab (diaminobutyric acid), or other non-naturally occurring amino acid residue which is positively charged at neutral pH. Amino acid substitutions in SEQ ID NO: 3-7, 12-15, 17-20, 22, 23, 25, 26, 28, 29, 31, 32, 42, 578, 579, 581, 582, 584, or 585 which can maintain hydrophobicity of the peptide include substitution of a glycine, valine, phenylalanine, or isoleucine residue in SEQ ID NO: 3-7, 12-15, 17-20, 22, 23, 25, 26, 28, 29, 31, 32, 42, 578, 579, 581, 582, 584, or 585 with a different amino acid residue selected from the group consisting of glycine, alanine, valine, leucine, phenylalanine, isoleucine, or methionine. Amino acid substitutions in SEQ ID NO: 3-7, 12-15, 17-20, 22, 23, 25, 26, 28, 29, 31, 32, 42, 578, 579, 581, 582, 584, or 585 which can increase hydrophobicity of the peptide include substitution of a polar (e.g. cysteine or threonine) residue in SEQ ID NO: 3-7, 12-15, 17-20, 22, 23, 25, 26, 28, 29, 31, 32, 42, 578, 579, 581, 582, 584, or 585 with a different amino acid residue selected from the group consisting of glycine, alanine, valine, leucine, phenylalanine, or isoleucine. In certain embodiments, such substitutions which increase or maintain net positive charge or hydrophobicity of the peptide will comprise a defensin peptide variant having the gamma-core consensus sequence of GXCX3-9C (SEQ ID NO: 9) or GXCX3-10C (SEQ ID NO:10). In certain embodiments, such substitutions which increase or maintain net positive charge or hydrophobicity of the peptide will comprise a defensin peptide variant having the modified gamma core variant sequence GXCX3-8 (F/W/Y)(F/W/Y)(F/W/Y) (SEQ ID NO: 33) or GXCX3-9(F/W/Y)(F/W/Y)(F/W/Y) (SEQ ID NO: 34), GXCX3-10(F/W/Y)(F/W/Y)(F/W/Y) (SEQ ID NO: 587), GXCX3-12(F/W/Y)(F/W/Y)(F/W/Y) (SEQ ID NO: 588), GXCX3-15(F/W/Y)(F/W/Y)(F/W/Y) ((SEQ ID NO: 589) GXCX3-8 (F/W/Y)(F/W/Y)(F/W/Y) (SEQ ID NO: 33), GXCX3-8 (F/W/Y) (SEQ ID NO: 43), or GXCX3-9(F/W/Y) (SEQ ID NO: 44). In certain embodiments, such substitutions which increase or maintain net positive charge or hydrophobicity of the peptide will comprise a defensin peptide variant having the modified gamma core variant sequence can comprise a modified gamma core variant sequence of SEQ ID NO: 45, SEQ ID NO: 46, SEQ ID NO: 47, SEQ ID NO: 48, SEQ ID NO: 49, SEQ ID NO: 50, SEQ ID NO: 51, or SEQ ID NO: 52. In certain embodiments, the aforementioned defensin peptide variants comprise, consist essentially of, or consist of: (i) 30 amino acid residues or less; or (ii) 15, 16, or 17 to 22, 24, 16, 28 or 30 amino acid residues. In certain embodiments, any of the aforementioned defensin peptide variants comprise a peptide having just two (2) cysteine residues and optionally comprise the two cysteine residues corresponding to the conserved C1 and C4 cysteines of a reference defensin C-terminal peptide.

In certain embodiments, one or more amino acids in any of the aforementioned or other variant defensin peptide variant sequences are substituted with another amino acid(s), the charge and polarity of which is similar to that of the original amino acid, i.e., a conservative amino acid substitution. Substitutes for an amino acid within the defensin peptide variant or protein, or defensin peptide sequence can be selected from other members of the class to which the originally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include: (1) acidic (anionic, negatively charged) amino acids such as aspartic acid and glutamic acid; (2) basic (cationic, positively charged) amino acids such as arginine, histidine, and lysine; (3) neutral polar amino acids such as glycine, serine, threonine, cysteine, cystine, tyrosine, asparagine, and glutamine; (4) neutral nonpolar (hydrophobic) amino acids such as alanine, leucine, isoleucine, valine, proline, phenylalanine, tryptophan, and methionine. Conservative amino acid changes within defensin peptide sequences can be made by substituting one amino acid within one of these groups with another amino acid within the same group. Biologically functional equivalents of defensin peptide variants can have 10 or fewer conservative amino acid changes, seven or fewer conservative amino acid changes, or five, four, three, two, or one conservative amino acid changes. The encoding nucleotide sequence (e.g., gene, plasmid DNA, cDNA, or synthetic DNA) will thus have corresponding base substitutions, permitting it to encode biologically functional equivalent forms of the defensin peptide variants. Certain semi-conservative substitutions in defensin peptide variants including: (i) the substitution of a neutral polar amino acid residue with a neutral nonpolar (hydrophobic) amino acid residue; or (ii) the substitution of a neutral nonpolar (hydrophobic) amino acid residue with a neutral polar amino acid residue are also provided. In particular, semi-conservative substitutions of a neutral polar tyrosine residue with a hydrophobic amino acid residue are provided. Biologically functional equivalents of defensin peptide variants can have 10 or fewer semi conservative amino acid changes, seven or fewer semi-conservative amino acid changes, or five, four, three, two, or one semi-conservative amino acid changes.

Nucleic acid molecules encoding any of the aforementioned defensin peptide variants are also provided herein. Recombinant DNA molecules comprising the aforementioned nucleic acid molecules are also provided herein and in particular recombinant DNA molecules comprising a heterologous promoter that is operably linked to the aforementioned nucleic acid molecules are also provided herein.

A defensin peptide variant provided herein can be operably linked to another defensin peptide variant, defensin, or antimicrobial peptide via a spacer peptide sequence that is not susceptible to cleavage by an endoproteinase, including a plant endoproteinase. Such peptide linker sequences that join peptides in multimeric or multi-domain proteins have been disclosed (Argos, 1990; George R A, Heringa (2002)). Examples of suitable peptide sequences from multimeric or multi-domain proteins that can be used as spacer domains include immunoglobulin hinge regions from immunoglobulins, a linker between the lipoyl and E3 binding domain in pyruvate dehydrogenase (Turner et ah, 1993), a linker between the central and C-terminal domains in cysteine proteinase (P9; Mottram et ah, 1989), and functional variants thereof. Spacer peptides for use in the defensin variant proteins can also be wholly or partially synthetic peptide sequences. Such synthetic spacer peptides are designed to provide for a flexible linkage between the at least one defensin peptide variant and another peptide (including a defensin peptide variant or defensin peptide) and to be resistant to cleavage by endogenous plant or other endoproteinases. In certain embodiments, the length of the synthetic spacer peptide can be between about 3, 4, 8, 10, 12, or 16 and about 20, 24, 28, 30, 40, or 50 amino acid residues in length. In certain embodiments, the synthetic spacer peptide can comprise a glycine-rich or glycine/serine containing peptide sequence. The composition and design of peptides suitable for flexible linkage of protein domains described in the literature (Chen et al., 2013) can be adapted for use as spacer peptides in the defensin variant proteins provided herein. Spacer peptides useful for joining defensin monomers described in US Patent Appln. Publications US20190194268 and US20190185877, which are each incorporated herein by reference in their entireties, can also be used to join defensin peptide variants disclosed herein to other defensin peptide variants, defensins, antimicrobial peptides, or other peptides.

A defensin peptide variant provided herein can be operably linked to another defensin peptide variant, defensin, or antimicrobial peptide via a linker peptide sequence that is susceptible to cleavage by an endoproteinase, including a plant endoproteinase. In certain embodiments, the resultant defensin variant protein can be expressed in a cell such that the endoproteinase cleaves the defensin variant protein to provide at least one defensin peptide variant and another peptide (including a defensin peptide variant or defensin peptide). Such defensin variant proteins can be provided in a cellular compartment (e.g., cytoplasm, mitochondria, plastid, vacuole, or endoplasmic reticulum) or extracellular space (i.e., to the apoplast) having an endoproteinase that cleaves the linker peptide. Cleavable linker peptides are disclosed in WO2014078900, Vasivarama and Kirti, 2013a, Frangois et al, Vasivarama and Kirti, 2013b, and WO2017127558 can be used in the defensin variant proteins provided herein.

Expression cassettes that provide for expression of the defensin peptide variant in monocotyledonous plants, dicotyledonous plants, or both can be constructed. Such defensin peptide variant expression cassette construction can be effected either in a plant expression vector or in the genome of a plant. Expression cassettes are DNA constructs wherein various promoter, coding (e.g. defensin peptide variant encoding), and polyadenylation sequences are operably linked. In general, expression cassettes typically comprise a promoter that is operably linked to a sequence of interest, which is operably linked to a polyadenylation or terminator region. In certain instances including the expression of recombinant or edited polynucleotides in monocot plants, it can also be useful to include an intron sequence. When an intron sequence is included it is typically placed in the 5′ untranslated leader region of the recombinant or edited polynucleotide. In certain instances, it can also be useful to incorporate specific 5′ untranslated sequences in a recombinant or edited polynucleotide to enhance transcript stability or to promote efficient translation of the transcript. Expression cassettes and vectors for expression of other defensin peptides or proteins in plants, including those disclosed in U.S. patent Ser. No. 10/253,328 which is incorporated herein by reference in its entirety, can be adapted for expression of the defensin peptide variants in transgenic plants. Any of the defensin peptide variant expression vectors can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. The aforementioned methods of introducing transgenes are described in US Patent Appl. Pub. No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton), each of which are incorporated herein by reference in their entirety.

In certain embodiments, a plant comprising a recombinant or edited polynucleotide encoding a defensin peptide variant can be obtained by using techniques that provide for site specific insertion of heterologous DNA into the genome of a plant (e.g., by CRISPR, TALEN, or Zinc finger nuclease-mediated gene editing). In certain embodiments, a DNA fragment encoding at least a defensin peptide variant is site specifically integrated into the genome to a plant cell, tissue, part, or whole plant to create a sequence within that genome that encodes a defensin peptide variant. Examples of methods for inserting foreign DNA at specific sites in the plant genome with site-specific nucleases such as meganucleases or zinc-finger nucleases are at least disclosed in Voytas, 2013. Examples of methods for inserting foreign DNA into the plant genome with clustered regularly interspaced short palindromic repeats (CRISPR)-associated (Cas)-guide RNA technology and a Cas endonuclease are at least disclosed by Svitashev et al., 2015; Murovec et al., 2017; Kumar and Jain, 2015; and in US Patent Appl. Pub. No. 20150082478, which is specifically incorporated herein by reference in its entirety.

Expression of defensin peptide variants in yeast is also specifically contemplated herein. The construction of expression vectors for production of heterologous proteins in various yeast genera is well established. In general, such expression vectors typically comprise a promoter that is operably linked to a sequence of interest which is operably linked to a polyadenylation or terminator region. Examples of yeast genera that have been used to successfully express heterologous genes include Candida, Kluveromyces, Hansuela, Pichia, Saccharomyces, Schizosaccharomyces, and Yarrowia. A general description of expression vectors and transformation systems for Saccharomyces is found in Kingsman et al (1985). Expression vectors and transformation systems useful for yeasts other than Saccharomyces are described in Reiser et al (1990). Expression cassettes and vectors for expression of other defensin peptides or proteins in yeast, including those disclosed in U.S. patent Ser. No. 10/253,328, which is incorporated herein by reference in its entirety, can be adapted for expression of the defensin peptide variants in yeast.

Expression of defensin peptide variants in bacteria is also specifically contemplated herein. The construction of expression vectors for production of heterologous proteins in various bacterial systems have been described. In general, such expression vectors typically comprise a promoter that is operably linked to a sequence encoding peptide sequence(s) of interest (e.g., a signal transit peptide region at the n-terminus of a defensin variant peptide) which is operably linked to a procaryotic terminator region. Examples of bacterial genera that have been used to successfully express heterologous genes include Acinetobacter, Alcaligenes, Azotobacter, Bacillus, Escherichia, Lactobacillus, Lactococcus, Streptomyces, and Pseudomonas. E. coli expression systems useful for production of proteins comprising disulfide bonds and that can be adapted for use in expression of the defensin variant peptides provided herein include those described in are described in Kuddus et al. (2017) Biotechnol Prog 233:1520-1528. doi: 10.1002/btpr.Protein Science 2508, Kiedzierska et al. (2008) Protein Expr Purif 60, 82-88, Chang et al. (2015) Amino Acids 47, 579-587, Buchko et al. (2018) Protein Science 27, 1611-1623, Marques et al. (2008) J Appl Microbiol 106, 1640-1648, and Pazgier et al. (2006) Protein Expr Pur 49, 1-8. Systems for expressing proteins which comprise disulfide bonds can be adapted for expression of the Defensin peptides in E. coli include those disclosed in U.S. Patent Publication No. US 2020/0172915, which is incorporated herein by reference in its entirety, and in Berkmen, M. Protein Expr Purif. 2012; 82(1):240-51. doi: 10.1016/j.pep.2011.10.009.

Other bacterial expression systems for expression of the useful for production of proteins comprising disulfide bonds and that can be adapted for use in expression of the defensin variant peptides provided herein include those described in U.S. Pat. No. 10,604,761, which is incorporated herein by reference in its entirety.

Also provided are antimicrobial compositions for agricultural, pharmaceutical, or veterinary use comprising either an antimicrobial plant, or antimicrobial human or veterinary, pathogenic microbe inhibitory amount (“antimicrobial effective amount”) of one or more the present isolated, purified antimicrobial defensin peptide variants, or biologically functional equivalents thereof. Such compositions can comprise one, or any combination of, defensin peptide variants or proteins disclosed herein, and an agriculturally, pharmaceutically, or veterinary-practicably acceptable carrier, diluent, or excipient. As indicated below, other components relevant in agricultural and therapeutic contexts can be included in such compositions as well. The antimicrobial compositions can be used for inhibiting the growth of, or killing, defensin peptide variant-susceptible pathogenic microbes associated with plant, human or animal microbial infections. Such antimicrobial compositions can be formulated for topical administration, and applied topically to either plants, the plant environment (including soil), or humans or animals. Such antimicrobial compositions can be formulated for enteral, parenteral, and/or intravenous administration of the composition, and administered to a subject in need thereof; such subject can be a human, livestock, poultry, fish, or a companion animal. The defensin peptide variants can be formulated alone, in any combination with one another, and either of these can additionally be formulated in combination with other conventional antimicrobial therapeutic compounds such as, by way of non-limiting example, polyene antimicrobials; imidazole, triazole, and thiazole antimicrobials; allylamines; and echinocandins that are routinely used in human and veterinary medicine. Administration of the compositions that comprise defensin peptide variants to a human or animal subject in need thereof can be accomplished via a variety of routes that include topical application, enteral administration, parenteral administration, and/or intravenous administration. The antimicrobial peptides and compositions can be used to control microbial pathogens or contaminants including: (i) a bacterial pathogen of plants or animals, wherein the bacterial pathogen is optionally a member of the group Enterobacteriaceae and optionally wherein the bacterial pathogen is a Salmonella sp., Escherichia sp., or Listeria sp.; (ii) is a Fusarium sp., Alternaria sp., Aphenomyces sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Leptosphaeria sp., Gaeumannomyces sp., Puccinia sp. Septoria sp., Penicillium sp., Lasiodiplodia sp., Phomopsis sp., Mycosphaerella sp., Golovinomyces sp., Erisyphe sp., Albugo sp., Setosphaeria sp., Cochiobolus sp., Helminthosporium sp., Diplodia sp., or Stenocarpella sp.; (iii) an Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, Coccidiomyces or Blastomyces species; (iv) a Candida species and wherein the Candida species is Candida albicans (C. albicans), C. auris, C. glabrata, C parapsilosis, C. tropicalis, or C. krusei; or (v) dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

Agricultural compositions comprising any of the present defensin peptide variant molecules alone, or in any combination, can be formulated as described in, for example, Winnacker-Kuchler (1986) Chemical Technology, Fourth Edition, Volume 7, Hanser Verlag, Munich; van Falkenberg (1972-1973) Pesticide Formulations, Second Edition, Marcel Dekker, N.Y.; and K. Martens (1979) Spray Drying Handbook, Third Edition, G. Goodwin, Ltd., London. Formulation aids, such as carriers, inert materials, surfactants, solvents, and other additives are also well known in the art, and are described, for example, in Watkins, Handbook of Insecticide Dust Diluents and Carriers, Second Edition, Darland Books, Caldwell, N.J., and Winnacker-Kuchler (1986) Chemical Technology, Fourth Edition, Volume 7, Hanser Verlag, Munich. Using these formulations, it is also possible to prepare mixtures of the present defensin peptide variants and proteins with other pesticidally active substances, fertilizers, and/or growth regulators, etc., in the form of finished formulations or tank mixes.

Whether alone or in combination with other active agents, the present antimicrobial defensin peptide variants can be applied to subjects or plants at a concentration in the range of from about 0.1 pg/ml to about 100 mg/ml, or from about 5 μg/ml to about 5 mg/ml, at a pH in the range of from about 3.0 to about 9.0. Such compositions can be buffered using, for example, phosphate buffers between about 1 mM and 1 M, about 10 mM to about 100 mM, or about 15 mM to about 50 mM. In the case of low buffer concentrations, a salt can be added to increase the ionic strength. In certain embodiments, a sodium salt, including NaCl, in the range of from about 1 mM to about 1 M, about 1 mM, 5 mM, or 10 mM to about 20 mM, 50 mM, 100 mM, 150 mM, or 200 mM, or about 10 mM to about 100 mM, can be added or provided in compositions comprising defensin peptide variants and proteins. In certain embodiments, a potassium salt, including KCl, in the range of about 1 mM, 5 mM, or 10 mM to about 20 mM, 50 mM, 100 mM, 150 mM, or 200 mM can be added or provided in compositions comprising defensin peptide variants and proteins. In certain embodiments, a calcium salt, including CaCl₂), in the range of about 0.1 mM, 0.5 mM, or 1 mM to about 2 mM, 5 mM, 10 mM, or 20 mM can be added or provided in compositions comprising defensin peptide variants.

Numerous conventional microbial antibiotics and chemical antimicrobial agents (e.g., fungicides) with which the present defensin peptide variants and proteins can be combined are described in Worthington and Walker (1983) The Pesticide Manual, Seventh Edition, British Crop Protection Council. These include, for example, polyoxins, nikkomycins, carboxy amides, aromatic carbohydrates, carboxines, morpholines, inhibitors of sterol biosynthesis, and organophosphorous compounds. In addition, azoles, triazoles and echinocandins fungicides can also be used. Other active ingredients which can be formulated in combination with the present antimicrobial peptides and proteins include, for example, insecticides, attractants, sterilizing agents, acaricides, nematicides, and herbicides. U.S. Pat. No. 5,421,839, which is incorporated herein by reference in its entirety, contains a comprehensive summary of the many active agents with which substances such as the present antimicrobial defensin peptide variants and proteins can be formulated.

Agriculturally useful antimicrobial compositions encompassed herein also include those in the form of host cells, such as bacterial and microbial cells, capable of producing the defensin peptide variants and proteins, and which can colonize plants, including roots, shoots, leaves, or other parts of plants. The term “plant-colonizing microorganism” is used herein to refer to a microorganism that is capable of colonizing the any part of the plant itself and/or the plant environment, including, and which can express the present defensin variant antimicrobial peptides and proteins in the plant and/or the plant environment. A plant colonizing micro-organism is one that can exist in symbiotic or non-detrimental relationship with a plant in the plant environment. U.S. Pat. No. 5,229,112, which is incorporated herein by reference in its entirety, discloses a variety of plant-colonizing microorganisms that can be engineered to express antimicrobial peptides and proteins, and methods of use thereof, applicable to the defensin variant antimicrobial peptides and proteins disclosed herein. Plant-colonizing microorganisms expressing the presently disclosed defensin variant antimicrobial peptides and proteins useful in inhibiting microbial growth in plants include bacteria selected from the group consisting of Bacillus spp. including Bacillus thuringiensis, Bacillus israelensis, and Bacillus subtilis, Candidatus Liberibacter asiaticus; Pseudomonas spp.; Arthrobacter spp., Azospyrillum spp., Clavibacter spp., Escherichia spp.; Agrobacterium spp., for example A. radiobacter, Rhizobium spp., Erwinia spp. Azotobacter spp., Azospirillum spp., Klebsiella spp., Alcaligenes spp., Rhizobacterium spp., Xanthomonas spp., Ralstonia spp. and Flavobacterium spp. In certain embodiments, the microorganism is a yeast selected from the group consisting of Saccharomyces cerevisiae, Pichia pastoris, and Pichia methanolica. In certain embodiments, the plant colonizing microorganism can be an endophytic bacteria or microbe. When applying the present defensin peptide variant molecules to the rhizosphere, rhizosphere-colonizing bacteria from the genus Pseudomonas are particularly useful, especially the fluorescent pseudomonads, e.g., Pseudomonas fluorescens, which is especially competitive in the plant rhizosphere and in colonizing the surface of the plant roots in large numbers. Examples of suitable phylloplane (leaf) colonizing bacteria are P. putida, P. syringae, and Erwinia species.

### EMBODIMENTS

The following numbered embodiments form part of the disclosure:

1a. A peptide comprising the sequence SEQ ID NO: 1, wherein said peptide does not comprise the corresponding full-length sequence of the defensin peptide of SEQ ID NO: 8, 16, 20, 23, 26, 29, 37, 38, 39, 40, or 41; optionally wherein said peptide comprises a modified gamma-core consensus sequence GXCX3-8 (F/W/Y)(F/W/Y)(F/W/Y) set forth in SEQ ID NO: 33, GXCX3-9(F/W/Y)(F/W/Y)(F/W/Y) set forth in SEQ ID NO: 34, GXCX3-8 (F/W/Y) (SEQ ID NO: 43), or GXCX3-9(F/W/Y) (SEQ ID NO: 44); and/or optionally wherein the C-terminal cysteine residue or C-terminal amino acid residue is amidated.

1b. A peptide having at least 50%, 55%, 60%, 68%, 75%, 82%, or 94% sequence identity across the entire length of any one of SEQ ID NO: 3, 4, 5, or 6, wherein the peptide is not identical to SEQ ID NO: 8, optionally wherein any amino acid substitution in said sequence increases or maintains the net positive charge at neutral pH and/or increases or maintains hydrophobicity of the peptide, and optionally wherein the C-terminal cysteine residue is amidated.

1c. A peptide having at least 50%, 55%, 60%, 68%, 75%, 82%, 94%, 95%, or 100% sequence identity across the entire length of SEQ ID NO:7, 12, 13, 14, 15, 17-20, 22, 23, 25, 26, 28, 29, 31, 32, 42, 578, 579, 581, 582, 584, or 585, wherein the peptide is not identical to SEQ ID NO: 8, 16, 20, 23, 26, 29, 37, 38, 39, 40, or 41, optionally wherein the peptide of SEQ ID NO:5, SEQ ID NO: 13, 14, 15, 18, 19, 20, 22, 23, 26, 28, 29, 31, 32, 42, 42, 578, 579, 581, 582, 584, or 585 comprises a disulfide bond between the two cysteine residues and optionally wherein any substitution increases or maintains the net positive charge at neutral pH and/or increases or maintains hydrophobicity of the peptide, and optionally wherein the C-terminal cysteine residue or C-terminal amino acid residue is amidated.

1d. A defensin C-terminal peptide variant comprising conserved C1 and C4 cysteine residues corresponding to N-terminal and C-terminal cysteines of a reference defensin C-terminal peptide, wherein conserved C2 and C3 cysteine residues of the reference defensin C-terminal peptide are independently substituted with tryptophan, tyrosine, phenylalanine, leucine, valine, isoleucine, or methionine; and optionally wherein the defensin peptide variant has a net positive charge of at least 3, 3.5, 4, 5, or 6 and a hydrophobic amino acid content at least 18%.

1e. A C-terminal defensin peptide variant comprising a modified gamma-core consensus sequence GXCX3-8 (F/W/Y)(F/W/Y)(F/W/Y) (SEQ ID NO: 33), GXCX3-9(F/W/Y)(F/W/Y)(F/W/Y) (SEQ ID NO: 34), GXCX3-10(F/W/Y)(F/W/Y)(F/W/Y) (SEQ ID NO: 587), GXCX3-12(F/W/Y)(F/W/Y)(F/W/Y) (SEQ ID NO: 588), GXCX3-15(F/W/Y)(F/W/Y)(F/W/Y) ((SEQ ID NO: 589), GXCX3-8 (F/W/Y) (SEQ ID NO: 43), GXCX3-9(F/W/Y) (SEQ ID NO: 44), GXCX3-8(F/W/Y/L/V/I/M) (SEQ ID NO: 45), GXCX3-10(F/W/Y/L/V/I/M) (SEQ ID NO: 46), GXCX3-8(L/V/I/M)(F/W/Y)(L/V/I/M) (SEQ ID NO: 47), GXCX3-10(L/V/I/M)(F/W/Y)(L/V/I/M) (SEQ ID NO: 48), GXCX3-8(F/W/Y/L/V/I/M) (F/W/Y)(F/W/Y/L/V/I/M) (SEQ ID NO: 49), GXCX3-10(F/W/Y/L/V/I/M) (F/W/Y)(F/W/Y/L/V/I/M) (SEQ ID NO: 50), GXCX3-8(F/W/Y/L/V/I/M)(F/W/Y/L/V/I/M)(F/W/Y/L/V/I/M) (SEQ ID NO: 51), or GXCX3-10(F/W/Y/L/V/I/M)(F/W/Y/L/V/I/M)(F/W/Y/L/V/I/M) (SEQ ID NO: 52), optionally wherein the peptide further comprises a conserved C4 cysteine residue, optionally wherein the cysteine residue in the modified gamma-core consensus sequence forms a disulfide bond with the conserved C4 cysteine residue, and/or optionally wherein the defensin peptide variant has a net positive charge of at least 3, 3.5, 4, 5, or 6 and a hydrophobic amino acid content at least 18%.

1f. A peptide comprising a modified gamma-core consensus sequence GXCX3-8 (F/W/Y)(F/W/Y)(F/W/Y) (SEQ ID NO: 33), GXCX3-9(F/W/Y)(F/W/Y)(F/W/Y) (SEQ ID NO: 34), GXCX3-10(F/W/Y)(F/W/Y)(F/W/Y) (SEQ ID NO: 587), GXCX3-12(F/W/Y)(F/W/Y)(F/W/Y) (SEQ ID NO: 588), GXCX3-15(F/W/Y)(F/W/Y)(F/W/Y) ((SEQ ID NO: 589), GXCX3-8 (F/W/Y) (SEQ ID NO: 43), or GXCX3-9(F/W/Y) (SEQ ID NO: 44), GXCX3-8(F/W/Y/L/V/I/M) (SEQ ID NO: 45), GXCX3-10(F/W/Y/L/V/I/M) (SEQ ID NO: 46), GXCX3-8(L/V/I/M)(F/W/Y)(L/V/I/M) (SEQ ID NO: 47), GXCX3-10(L/V/I/M)(F/W/Y)(L/V/I/M) (SEQ ID NO: 48), GXCX3-8(F/W/Y/L/V/I/M) (F/W/Y)(F/W/Y/L/V/I/M) (SEQ ID NO: 49), GXCX3-10(F/W/Y/L/V/I/M) (F/W/Y)(F/W/Y/L/V/I/M) (SEQ ID NO: 50), GXCX3-8(F/W/Y/L/V/I/M)(F/W/Y/L/V/I/M)(F/W/Y/L/V/I/M) (SEQ ID NO: 51), or GXCX3-10(F/W/Y/L/V/I/M)(F/W/Y/L/V/I/M)(F/W/Y/L/V/I/M) (SEQ ID NO: 52), wherein the peptide further comprises a second C-terminal cysteine residue located C-terminal to the cysteine residue in the modified gamma-core consensus sequence, and wherein the peptide has a net positive charge of at least 3, 3.5, 4, 5, or 6 and a hydrophobic amino acid content at least 18%, optionally wherein the peptide comprises a disulfide bond between the two cysteine residues.

2. The peptide of embodiment 1a, 1b, 1c, 1d, 1e, or 1f, wherein said peptide comprises; (i) an amino acid sequence selected from the group consisting of SEQ ID NO:3, SEQ ID NO:4, SEQ ID NO:5, SEQ ID NO:6, SEQ ID NO:7, SEQ ID NO: 12, SEQ ID NO: 13, SEQ ID NO: 14, SEQ ID NO: 15, 17-20, 22, 23, 25, 26, 28, 29, 31, 32, 42, 578, 579, 581, 582, 584, or 585; optionally wherein the peptide of SEQ ID NO:5, SEQ ID NO: 13, SEQ ID NO: 14, 15, 18, 19, 20, 22, 23, 26, 28, 29, 32, 42, 578, 579, 581, 582, 584, or 585 comprises a disulfide bond between the two cysteine residues; (ii) an amino acid sequence having at least 50%, 55%, 60%, 68%, 75%, 82%, 94%, 95%, or 100% sequence identity across the entire length of SEQ ID NO:7, SEQ ID NO: 12, SEQ ID NO: 13, SEQ ID NO: 14, 15, 17-20, 22, 23, 25, 26, 28, 29, 31, 32, 18, 19, 20, 22, 23, 26, 28, 29, 32, 42, 578, 579, 581, 582, 584, or 585, wherein said peptide does not comprise the corresponding full-length sequence of the defensin peptide of SEQ ID NO: 8, 16, 20, 23, 26, 29, 37, 38, 39, 40, or 41; and optionally wherein the peptide comprises a disulfide bond between the two cysteine residues; or (iii) wherein said peptide exhibits antimicrobial activity.

3. The peptide of embodiment 1a, 1b, 1c, 1d, 1e, 1f, or 2, wherein the peptide comprises, consists essentially of, or consists of: (i) 30 amino acid residues or less; (ii) 15, 16, or 17 to 30 amino acid residues; or (iii) 15, 16, or 17 to 30 amino acid residues and contains two cysteine residues.

4. A composition comprising the peptide of any of one embodiments 1a, 1b, 1c, 1d, 1e, or 1f to 3 and an agriculturally, pharmaceutically, or veterinary-practicably acceptable carrier, diluent, or excipient.

5. The composition of embodiment 4, wherein the peptide is provided at a concentration of about 0.1, 0.5, 1.0, or 5 μg/ml to about 1, 5, 20, 50, or 100 mg/ml or at a concentration of about 0.1, 0.5, 1.0, or 5 pg/gram to about 1, 5, 20, 50, or 100 mg/gram and optionally wherein the composition comprises a sodium salt at a concentration of at least 100 mM and/or a calcium salt at a concentration of at least 2 mM.

6. A method for: (i) preventing or reducing crop damage by a plant pathogenic microbe; or (ii) preventing contamination of plants, plant parts, seeds, feedstuff obtained therefrom, or foodstuff obtained therefrom with an undesirable microbe, comprising the step of contacting a plant, a plant seed, other part of said plant, feedstuff obtained therefrom, or foodstuff obtained therefrom with an effective amount of the composition of embodiment 4 or embodiment 5.

7. The method of embodiment 6, wherein the plant pathogenic microbe or undesirable microbe is: (i) a bacterial pathogen of plants or animals, wherein the bacterial pathogen is optionally a member of the group Enterobacteriaceae and optionally wherein the bacterial pathogen is a Salmonella sp., Escherichia sp., or Listeria sp.; or (ii) is a Fusarium sp., Alternaria sp., Aphenomyces sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Leptosphaeria sp., Gaeumannomyces sp., Puccinia sp. Septoria sp., Penicillium sp., Lasiodiplodia sp., Phomopsis sp., Mycosphaerella sp., Golovinomyces sp., Erisyphe sp., Albugo sp., Setosphaeria sp., Cochliobolus sp., Helminthosporium sp., Diplodia sp., or Stenocarpella sp.

8. A medical device comprising the device and the composition of embodiment 4 or 5, wherein the device comprises at least one surface that is topically coated and/or impregnated with the composition.

9 The medical device of embodiment 8, wherein said device is a stent, a catheter, a contact lens, a condom, a patch, or a diaphragm.

10. A method for treating, preventing, or inhibiting a microbial infection in a subject in need thereof comprising administering to said subject an effective amount of the composition of embodiment 4 or 5.

11. The method of embodiment 10, wherein said administration comprises topical, enteral, parenteral, and/or intravenous introduction of the composition.

12. The method of embodiment 10 or 11, wherein the subject is a human, livestock, poultry, fish, or a companion animal.

13. The method of embodiment 12, wherein the microbial infection is of a mucosal membrane, eye, skin, and/or a nail and the composition is applied to the mucosal membrane, eye, skin, and/or nail.

14. The method of any one of embodiments 10 to 13, wherein the microbial infection is by a dermatophyte, and wherein the dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

15. The method of any one of embodiments 10 to 13, wherein the microbial infection is by: (i) a bacterial pathogen of animals, wherein the bacterial pathogen is optionally a member of the group Enterobacteriaceae and optionally wherein the bacterial pathogen is a Salmonella sp., Escherichia sp., or Listeria sp.; or (ii) an Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, Coccidiomyces or Blastomyces species.

16. The method of any one of embodiments 10 to 13, wherein the microbial infection is by a Candida species and wherein the Candida species is Candida albicans (C. albicans), C. auris, C. glabrata, C parapsilosis, C. tropicalis, or C. krusei.

17. The composition of embodiment 4 or 5 for use in a method of treating, preventing, or inhibiting microbial infection in a subject in need thereof.

18. The composition of embodiment 17, wherein the subject is a human, livestock, poultry, fish, or a companion animal.

19. A plant part that is at least partly coated with the composition of embodiment 4 or 5.

20. The plant part of embodiment 19, wherein the part is a seed and the seed is optionally a corn, soybean, wheat, rice, cotton, Brassica sp., or tomato seed.

21. The plant part of embodiment 19, wherein the plant part is a fruit, vegetable, or flower.

22. A recombinant polynucleotide comprising a polynucleotide encoding an antimicrobial peptide of embodiment 1a, 1b, 1c, 1d, 1e, 1f, 2, or 3, wherein the polynucleotide encoding the first antimicrobial peptide is operably linked to a polynucleotide comprising a promoter which is heterologous to the polynucleotide encoding the first antimicrobial peptide, optionally wherein any amino acid substitution in said sequence increases or maintains the net positive charge and/or increases or maintains hydrophobicity of the peptide.

23. The recombinant polynucleotide of embodiment 22, wherein the recombinant polynucleotide further comprises a polynucleotide encoding: (i) a transit peptide, a vacuolar targeting peptide, and/or an endoplasmic reticulum targeting peptide; (ii) a plastid targeting peptide; and/or (iii) a polyadenylation or transcriptional termination signal, wherein the polynucleotides of (i), (ii), and/or (iii) are operably linked to the polynucleotide encoding the antimicrobial peptide.

24. The recombinant polynucleotide of embodiment 22 or 23, wherein the polynucleotide encoding the first antimicrobial peptide is inserted into a heterologous nuclear or plastid genome of a cell and operably linked to an endogenous promoter located in the heterologous nuclear or plastid genome.

25. A plant nuclear or plastid genome comprising a polynucleotide encoding an antimicrobial peptide of embodiment 1a, 1b, 1c, 1d, 1e, 1f, 2, or 3, and wherein the polynucleotide is heterologous to the nuclear or plastid genome and wherein the polynucleotide is operably linked to an endogenous promoter of the nuclear or plastid genome.

26. A cell comprising the recombinant polynucleotide of embodiment 22 or the genome of embodiment 25, wherein the cell is optionally a bacterial, yeast, or plant cell.

27. A plant comprising the recombinant polynucleotide of embodiment 22 or the genome of embodiment 25.

28. A plant part of the plant of embodiment 26, wherein the plant part comprises the recombinant polynucleotide or genome, optionally wherein the plant part is a seed, stem, leaf, root, tuber, flower, or fruit.

29. A method for producing plant seed that provides plants resistant to infection by a plant pathogenic microbe that comprises the steps of: (i) selfing or crossing the plant of embodiment 22; and (ii) harvesting seed that comprises the recombinant polynucleotide of the plant from the self or cross, thereby producing plant seed that provide plants resistant to infection by a plant pathogenic microbe.

### PUBLICATIONS CITED

Argos, (1990) J Mol Biol. February 20; 211(4):943-58.
Broadley, M. R., Bowen, H. C., Cotterill, H. L., Hammond, J. P., Meacham, M. C., Mead, A., and White, P. J. (2003). Journal of Experimental Botany 54, 1431-1446
Buchko et al. (2018) Protein Science 27, 1611-1623.
Chang et al. (2015) Amino Acids 47, 579-587.
Chen et al., (2013) Adv Drug Deliv Rev.; 65(10): 1357-1369.
da Silva Conceicao, A., and Broekaert, W. F. (1999). Plant Defensins. In Pathogenesis-related proteins in plants, S. Muthukrishnan, ed (New York: CRC Press), pp. 247-260.
Franqois et al., Plant Physiology (2002) 128: 1346-1358.
George R A, and Heringa (2002) J Protein Eng. 15(11):871-879.
Hanks, J. N., et al., (2005). Plant Mol Biol 58, 385-399.
Kerenga B K, et al., (2019) Front. Microbiol. 10:795. doi: 10.3389/fmicb.2019.00795
Kiedzierska, et al. (2008) Protein Expr Purif 60, 82-8.
Kingsman S M, et al., (1985) Biotechnol Genet Eng Rev. 3:377-416.
Kuddus et al. (2017). Biotechnol Prog 233:1520-1528. doi: 10.1002/btpr.Protein Science 2508.
Kumar, V. and Jain, M. (2015) J Exp Bot 66: 47-57.
Lacerda et ah, Frontiers in Microbio. (2014) 5(116): 1-10.
Lay, F. T., and Anderson, M. A. (2005) Curr Protein Pept Sci 6, 85-101.
Marques et al. (2008) J Appl Microbiol 106, 1640-1648.
Mottram et al., FEBS Lett. (1989) 258(2):211-215.
Murovec et al., Plant Biotechnol J. 2017 August; 15(8): 917-926.
Pazgier et al. (2006) Protein Expr Pur 49, 1-8.
Reiser J, et al., (1990) Adv Biochem Eng Biotechnol.; 43:75-102.
Sagaram et al., (2011) PLOS ONE 6: e18550.
Sagaram et al., (2013) PLoS ONE, 8(12): e82485.
Shabala, S., and Pottosin, I. I. (2010) Signal. Commun. Plants 87-110.
Svitashev et al., (2015) Plant Physiology, 169 (2): 931-945.
Spelbrink, R. G., et al., (2004). Plant Physiol 135, 2055-2067.
Terras, F. R., et al., (1992). J Biol Chem 267, 15301-15309.
Thomma, B. P. H. J., Cammue, B. P. A., and Thevissen, K. (2002). Planta 216, 193-202.
Turner et al., (1993) Protein Eng. 6(1): 101-108.
Voytas, D. Annual Review of Plant Biology, Vol. 64: 327-350, 2013.
Vasivarama and Kirti (2013a) Plant Cell Tiss Organ Cult, 115:309-319.
Vasivarama and Kirti (2013b) Funct Integr Genomics 13:435-443.
Vriens, K., et al. Molecules 2014, 19, 12280-12303; doi: 10.3390/molecules190812280.
White, P., and Karley, A. (2010). Potassium. In Cell Biology of Metals and Nutrients; Hell, R., Mendel, R. R., Eds.; Springer: Berlin/Heidelberg, Germany., 199-224

### EXAMPLES

### Example 1

Crude peptides (GMA4C-AC, GMA4C_V1, GMA4C_V2 and GMA4C_V3) were synthesized chemically by Biomatik Inc, Canada, and GMA4C_V4 and GMA4C_V5 by Alan Scientific Inc. (USA) and were further purified, using a linear gradient of acetonitrile/water mixture, in a C-18 reverse phase HPLC (Agilent Technologies, USA). HPLC fractions were lyophilized and resuspended in nuclease-free water. Concentration of each peptide was determined using the BCA assay performed according to the manufacturer's protocol (Thermo-Fisher Scientific, USA). Antifungal activity was determined in assays in SFM culture media. The SFM culture media comprises K₂HPO₄ (2.5 mM), MgSO₄ (50 μM), CaCl₂) (50 μM), FeSO₄ (5 μM), CoCl₂ (0.1 μM), CuSO₄ ((0.1 μM), Na₂MoO₄ (2 μM), H₃BO₃ (0.5 μM), KI (0.1 μM), ZnSO₄ (0.5 μM), MnSO₄ (0.1 μM), glucose (10 g/liter), asparagine (1 g/liter), methionine (20 mg/liter), myo-inositol (2 mg/liter), biotin (0.2 mg/liter), thiamine-HCL (1 mg/liter), and pyridoxine-HCL (0.2 mg/liter), pH 7.0.)

TABLE 2
Antifungal Activity of Peptides

| | SEQ ID | B. cinerea | F. graminearum | F. oxysporum | P. capsici |
|---|---|---|---|---|---|
| Peptide | NO. | MIC (μM) | MIC (μM) | MIC (μM) | MIC (μM) |
| GMA4C_AC ¹ | 2 | 1.5-3.0 | 3 | 6 | 1.5 |
| GMA4C_V1A | 3 | 1.5 | 3 | 6 | 3 |
| GMA4C_V2A | 4 | 3 | 3-6 | — | — |
| GMA4C_V3A | 5 | 1.5 | 3 | — | — |
| GMA4C_V4 | 11 | 1.5 | 3 | — | — |
| Non-amidated | | | | | |
| GMA4C_V5 | 12 | 1.5 | 3 | — | — |
| Non-amidated | | | | | |
| GMA4C_V6A | 13 | 0.75 | 1.5 | — | — |

¹ C-terminus is amidated.

TABLE 3
Comparison of GMA4C wild-type and variant peptides

| | | MIC Values | MIC Values | | |
|---|---|---|---|---|---|
| Peptide | SEQ ID | (μM) against | (μM) against | Salt | |
| Name | NO: | B. cinerea | F. graminearum | tolerance | Comments |
| GMA4C_AC | 2 | 3 | 6 | + | Chemical synthesis, |
| | | | | | amidated |
| GMA4C_V1A | 3 | 1.5 | 3 | ++ | Chemical synthesis, |
| | | | | | amidated |
| GMA4C_V2A | 4 | 3 | 6 | ++ | Chemical synthesis, |
| | | | | | amidated |
| GMA4C_V3A | 4 | 3 | 3 | ++ | Chemical synthesis, |
| | | | | | amidated |
| GMA4C_V4A | 6 | 1.5 | 1.5 | +++ | Chemical synthesis, |
| | | | | | amidated |
| GMA4C_V5A | 7 | 3 | 3 | + | Chemical synthesis, |
| | | | | | amidated |
| GMA4C_V6A | 13 | 0.75 | 1.5 | +++ | Chemical synthesis, |
| | | | | | amidated |

Antifungal activity in vitro in low cationic conditions: The GMA4C variants GMA4C_VIA, GMA4C_V3A, GMA4C_V4A, and GMA4C_V5A exhibit antifungal activity equivalent to the wild-type GMA4C_AC control in vitro against B. cinerea, F. graminearum, F. oxysporum and P. capsici. The GMA4C variant GMA4C_V2A exhibit a two-fold reduction in antifungal activity relative to the wild-type GMA4C_AC control in vitro against B. cinerea and F. graminearum. GMA4C_GMA4C_V6A exhibits a four-fold increase in antifungal activity relative to the wild-type GMA4C_AC control in vitro against B. cinerea.

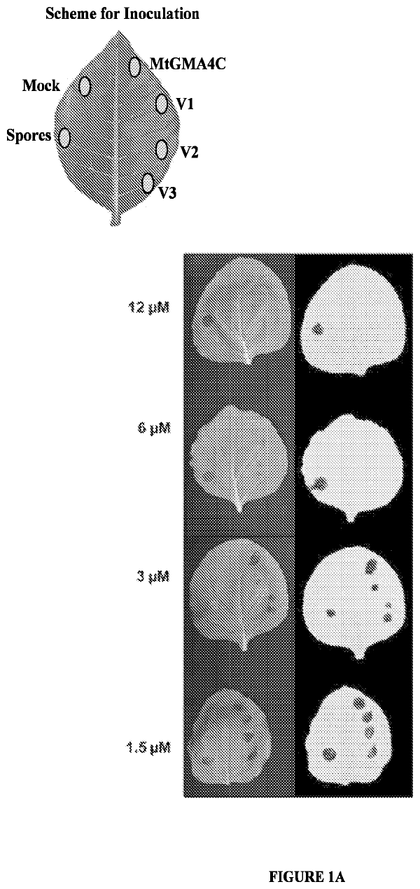

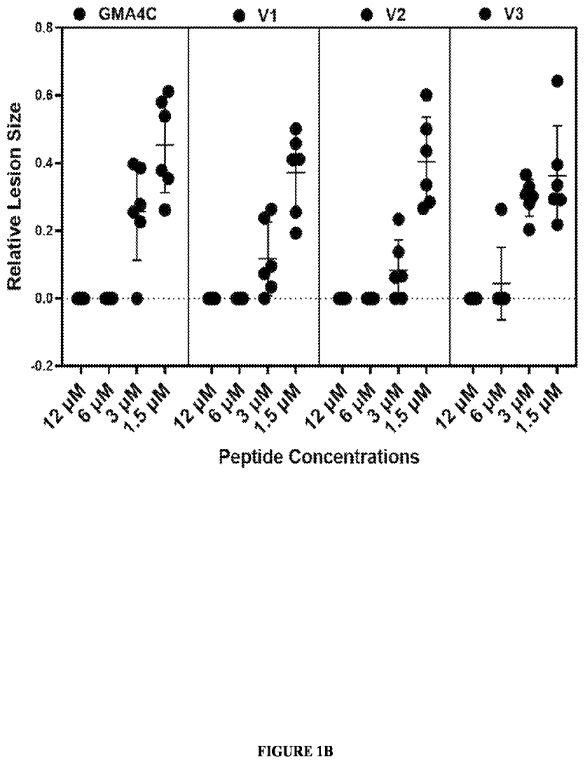

Antifungal activity in planta. When applied on detached Nicotiana benthamiana leaves, GMA4C_V1A and GMA4C_V3A are more potent than GMA4C_AC or GMA4C_V2A in reducing gray mold disease symptoms caused by B. cinerea as shown in FIGS. 1A and 1B. When applied on tomato leaves, GMA4C_V1A is more effective in reducing disease symptoms caused by P. capsici than GMA4C_AC.

Antifungal activity in vitro in presence of cations. When tested for antifungal activity against B. cinerea in the presence of 100 mM NaCl, 100 mM KCl, or 2 mM CaCl₂), all of the peptides including GMA4C_AC retain their antifungal activity in presence of 100 mM NaCl or 100 mM KCl. However, GMA4C_V1A and GMA4C_V4A have 2-fold more potent activity than GMA4C_AC. In presence of 2 mM CaCl₂), only GMA4C_V1A and GMA4C_V4A inhibit fungal growth at 6 μM, whereas other peptides such as plant defensins MtDef4 or OeDef1 show little or no activity at this concentration.

TABLE 4
GMA4C and its variants retain antifungal activity against B. cinerea in presence of salts.

| | | | SFM + 100 mM | SFM + 100 mM | SFM + 2 mM |
|---|---|---|---|---|---|
| | | SFM MIC | NaCl MIC | KCl MIC | CaCl₂ MIC |
| Peptide | SEQ ID NO: | (μM) ¹ | (μM) ¹ | (μM) ¹ | (μM) ¹ |
| GMA4C_AC ¹ | 2 | 3 | 3 | 6 | >6 |
| | | | | | (a few |
| | | | | | germinated |
| | | | | | at 6) |
| GMA4C_V1A | 3 | 3 | 1.5 | 3 | 6 |
| GMA4C_V2A | 4 | 6 | 3 | 6 | >6 |
| GMA4C_V3A | 5 | 3 | 1.5-3 | >6 | >6 |
| GMA4C_V4A | 6 | 1.5-3 | 1.5 | 1.5 | 3-6 |
| GMA4C_V4 | 11 | 3 | 3 | 3 | 6 |
| Non-amidated | | | | | |
| GMA4C_V5A | 7 | 6 | 6 | >6 | >6 |
| GMA4C_V5 | 12 | 6 | 6 | >6 | >6 |
| Non-amidated | | | | | (very |
| | | | | | sensitive) |
| GMA4C_V6A | 13 | 1.5 | 0.75-1.5 | 1.5-3 | 3-6 |

¹ The Minimal Inhibitory Concentration (MIC) is the concentration of the peptide at which there is no significant growth of the microorganism relative to the growth of the microorganism in growth medium lacking the compound, protein, or peptide.

Antifungal activity against human fungal pathogens: GMA4C_V1A and GMA4C_V3A show antifungal activity against C. auris, and C. glabrata in the RPMI medium rich in cations, but the parental MtDef4 defensin does not show activity.

TABLE 5
Composition of Roswell Park Memorial Institute
1640 culture medium (RPMI media; with glutamine
and phenol red but without bicarbonate)

| Constituent | Water, g/L | Constituent | Water, g/L |
|---|---|---|---|
| L-arginine (free base) | 0.2 | Biotin | 0.0002 |
| L-asparagine (anhydrous) | 0.05 | D-pantothenic acid | 0.00025 |
| L-aspartic acid | 0.02 | Choline chloride | 0.003 |
| L-cystine * 2HC1 | 0.0652 | Folic acid | 0.001 |
| L-glutamic acid | 0.02 | Myo-inositol | 0.035 |
| L-glutamine | 0.3 | Niacinamide | 0.001 |
| Glycine | 0.01 | PABA | 0.001 |
| L-histidine (free base) | 0.015 | Pyridoxine HCI | 0.001 |
| L-hydroxyproline | 0.02 | Riboflavin | 0.0002 |
| L-isoleucine | 0.05 | Thiamine HCI | 0.001 |
| L-leucine | 0.05 | Vitamin B₁₂ | 0.000005 |
| L-lysine * HCI | 0.04 | Calcium Nitrate H₂O | 0.01 |
| L-methionine | 0.015 | Potassium chloride | 0.4 |
| L-phenylalanine | 0.015 | Magnesium sulfate | 0.04884 |
| | | (anhydrous) | |
| L-proline | 0.02 | Sodium chloride | 6 |
| L-serine | 0.03 | Sodium phosphate, | 0.8 |
| | | dibasic (anhydrous) | |
| L-threonine | 0.02 | D-glucose | 2 |
| L-tryptophan | 0.005 | Glutathione, reduced | 0.001 |
| L-tyrosine * 2Na | 0.02883 | Phenol red, Na | 0.0053 |
| L-valine | 0.02 | | |

TABLE 6
GMA4C_V1A and GMA4C_V3A exhibit antifungal activity in vitro
in the RPMI medium against Candida auris and C. glabrata.

| | Candida | Candida | Candida |
|---|---|---|---|
| | albicans | auris | glabrata |
| Peptide | MIC (μg/ml) ¹ | MIC (μg/ml) ¹ | MIC (μg/ml) ¹ |
| MtDef4 | >46.9 | 46.9 | >46.9 |
| GMA4C_V1A | 46.9 | 11.9 | 11.9 |
| GMA4C_V3A | >46.9 | 23.9 | 23.9 |

¹ The Minimal Inhibitory Concentration (MIC) is the concentration of the protein or peptide at which there is no significant growth of the microorganism relative to the growth of the microorganism in growth medium lacking the compound, protein, or peptide.

### Example 2. Activity of GMA4C-Variants Against Pathogenic Microbes

Testing for antifungal activity used half-strength potato dextrose broth for peptides and RPMI for comparator antifungals fluconazole and voriconazole. CLSI M27 and M38 methodologies used for to measure MICs. Minimal Inhibitory Concentration (MIC) is the concentration of the compound, protein, or peptide at which there is no significant growth of the microorganism relative to the growth of the microorganism in growth medium lacking the compound, protein, or peptide.

All testing was performed in RPMI buffered with 0.165M MOPS. The concentration range for peptides in potato dextrose broth was 0.06-2 mcg/ml, the concentration range for fluconazole was 0.125-64 mcg/ml, and the concentration range for voriconazole was 0.03-16 mcg/ml. MICs were determined at 24-72 hours.

TABLE 7
Formula for Potato Dextrose Broth
Potato Dextrose Broth

| Value | | Ingredients and conditions |
|---|---|---|
| 1000 | ml | water |
| 4 g (from 200 g infused potato) | | Potatoes (sliced, unpeeled, washed) |
| 20 | g | Glucose |
| pH | 5.6 | Final pH |
| 25 | C. | Temperature |

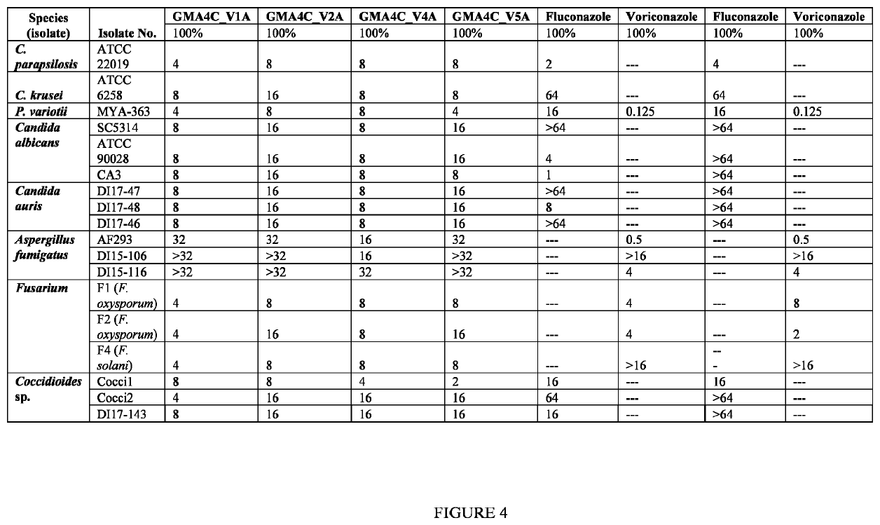

TABLE 8
MIC values for GMA4C variants. The complete table with data
for fluconazole and Voriconazole are provided in FIG. 4.

| Species | | GMA4C_V1A | GMA4C_V2A | GMA4C_V4A | GMA4C_V5A |
|---|---|---|---|---|---|
| (isolate) | Isolate No. | 100% | 100% | 100% | 100% |
| C. | ATCC | 4 | 8 | 8 | 8 |
| parapsilosis | 22019 | | | | |
| C. krusei | ATCC | 8 | 16 | 8 | 8 |
| | 6258 | | | | |
| P. variotii | MYA-363 | 4 | 8 | 8 | 4 |
| Candida | SC5314 | 8 | 16 | 8 | 16 |
| albicans | ATCC | 8 | 16 | 8 | 16 |
| | 90028 | | | | |
| | CA3 | 8 | 16 | 8 | 8 |
| Candida | DI17-47 | 8 | 16 | 8 | 16 |
| auris | DI17-48 | 8 | 16 | 8 | 16 |
| | DI17-46 | 8 | 16 | 8 | 16 |
| Aspergillus | AF293 | 32 | 32 | 16 | 32 |
| fumigatus | DI15-106 | >32 | >32 | 16 | >32 |
| | DI15-116 | >32 | >32 | 32 | >32 |
| Fusarium | F1 (F. | 4 | 8 | 8 | 8 |
| | oxysporum) | | | | |
| | F2 (F. | 4 | 16 | 8 | 16 |
| | oxysporum) | | | | |
| | F4 (F. | 4 | 8 | 8 | 8 |
| | solani) | | | | |
| Coccidioides | Cocci1 | 8 | 8 | 4 | 2 |
| sp. | Cocci2 | 4 | 16 | 16 | 16 |
| | DI17-143 | 8 | 16 | 16 | 16 |

TABLE 9
Antifungal activity of GMA4C wild-type and its variants against
plant fungal pathogens in SFM.

| | | | MIC (μM) | MIC (μM) |
|---|---|---|---|---|
| SEQ | | | Botrytis | Fusarium |
| ID NO: | Peptide | Sequence | cinera | graminearum |
| 2 | GMA4C_AC | GGRCRGFRRRCFCTTHC-NH₂ | 3 | 3 |
| 3 | GMA4C_V1A | GGRCKGFRRRCFCTRIC-NH₂ | 3 | 3 |
| 4 | GMA4C_V2A | GGRCRGFRRRCFCTRIC-NH₂ | 6 | 3-6 |
| 5 | GMA4C_V3A¹ | GGRCRGFRRRVFVTRIC-NH₂ | 3 | 3 |
| 6 | GMA4C_V4A | FGRCRGFRRRCFCWRWC-NH₂ | 1.5 | — |
| 7 | GMA4C_V5A² | FG(Dab)C(Dab)GF(Dab)(Dab) | 6 | — |
| | | (Dab)CFCW(Dab)WC-NH₂ | | |
| 8 | GMA4AC | GRCRGFRRRCFCTTHC | 6 | 6 |
| 11 | GMA4C_V4 | FGRCRGFRRRCFCWRWC | 3 | 3 |
| 12 | GMA4C_V5² | FG(Dab)C(Dab)GF(Dab)(Dab) | 6 | 3 |
| | | (Dab)CFCW(Dab)WC | | |
| 13 | GMA4C_V6¹ | GGRCKGFRRRWFWTRIC-NH₂ | 0.75 | 1.5 |
| 14 | GMA4C_V7 | XGRCKGFRRR(F/W/Y)(F/W/Y) | — | |
| | | (F/W/Y)XXXC-(NH₂) | | |
| 15 | GMA4C_V8 | GGRCKGFRRRWYWTRIC-NH₂ | — | — |

TABLE 10
Antifungal activity of GMA4C wild-type and its variants
against plant fungal pathogens in SFM + salts

| | | SFM + 100 mM | SFM + 100 mM | SFM + 2 mM |
|---|---|---|---|---|
| Peptide | SFM MIC | NaCl MIC | KCl MIC | CaCl₂ MIC |
| (SEQ ID NO) | (μM) | (μM) | (μM) | (μM) |
| GMA4C_AC | 3 | 3 | 6 | >6 (a few |
| (SEQ ID NO: 2) | | | | germinated at 6) |
| GMA4C_V1A | 3 | 1.5 | 3 | 6 |
| (SEQ ID NO: 3) | | | | |
| GMA4C_V2A | 6 | 3 | 6 | >6 |
| (SEQ ID NO: 4) | | | | |
| GMA4C_V3A | 3 | 1.5-3 | >6 | >6 |
| (SEQ ID NO: 5) | | | | |
| GMA4C_V4A | 1.5-3 | 1.5 | 1.5 | 3-6 |
| (SEQ ID NO: 6) | | | | |
| GMA4C_V4 | 3 | 3 | 3 | 6 |
| Non-amidated | | | | |
| (SEQ ID NO: 11) | | | | |
| GMA4C_V5A | 6 | 6 | >6 | >6 |
| (SEQ ID NO: 7) | | | | |
| GMA4C_V5 | 6 | 6 | >6 | >6 (very |
| Non-amidated | | | | sensitive) |
| (SEQ ID NO: 12) | | | | |
| GMA4C_V6A | 1.5 | 0.75-1.5 | 1.5-3 | 3-6 |
| (Seq ID NO:13) | | | | |

TABLE 11
Antifungal activity of peptides against human yeast
pathogens in half-strength potato dextrose broth

| | Candida albicans | Candida auris | Candida glabrata |
|---|---|---|---|
| | (33795) | (38883) | (38827) |
| Peptide | MIC (μM) | MIC (μM) | MIC (μM) |
| MtDef4 ¹ | 1.1 | 1.1 | 0.55 |
| GMA4C_V1A | 11.2 | 11.2 | 11.2 |
| (SEQ ID NO: 3) | | | |
| GMA4C_V3A | 2.8 | 5.6 | 2.8 |
| (SEQ ID NO: 5) | | | |

¹ Full Length MtDef4 protein is described in U.S. Pat. No. 7,825,297.

All pathogen isolates used in this assay are resistant to an antifungal drug Fluconazole. Antifungal assays were conducted in half-strength potato dextrose broth.

TABLE 12
Synergistic (or Additive) Effects of
GMA4C_v3 or MtDef4/Fluconazole Combination

| | GMA4C_V3A (SEQ ID NO: 5 )/ Fluconazole | | | |
|---|---|---|---|---|
| | MIC | MIC in | | |
| | alone | combination | | |
| | μg/ml | μg/ml | | |
| | (μM) | (μM) | FICI¹ | Interpretation |
| C. albicans | 11.7/64 | 2.9/8 | 0.375 | Synergistic |
| | (4.2/206) | (1.4/25.8) | | |
| C. glabrata | 11.7/64 | 5.9/8 | 0.625 | Additive |
| | (4.2/206) | (2.8/25.8) | | |

Full Length MtDef4² Protein/ Fluconazole

| | MIC | MIC in | | |
|---|---|---|---|---|
| | alone | combination | | |
| | μg/ml | μg/ml | | |
| | (μM) | (μM) | FICI¹ | Interpretation |
| C. albicans | 5.9/64 | 2.9/2 | 0.53 | Additive |
| | (1.1/206) | (0.55/6.45) | | |
| C. glabrata | 2.9/64 | 1.5/64 | 1.5 | No |
| | (1.1/206) | (0.28/206) | | difference |

Conclusion: GMA4C_v3 shows synergistic enhancement of antifungal activity against drug resistant C. albicans in combination with Fluconazole
¹FICI is Fractional Inhibitory Concentration Index. It is calculated as MIC_{A} combination/MIC alone + MIC_{B} combination/MIC_{B} alone where MIC_{A} combination is the MIC of agent A in combination and MIC_{A} alone is the MIC of agent A alone). Agent A is peptide (GMAC_V3A) and Agent B is Fluconazole in top table. Agent A is full length MtDef4 protein and Agent B is Fluconazole in fluconazole in bottom table.
²Full Length MtDef4 protein is described in U.S. Pat. No. 7,825,297, incorporated herein by reference in its entirety.

TABLE 13
Antifungal activity of peptides (MIC in μM)
against human nail pathogens (Tricophyton spp.)
and Aspergillosis (Aspergillus fumigatus).

| | Tricophyton | Tricophyton | Aspergillus |
|---|---|---|---|
| Peptide | rubrum | metagrophytes | fumigatus |
| (SEQ ID NO:) | (23014) | (26103) | (36843) |
| MtDef4 ¹ | 2.2 | 1.1 | >8.9 |
| GMA4C_V1A | 11.2 | 11.2 | 22.4 |
| (SEQ ID NO: 3) | | | |
| GMA4C_V3A | 5.6 | 5.6 | 11.2 |
| (SEQ ID NO: 5) | | | |
| OeDef1 ² | 3.8 | 3.8 | >7.6 |
| OeDef1_V3 | 4.0 | 4.0 | >7.9 |
| (SEQ ID NO: 42) | | | |

¹ Full Length MtDef4 protein is described in U.S. Pat. No. 7,825,297.
² Full length OeDefl defensin protein is described in WO 2020/146373.

TABLE 14
Antifungal activity of GMA4C variants (MIC in μM) against
Candida spp. and Aspergillosis (Aspergillus fumigatus).

| | | GMA4C_V1A | GMA4C_V2A | GMA4C_V4 | GMA4C_V5 |
|---|---|---|---|---|---|
| | | (SEQ ID | (SEQ ID | (SEQ ID | (SEQ ID |
| Species | Isolate | NO: 3) | NO: 4) | NO: 11) | NO: 12) |
| Candida | SC5314 | 3.8 | 7.6 | 3.8 | 7.6 |
| albicans | ATCC | 3.8 | 7.6 | 3.8 | 7.6 |
| | 90028 | | | | |
| | CA3 | 3.8 | 7.6 | 3.8 | 3.8 |
| Candida | ATCC | 1.9 | 3.8 | 3.8 | 3.8 |
| parapsilosis | 22019 | | | | |
| Candida | DI17-47 | 3.8 | 7.6 | 3.8 | 7.6 |
| auris | DI17-48 | 3.8 | 7.6 | 3.8 | 7.6 |
| | DI17-46 | 3.8 | 7.6 | 3.8 | 7.6 |
| Aspergillus | AF293 | 15.2 | 15.2 | 7.6 | 15.2 |
| fumigatus | DI15-106 | >15.2 | >15.2 | 7.6 | >15.2 |
| | DI15-116 | >15.2 | >15.2 | 15.2 | >15.2 |
| Fusarium | F1 (F. | 3.8 | 3.8 | 3.8 | 3.8 |
| | oxysporum) | | | | |
| | F2 (F. | 3.8 | 7.6 | 3.8 | 7.6 |
| | oxysporum) | | | | |
| | F4 (F. | 3.8 | 3.8 | 3.8 | 3.8 |
| | solani) | | | | |
| Coccidioides | Cocci1 | 3.8 | 3.8 | 1.9 | 0.9 |
| sp. | Cocci2 | 1.9 | 7.6 | 7.6 | 7.6 |
| | DI17-143 | 1.9 | 7.6 | 7.6 | 7.6 |

### Example 3. Antibacterial Activity of GMA4C_V1A Against Human Bacterial Pathogens

Salmonella Typhimurium var. Copenhagen, Enterotoxigenic E. coli—F4 and Listeria monocytogens (F5244) strains were grown overnight on an LB agar plate at 37_C. A small number of bacteria was scraped from the plate and added to Mueller-Hinton (MH) growth media and grown to log phase. Cells were diluted to 1-3×10⁶ CFU/mL and 50 ul were added to each well in a polypropylene 96-well plate. Synthesized peptide GMA4C_V1A was diluted into 0.2% BSA, 0.01% acetic acid solution and added to the concentration of 0.2, 0.4, 0.80, 1.6, 3.25, 7.5, 15, 30 and 60 and 120 μM. Then, 50 μl of each peptide solution was added to 50 μl of bacterial cells. Plates were parafilmed and incubated at 37° C. overnight. The concentrations at which bacterial growth inhibition was determined based on the OD₆₀₀ nm value at each concentration relative to the OD₆₀₀ nm value of MH medium alone and that of bacterial growth in MH medium without any peptide. The Minimal Inhibitory Concentration (MIC) is the concentration of the peptide at which there is no significant growth of the microorganism relative to the growth of the microorganism in growth medium lacking the peptide.

TABLE 15
Antibacterial activity of GMA4C_V1A (SEQ ID NO: 3)
Concentrations of GMA4C_V1A (in μM) at which growth
inhibition of each bacterial pathogen was observed

| | S. Typhimurium var. | Enterotoxigenic | L. monocytogenes |
|---|---|---|---|
| Peptide | Copenhagen | E. coli-F4 | F5244 |
| GMA4C V1 | 15-60 | 7.5-60 | 15-30 |

### Example 4. Antimicrobial Activity of Defensin Peptide Fragments

Crude chemically synthesized defensin-derived peptides with 80-85% purity were obtained from Biomatik Inc, Canada or from Alan Scientific Inc., USA. Each peptide was further purified using a C-18 reverse phase HPLC (Agilent, Singapore). HPLC fractions containing the peptide were lyophilized and resuspended in nuclease-free water. Concentrations were determined using BCA assays using manufacturer's protocol (Thermo-Fisher Scientific) for their accurate quantification.

The fungal strains of Botrytis cinerea T-4, Alternaria alternata, Cercospora sojina and, Colletotrichum gloeosporioides were each cultured in their respective normal growth medium shown in Table 16. Fungal spores were harvested by flooding the fungal growth plates with sterile water. The spore suspension was filtered through two layers of Miracloth, centrifuged at 13,600 rpm for 1 min, washed, and re-suspended in low-salt Synthetic Fungal Medium (SFM) (U.S. Pat. No. 6,316,407). The spore suspension was adjusted to the desired spore count using a hemocytometer.

TABLE 16

| | Medium for spore | Culture |
|---|---|---|
| Strains | production | conditions |
| B. cinerea | 20% V8 | 7-25 days, 25° C. |
| Alternaria | 10% Potato-Dextrose Agar | 7-15 days, 25° C. |
| alternata | | |
| Cercospora | 20% V8 | 12 hr light/12 hr |
| sojina | | dark at 25° C. |
| Colletotrichum | 10% Potato-Dextrose Agar | 7-15 days, 25° C. |
| gloeosporioides | | |

The antifungal activity of truncated defensin-derived peptides and their variants against B. cinerea, A. alternata, C. sojina and C. gloeosporioides fungal pathogens was determined spectrophotometrically using a 96-well plate assay (Sagaram et al., (2011) PLoS ONE 6: e18550. doi:10.1371/journal.pone.0018550). Forty-five microliter of peptide at concentrations of 0.375, 0.75, 1.5, 3, 12 μM was added to each well of the microtiter plate containing 45 μL of (˜10⁵ B. cinerea spores/ml) spore suspension. The quantitative fungal growth inhibition was determined by measuring the absorbance at 595 nm using a (Tecan Infinite M200 ProTecan Systems Inc., San Jose, CA) microplate reader after 48 h. Fungal cell viability was determined using the resazurin cell viability assay (Chadha and Kale, (2015) Lett Appl Microbiol 61, 238-244, Li et al., (2019) Mol Plant Microbe Interact 32, 1649-1664). After incubation of the pathogen/peptide mixture for 48 h, 10 μl of 0.1% resazurin solution was added to each well and re-incubated overnight. A change in color of the resazurin from blue to pink or colorless indicated the presence of live fungal cells. The MIC value of each peptide was determined as the minimal concentration of peptide at which no change in blue color was observed. MIC values of defensin-derived peptides and their variants were also determined in SFM and SFM supplemented with 100 mM NaCl, 100 mM or 2 mM CaCl₂) as described above.

The semi-in planta antifungal activity of each defensin-derived peptide and its variants against B. cinerea was determined using the detached leaves of N. benthamiana Nb1 as described previously (Li et al., (2019) Mol Plant Microbe Interact 32, 1649-1664; Velivelli et al., (2020) Proceedings of the National Academy of Sciences. 117, 16043-16054). Each peptide was tested at concentrations of 1.5 μM, 3 μM and 6 μM. Following incubation of each peptide/fungal spore mixture at room temperature for 48 h, leaves were photographed in white-light. High-resolution fluorescence images were also taken using CropReporter (PhenoVation, Wageningen, Netherlands). These images depicted the calculated F_{V}/F_{M} (maximum quantum yield of photosystem II) values of diseased area affected by B. cinerea infection. Colors in the images show five different classes ranging from class I to class V (0.000 to 0.700) depicting varying degrees of tissue damage. Green color in each image represents class V in 0.600 to 0.700 range depicting healthy area on leaf surface. In contrast, red color represents class I in the 0.000 to 0.160 range and depicts severely damage or diseased leaf surface.

The primary amino acid sequences, length, net charge and percentage of hydrophobic amino acids of defensin-derived peptides are shown in Table 17. These peptides are derived from plant defensins OeDef1, MtDef4, MsDef1 and MtDef5A. Amino acid substitutions in the wild-type sequence of each peptide were made to increase the net charge and hydrophobicity. In addition, a disulfide bond was introduced into specific variants to make them pseudo-cyclic. Peptides that can form a single disulfide bond and a pseudo-cyclic peptide are shown as “+” in the “Disulfide Bond” column of Table 17. All peptides also carry a carboxy-terminal amide group.

TABLE 17
Amino acid sequences, length, net charge and % hydrophobic amino acids of
defensin-derived peptides. The presence of a
disulfide bond is indicated where necessary.

| | | SEQ | | Net | % |
|---|---|---|---|---|---|
| Peptide | Sequence (disulfide presence) | ID NO | Length | Charge | Hydrophob. |
| GMAOe1C | GACLKNRHSKHYGCYCYYRHCY- | 577 | 22 | 4.5 | 32 |
| (WT) | NH₂ | | | | |
| GMAOe1C_V3 | GACLKNRHSKHYGWFWYYRHCY- | 578 | 22 | 4.5 | 41 |
| | NH₂ (disulfide +) | | | | |
| GMAOe1C_V4 | GACLKNRHSKHYGFFWYYRHCY- | 579 | 22 | 4.5 | 41 |
| | NH₂ (disulfide +) | | | | |
| GMA4AC | GRCRGFRRRCFCTTHC-NH₂ | 580 | | | |
| GMA4C_V9 | GGRCKGFLRRFWFTRIC-NH₂ | 581 | 17 | 5 | 35 |
| | (disulfide +) | | | | |
| GMA4C_V10 | GGRCKGFRRRWYWTRIC-NH₂ | 582 | 17 | 6 | 29 |
| | (disulfide +) | | | | |
| GMA1C_V1 | SGRCRILFRCFCTKNC-NH₂ | 583 | 16 | 4 | 25 |
| GMA1C_V2 | SGRCRILFRWFWTKNC-NH₂ | 584 | 16 | 4 | 37 |
| | (disulfide +) | | | | |
| GMA1C_V3 | SGRCRILFRWYFTKNC-NH₂ | 585 | 16 | 4 | 37 |
| | (disulfide +) | | | | |
| GMA5CA_WT | GACHRQGFGFACFCYKKC-NH₂ | 586 | 18 | 3.5 | 28 |
| GMA5CA_V3 | GACHRQGFGFAWFWYKKC-NH₂ | 19 | 18 | 3.5 | 39 |
| | (disulfide +) | | | | |
| GMA5CA_V4 | GACHRQGFGFAFFFYKKC-NH₂ | 20 | 18 | 3.5 | 39 |
| | (disulfide +) | | | | |

The minimal inhibitory concentration (MIC) value of each defensin-derived peptide and its variants was determined (Table 18). It has been hypothesized that the presence of cations significantly weakens the electrostatic interactions between a positively charged defensin and negatively charged fungal membranes (Chu et al., (2013) Antimicrobial Agents and Chemotherapy 57: 4050-4052). We therefore determined the antifungal activity of each peptide against B. cinerea in SFM supplemented with 100 mM NaCl, 100 mM KCl or 2 mM CaCl₂) (Table 18).

TABLE 18
MIC values of defensin-derived peptides against Botrytis cinerea

| | | | MIC (μM) | MIC (μM) | MIC (μM) |
|---|---|---|---|---|---|
| | SEQ | | (SFM + | (SFM + | (SFM + |
| | ID | MIC (μM) | 100 mM | 100 mM | 2 mM |
| Peptide | NO | SFM | NaCl) | KCl) | CaCl₂) |
| GMAOe1C_WT | 577 | 3 | 3-6 | 6-12 | >12 |
| GMAOe1C_V3 | 578 | 1.5 | 2-3 | 6-12 | 3-6 |
| GMAOe1C_V4 | 579 | 1.5 | 6 | 6-12 | 6-12 |
| GMA4AC | 580 | 3 | ND¹ | ND | ND |
| GMA4C_V9 | 581 | 3 | 3 | 6-12 | 12 |
| GMA4C_V10 | 582 | 3 | 3 | 3-6 | 3 |
| GMA1C | 35 | >12 | >12 | >12 | >12 |
| GMA1C_V1 | 583 | >6 | >6 | >12 | — |
| GMA1C_V2 | 584 | 1.5-3 | 3 | >12 | 3 |
| GMA1C_V3 | 585 | 1.5 | 3 | >12 | 6 |
| GMA5CA_WT | 586 | 6 | >12 | >12 | >12 |
| GMA5CA_V3 | 19 | 3 | >12 | >12 | >12 |
| GMA5CA_V4 | 20 | 3 | >12 | >12 | >12 |

¹ND is not determined.

MIC values of defensin-derived peptides were also tested against Alternaria alternata, Cercospora sojina, and Colletotrichum gloeosporioides (Table 19).

TABLE 19
In vitro antifungal activity of antifungal peptides against Alternaria
alternata, Cercospora sojina and Colletotrichum gloeosporioides

| | SEQ | Alternaria | Cercospora | Colletotrichum |
|---|---|---|---|---|
| Peptide | ID NO | alternata | sojina | gloeosporioides |
| GMA4AC_WT | 580 | >12 | >12 | >12 |
| GMA 4C_V9 | 581 | >12 | 6 | >12 |
| GMA 4C_V10 | 582 | >12 | 6 | >12 |
| GMA5CA_WT | 586 | 6 | 12 | >12 |
| GMA5CA_V3 | 19 | 3 | 6 | >12 |
| GMA5CA_V4 | 20 | 3 | 6 | >12 |

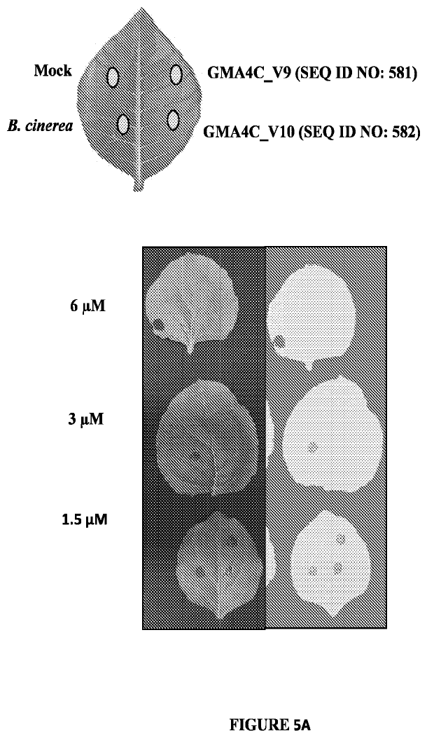

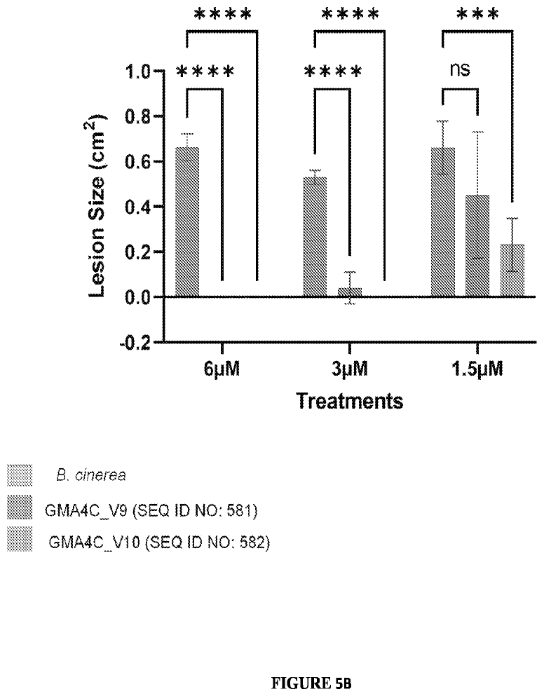

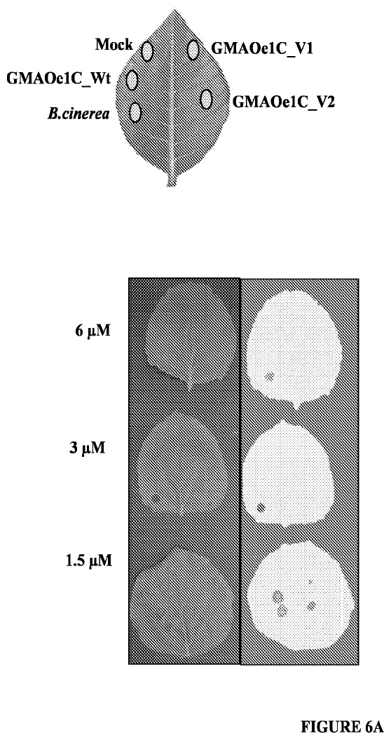

Semi-in planta antifungal activity of GMA4C_V9, GMA4C_V10, GMAOe1C_WT, GMAOe1C_V3, GMAOe1C_V4, GMA1C_V1 and GMA1C_V2 peptides against B. cinerea was determined by using the detached leaves of N. benthamiana. Each peptide at concentrations of 1.5 μM, 3 μM and 6 μM was applied on leaf as drops and freshly prepared conidial inoculum was applied immediately to each drop of the peptide. Leaves were assessed for attenuation of gray mold symptoms after 48 h of inoculation relative to no peptide controls by measuring lesion sizes. GMA4C_V9 and GMA4C_V10 peptides are both effective in reducing gray mold disease symptoms. However, at low concentrations of 1.5 μM and 3 μM, GMA4C_V10 is more effective in reducing the symptoms of gray mold than GMA4C_V9 (FIG. 5).

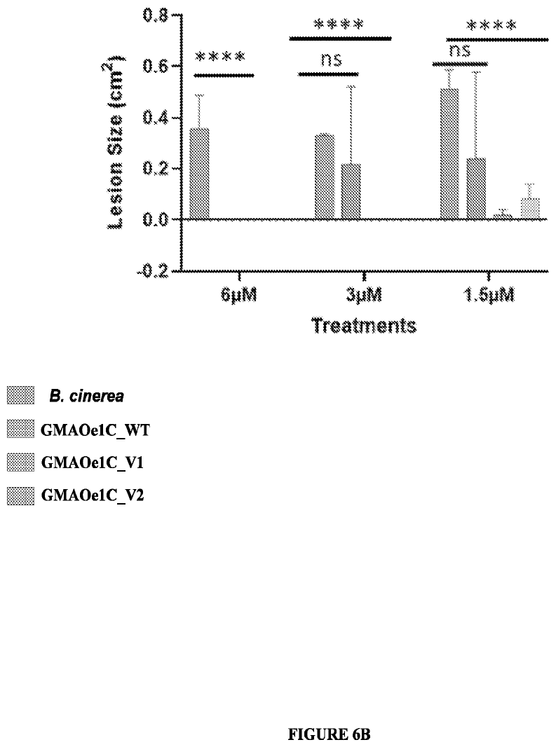

A drop inoculation assay was also performed to test the antifungal activity of GMAOe1C_WT, GMAOe1C_V1, GMAOe1C_V2. The results revealed that GMAOe1C_V1 and GMAOe1C_V2 at concentration of 3 and 6 μM completely abrogated gray mold symptoms, but GMAOe1C_WT was only effective at 6 μM. At a concentration of 1.5 μM, GMAOe1C_V3 is more effective than GMAOe1C_V4 or GMAOe1C_WT (FIG. 6).

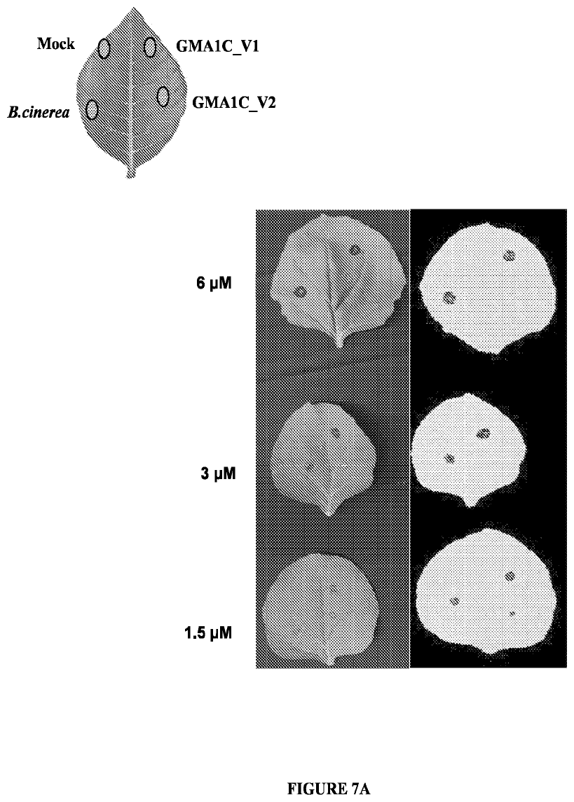

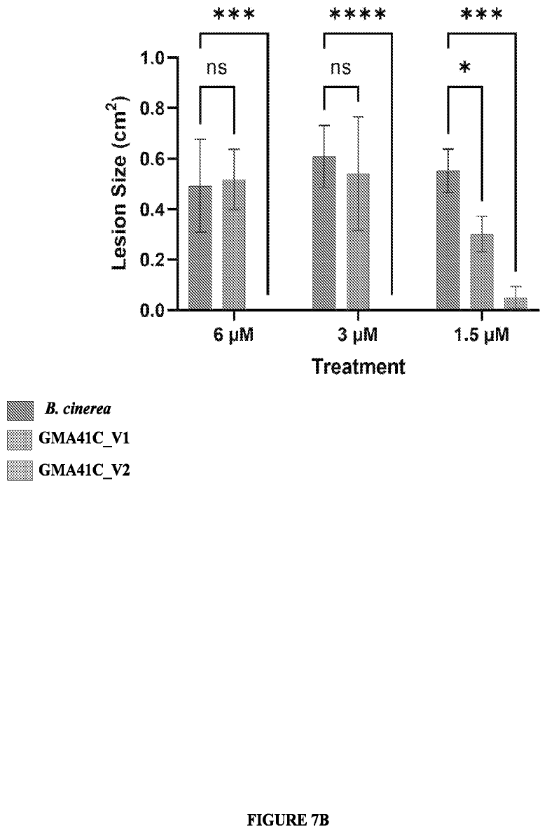

A drop inoculation assay was also performed to test the antifungal activity of GMA1C_V1 and GMA1C_V2. (FIG. 6). GMA1C_V2 at 3 μM and 6 μM completely abrogated gray mold symptoms. GMA1C_V1 failed to reduce disease symptoms at these concentrations. At a concentration of 1.5 μM, GMA1C_V2 was more effective than GMA1C_V1 (FIG. 7).

The results above indicate that the panel of modifications (e.g., amino acid substitutions) introduced into these truncated defensin-derived peptides confer greater antifungal activity than the wild-type truncated peptides.

The breadth and scope of the present disclosure should not be limited by any of the above-described examples.

What is claimed is:

1. A peptide comprising the amino acid sequence of:
(a) a modified gamma-core consensus sequence GXCX3-10(F/W/Y/L/V/I/M) (F/W/Y)(F/W/Y/L/V/I/M) (SEQ ID NO: 50), GXCX3-10(F/W/Y)(F/W/Y)(F/W/Y) (SEQ ID NO: 587), GXCX3-8 (F/W/Y)(F/W/Y)(F/W/Y) (SEQ ID NO: 33), GXCX3-9(F/W/Y)(F/W/Y)(F/W/Y) (SEQ ID NO: 34), GXCX3-8 (F/W/Y) (SEQ ID NO: 43), GXCX3-9(F/W/Y) (SEQ ID NO: 44), GXCX3-8(F/W/Y/L/V/I/M) (SEQ ID NO: 45), GXCX3-10(F/W/Y/L/V/I/M) (SEQ ID NO: 46), GXCX3-8(L/V/I/M)(F/W/Y)(L/V/I/M) (SEQ ID NO: 47), GXCX3-10(L/V/I/M)(F/W/Y)(L/V/I/M) (SEQ ID NO: 48), GXCX3-8(F/W/Y/L/V/I/M) (F/W/Y)(F/W/Y/L/V/I/M) (SEQ ID NO: 49), GXCX3-8(F/W/Y/L/V/I/M)(F/W/Y/L/V/I/M)(F/W/Y/L/V/I/M) (SEQ ID NO: 51), or GXCX3-10(F/W/Y/L/V/I/M)(F/W/Y/L/V/I/M)(F/W/Y/L/V/I/M) (SEQ ID NO: 52), wherein the peptide further comprises a second C-terminal cysteine residue located C-terminal to the cysteine residue in the modified gamma-core consensus sequence, and wherein the peptide has a net positive charge of at least 3 and a hydrophobic amino acid content at least 18%; or
(b) SEQ ID NO: 1, wherein said peptide does not comprise the corresponding full-length sequence of the defensin peptide of SEQ ID NO: 8, 16, 20, 23, 26, 29, 37, 38, 39, 40, or 41; optionally wherein said peptide comprises a modified gamma-core consensus sequence GXCX3-8 (F/W/Y)(F/W/Y)(F/W/Y) (SEQ ID NO: 33), GXCX3-9(F/W/Y)(F/W/Y)(F/W/Y) (SEQ ID NO: 34), GXCX3-8 (F/W/Y) (SEQ ID NO: 43), or GXCX3-9(F/W/Y) (SEQ ID NO: 44); and/or optionally wherein the C-terminal cysteine residue or C-terminal amino acid residue is amidated.

2. The peptide of claim 1, wherein said peptide comprises (i) conserved C1 and C4 cysteine residues corresponding to N-terminal and C-terminal cysteines of a reference defensin C-terminal peptide, wherein amino acid residues 16 and 18 corresponding to conserved C2 and C3 cysteine residues of the reference defensin C-terminal peptide are independently substituted with tryptophan, tyrosine, phenylalanine, leucine, valine, isoleucine, or methionine; and optionally wherein the defensin peptide variant has a net positive charge of at least 3 and a hydrophobic amino acid content at least 18%; or (ii) an amino acid sequence having at least 50%, 55%, 60%, 68%, 75%, 82%, or 94% sequence identity across the entire length of SEQ ID NO:7, SEQ ID NO: 12, SEQ ID NO: 13, SEQ ID NO: 14, 15, 17-20, 22, 23, 25, 26, 28, 29, 31, 32, 42, 578, 579, 581, 582, 584, or 585, wherein said peptide does not comprise the corresponding full-length sequence of the defensin peptide of SEQ ID NO: 8, 16, 20, 23, 26, 29, 37, 38, 39, 40, or 41; and optionally wherein the peptide of (i) or (ii) comprises a disulfide bond between the two cysteine residues.

3. The peptide of claim 1 or 2, wherein the peptide comprises, essentially consists, or consists of: (i) 30 amino acid residues or less; or (ii) 15, 16, or 17 to 30 amino acid residues.

4. A composition comprising the peptide of claim 1 or 2 and an agriculturally, pharmaceutically, or veterinary-practicably acceptable carrier, diluent, or excipient.

5. The composition of claim 4, wherein the peptide is provided at a concentration of about 0.1, 0.5, 1.0, or 5 pg/ml to about 1, 5, 20, 50, or 100 mg/ml or at a concentration of about 0.1, 0.5, 1.0, or 5 pg/gram to about 1, 5, 20, 50, or 100 mg/gram and optionally wherein the composition comprises a sodium salt at a concentration of at least 100 mM and/or a calcium salt at a concentration of at least 2 mM.

6. A method for preventing or reducing crop damage by a plant pathogenic microbe comprising the step of contacting a plant, a plant seed, or other part of said plant with an effective amount of the composition of claim 4.

7. The method of claim 6, wherein the plant pathogenic microbe is a Fusarium sp., Alternaria sp., Verticillium sp., Phytophthora sp., Colletotrichum sp., Botrytis sp., Cercospora sp., Phakopsora sp. Rhizoctonia sp., Sclerotinia sp., Pythium sp., Phoma sp., Leptosphaeria sp., Gaeumannomyces sp., Puccinia sp. Septoria sp., Penicillium sp., Lasiodiplodia sp., Phomopsis sp., Mycosphaerella sp., Golovinomyces sp., Erisyphe sp., Albugo sp., Setosphaeria sp., Cochiobolus sp., Helminthosporium sp., Diplodia sp., or Stenocarpella sp.

8. A medical device comprising the device and the composition of claim 4, wherein the device comprises at least one surface that is topically coated and/or impregnated with the composition.

9. The medical device of claim 8, wherein said device is a stent, a catheter, a contact lens, a condom, a patch, or a diaphragm.

10. A method for treating, preventing, or inhibiting a microbial infection in a subject in need thereof comprising administering to said subject an effective amount of the composition of claim 4.

11. The method of claim 10, wherein said administration comprises topical, enteral, parenteral, and/or intravenous introduction of the composition.

12. The method of claim 10, wherein the subject is a human, livestock, poultry, fish, or a companion animal.

13. The method of claim 10, wherein the microbial infection is of a mucosal membrane, eye, skin, and/or a nail and the composition is applied to the mucosal membrane, eye, skin, and/or nail.

14. The method of claim 10, wherein the microbial infection is by a dermatophyte, and wherein the dermatophyte is optionally selected from the group consisting of Trichophyton rubrum, Trichophyton interdigitale, Trichophyton violaceum, Trichophyton tonsurans, Trichophyton soudanense, Trichophyton mentagrophytes, Microsporum flavum, Epidermophyton floccosum, and Microsporum gypseum.

15. The method of claim 10, wherein the microbial infection is by an Aspergillus, Cryptococcus, Penicillium, Rhizopus, Apophysomyces, Cunninghamella, Saksenaea, Rhizomucor, Syncephalostrum, Cokeromyces, Actinomucor, Pythium, Fusarium, Histoplasmosis, or Blastomyces species.

16. The method of claim 10, wherein the microbial infection is by a Candida species and wherein the Candida species is Candida albicans (C. albicans), C. auris, C. glabrata, C. parasilosis, C. tropicalis, or C. krusei.

17. The composition of claim 4 for use in a method of treating, preventing, or inhibiting microbial infection in a subject in need thereof.

18. The composition of claim 17, wherein the subject is a human, livestock, poultry, fish, or a companion animal.

19. A plant part that is at least partly coated with the composition of claim 4.

20. The plant part of claim 17, wherein the part is a seed and the seed is optionally a corn, soybean, wheat, rice, cotton, Brassica sp., or tomato seed.

21. The plant part of claim, wherein the plant part is a fruit, vegetable, or flower.

22. A recombinant polynucleotide comprising a polynucleotide encoding a peptide comprising the peptide of claim 1 or 2, wherein the polynucleotide encoding the first antimicrobial peptide is operably linked to a polynucleotide comprising a promoter which is heterologous to the polynucleotide encoding the first antimicrobial peptide, optionally wherein any amino acid substitution in said sequence increases or maintains the net positive charge and/or increases or maintains hydrophobicity of the peptide.

23. The recombinant polynucleotide of claim 22, wherein the recombinant polynucleotide further comprises a polynucleotide encoding: (i) a transit peptide, a vacuolar targeting peptide, and/or an endoplasmic reticulum targeting peptide; (ii) a plastid targeting peptide; and/or (iii) a polyadenylation or transcriptional termination signal, wherein the polynucleotides of (i), (ii), and/or (iii) are operably linked to the polynucleotide encoding the antimicrobial peptide.

24. The recombinant polynucleotide of claim 22, wherein the polynucleotide encoding the first antimicrobial peptide is inserted into a heterologous nuclear or plastid genome of a cell and operably linked to an endogenous promoter located in the heterologous nuclear or plastid genome.

25. A plant nuclear or plastid genome comprising a polynucleotide encoding a peptide comprising the peptide of claim 1 to 2, wherein the polynucleotide is heterologous to the nuclear or plastid genome and wherein the polynucleotide is operably linked to an endogenous promoter of the nuclear or plastid genome.

26. A cell comprising the recombinant polynucleotide of claim 22, wherein the cell is optionally a bacterial, yeast, or plant cell.

27. A plant comprising the recombinant polynucleotide of claim 22

28. A plant part of the plant of claim 27, wherein the plant part comprises the recombinant polynucleotide, optionally wherein the plant part is a seed, stem, leaf, root, tuber, flower, or fruit.

29. A method for producing plant seed that provides plants resistant to infection by a plant pathogenic microbe that comprises the steps of: (i) selfing or crossing the plant of claim 27; and (ii) harvesting seed that comprises the recombinant polynucleotide of the plant from the self or cross, thereby producing plant seed that provide plants resistant to infection by a plant pathogenic microbe.


SEQUENCE LISTING (591 sequences)

SEQ ID NO: 1 - protein, 23 aa, Artificial
  synthetic
  VARIANT (1)..(1): Xaa is F, G, or absent
  VARIANT (3)..(3): Xaa is A, G, Y, H, S, R or diaminobutyric acid
  VARIANT (5)..(5): Xaa is H, L, S, N, R, K, or diaminobutyric acid
  VARIANT (6)..(15): Xaa is R, K, H, N, D, F, G, Q, P, A, I, L, V, Y, S, or diaminobutyric acid, , wherein one or more of Xaa 8, Xaa 9, Xaa 10, Xaa 11, Xaa 12, Xaa 13, Xaa 14, and/or Xaa 15 may be absent
  VARIANT (16)..(16): Xaa is C, V, W, Y, or F
  VARIANT (17)..(17): Xaa is L, I, W, Y, or F
  VARIANT (18)..(18): Xaa is C, V, W, Y, or F
  VARIANT (19)..(19): Xaa is K, T, F, W, or Y
  VARIANT (20)..(20): Xaa is R, K, T, L, F, Q, or diaminobutyric acid
  VARIANT (21)..(21): Xaa is N, H, K, R, P, Q, I, F, W, or Y
  VARIANT (22)..(22): Xaa is N, H, K, P, Q, I, F, W, Y, or absent
  MISC_FEATURE (23)..(23): C is optionally amidated
XGXCXXXXXXXXXXXXXXXXXXC

SEQ ID NO: 2 - protein, 17 aa, Artificial
  synthetic
  MOD_RES (17)..(17): AMIDATION
GGRCRGFRRRCFCTTHC

SEQ ID NO: 3 - protein, 17 aa, Artificial
  synthetic
  MOD_RES (17)..(17): AMIDATION
GGRCKGFRRRCFCTRIC

SEQ ID NO: 4 - protein, 17 aa, Artificial
  synthetic
  MOD_RES (17)..(17): AMIDATION
GGRCRGFRRRCFCTRIC

SEQ ID NO: 5 - protein, 17 aa, Artificial
  synthetic
  MOD_RES (17)..(17): AMIDATION
GGRCRGFRRRVFVTRIC

SEQ ID NO: 6 - protein, 17 aa, Artificial
  synthetic
  MOD_RES (17)..(17): AMIDATION
FGRCRGFRRRCFCWRWC

SEQ ID NO: 7 - protein, 17 aa, Artificial
  synthetic
  VARIANT (3)..(3): Xaa is diaminobutyric acid
  VARIANT (5)..(5): Xaa is diaminobutyric acid
  VARIANT (8)..(10): Xaa is diaminobutyric acid
  VARIANT (15)..(15): Xaa is diaminobutyric acid
  MOD_RES (17)..(17): AMIDATION
FGXCXGFXXXCFCWXWC

SEQ ID NO: 8 - protein, 16 aa, Artificial
  synthetic
GRCRGFRRRCFCTTHC

SEQ ID NO: 9 - protein, 13 aa, Artificial
  synthetic
  VARIANT (2)..(2): Xaa is any amino acid
  VARIANT (4)..(12): Xaa is any amino acid, wherein Xaa 7 to Xaa 12 are optionally present
GXCXXXXXXXXXC

SEQ ID NO: 10 - protein, 14 aa, Artificial
  synthetic
  VARIANT (2)..(2): Xaa is any amino acid
  VARIANT (4)..(13): Xaa is any amino acid, wherein Xaa 7 to Xaa 13 are optionally present
GXCXXXXXXXXXXC

SEQ ID NO: 11 - protein, 17 aa, Artificial
  synthetic
FGRCRGFRRRCFCWRWC

SEQ ID NO: 12 - protein, 17 aa, Artificial
  synthetic
  VARIANT (3)..(3): Xaa is diaminobutyric acid
  VARIANT (5)..(5): Xaa is diaminobutyric acid
  VARIANT (8)..(10): Xaa is diaminobutyric acid
  VARIANT (15)..(15): Xaa is diaminobutyric acid
FGXCXGFXXXCFCWXWC

SEQ ID NO: 13 - protein, 17 aa, Artificial
  synthetic
  MOD_RES (17)..(17): AMIDATION
GGRCKGFRRRWFWTRIC

SEQ ID NO: 14 - protein, 17 aa, Artificial
  synthetic
  VARIANT (1)..(1): Xaa1 is F, G, or absent
  VARIANT (3)..(3): Xaa is any amino acid
  VARIANT (5)..(5): Xaa is K or R
  VARIANT (11)..(13): Xaa is F, W, or Y
  VARIANT (14)..(14): Xaa is T, F, W, or Y
  VARIANT (15)..(15): Xaa is R, K, or diaminobutyric acid
  VARIANT (16)..(16): Xaa is is I, F, W, or Y
  MISC_FEATURE (17)..(17): Cysteine is optionally amidated
XGRCXGFRRRXXXXXXC

SEQ ID NO: 15 - protein, 17 aa, Artificial
  synthetic
  MOD_RES (17)..(17): AMIDATION
GGRCKGFRRRWYWTRIC

SEQ ID NO: 16 - protein, 18 aa, Artificial
  synthetic
GACHRQGFGFACFCYKKC

SEQ ID NO: 17 - protein, 19 aa, Artificial
  synthetic
  VARIANT (1)..(1): Xaa is F, G, or absent
  VARIANT (3)..(3): Xaa is A, F, W, Y, or absent
  VARIANT (5)..(5): Xaa is is H, R, or K
  VARIANT (6)..(6): Xaa is R or K
  VARIANT (7)..(7): Xaa is Q, R, or K
  VARIANT (10)..(10): Xaa is G, R, or K
  VARIANT (11)..(11): Xaa is F, W, Y, R, or K
  VARIANT (12)..(12): Xaa is A, F, W, Y, R, or K
  VARIANT (13)..(15): Xaa is F, W, or Y
  VARIANT (16)..(16): Xaa is F, W, Y, R, or K
  VARIANT (17)..(18): Xaa is R or K
  MISC_FEATURE (19)..(19)
XGXCXXXGFXXXXXXXXXC

SEQ ID NO: 18 - protein, 18 aa, Artificial
  synthetic
  VARIANT (12)..(14): Xaa is F, W, or Y
  MISC_FEATURE (18)..(18): Cysteine is optionally amidated
GACHRQGFGFAXXXYKKC

SEQ ID NO: 19 - protein, 18 aa, Artificial
  synthetic
  MOD_RES (18)..(18): AMIDATION
GACHRQGFGFAWFWYKKC

SEQ ID NO: 20 - protein, 18 aa, Artificial
  synthetic
  MOD_RES (18)..(18): AMIDATION
GACHRQGFGFAFFFYKKC

SEQ ID NO: 21 - protein, 17 aa, Artificial
  synthetic
  MOD_RES (17)..(17): AMIDATION
GGRCRGFRRRCFCTRPC

SEQ ID NO: 22 - protein, 17 aa, Artificial
  synthetic
  VARIANT (11)..(13): Xaa is F, W, or Y
  MISC_FEATURE (17)..(17): Cysteine is optionally amidated
GGRCRGFRRRXXXTRPC

SEQ ID NO: 23 - protein, 17 aa, Artificial
  synthetic
  MOD_RES (17)..(17): AMIDATION
GGRCRGFRRRWFWTRPC

SEQ ID NO: 24 - protein, 22 aa, Artificial
  synthetic
GACLKNRHSKHYGCYCYYRHCY

SEQ ID NO: 25 - protein, 22 aa, Artificial
  synthetic
  VARIANT (14)..(16): Xaa is F, W, or Y
  MISC_FEATURE (22)..(22): Tyrosine is optionally amidated
GACLKNRHSKHYGXXXYYRHCY

SEQ ID NO: 26 - protein, 22 aa, Artificial
  synthetic
  MOD_RES (22)..(22): AMIDATION
GACLKNRHSKHYGWFWYYRHCY

SEQ ID NO: 27 - protein, 17 aa, Artificial
  synthetic
GGLCRGFRRRCFCTKHC

SEQ ID NO: 28 - protein, 17 aa, Artificial
  synthetic
  VARIANT (11)..(13): Xaa is F, W, or Y
  MISC_FEATURE (17)..(17): Cysteine is optionally amidated
GGLCRGFRRRXXXTKHC

SEQ ID NO: 29 - protein, 17 aa, Artificial
  synthetic
  MOD_RES (17)..(17): AMIDATION
GGLCRGFRRRWFWTKHC

SEQ ID NO: 30 - protein, 17 aa, Artificial
  synthetic
GGYCSSRRQICKCTLQC

SEQ ID NO: 31 - protein, 17 aa, Artificial
  synthetic
  VARIANT (11)..(13): Xaa is F, W, or Y
  MISC_FEATURE (17)..(17): Cysteine is optionally amidated
GGYCSSRRQIXXXTLQC

SEQ ID NO: 32 - protein, 17 aa, Artificial
  synthetic
  MOD_RES (17)..(17): AMIDATION
GGYCSSRRQIWFWTLQC

SEQ ID NO: 33 - protein, 14 aa, Artificial
  synthetic
  VARIANT (2)..(2): Xaa is any amino acid
  VARIANT (4)..(11): Xaa is any amino acid, Xaa7 to Xaa11 are optionally present
  VARIANT (12)..(14): Xaa is F, W, or Y
GXCXXXXXXXXXXX

SEQ ID NO: 34 - protein, 15 aa, Artificial
  synthetic
  VARIANT (2)..(2): Xaa is any amino acid
  VARIANT (4)..(12): Xaa is any amino acid, Xaa7 to Xaa12 are optionally present
  VARIANT (13)..(15): Xaa is F, W, or Y
GXCXXXXXXXXXXXX

SEQ ID NO: 35 - protein, 15 aa, Artificial
  synthetic
GRCRDDFRCWCTKNC

SEQ ID NO: 36 - protein, 15 aa, Artificial
  synthetic
GRCRILFRCFCTKNC

SEQ ID NO: 37 - protein, 16 aa, Artificial
  synthetic
GHCSKILRRCLCTKPC

SEQ ID NO: 38 - protein, 18 aa, Artificial
  synthetic
GSCNYVFPAHKCICYFPC

SEQ ID NO: 39 - protein, 18 aa, Artificial
  synthetic
GACHVRNGKHMCFCYFNC

SEQ ID NO: 40 - protein, 18 aa, Artificial
  synthetic
GACHYQFPSVKCFCTQNC

SEQ ID NO: 41 - protein, 18 aa, Artificial
  synthetic
GACHRQGIGFACFCKKKC

SEQ ID NO: 42 - protein, 22 aa, Artificial
  synthetic
GACLKNRHSKHYGFFWYYRHCY

SEQ ID NO: 43 - protein, 12 aa, Artificial
  synthetic
  VARIANT (2)..(2): Xaa is any amino acid
  VARIANT (4)..(11): Xaa is any amino acid, Xaa7 to Xaa11 are optionally present
  VARIANT (12)..(12): Xaa is F, W, or Y
GXCXXXXXXXXX

SEQ ID NO: 44 - protein, 13 aa, Artificial
  synthetic
  VARIANT (2)..(2): Xaa is any amino acid
  VARIANT (4)..(12): Xaa is any amino acid, Xaa7 to Xaa12 are optionally present
  VARIANT (13)..(13): Xaa is F, W, or Y
GXCXXXXXXXXXX

SEQ ID NO: 45 - protein, 12 aa, Artificial
  synthetic
  VARIANT (2)..(2): Xaa is any amino acid
  VARIANT (4)..(11): Xaa is any amino acid, Xaa7 to Xaa11 are optionally present
  VARIANT (12)..(12): Xaa is F, W, Y, L, V, I, or M
GXCXXXXXXXXX

SEQ ID NO: 46 - protein, 14 aa, Artificial
  synthetic
  VARIANT (2)..(2): Xaa is any amino acid
  VARIANT (4)..(13): Xaa is any amino acid, Xaa7 to Xaa13 are optionally present
  VARIANT (14)..(14): Xaa is F, W, Y, L, V, I, or M
GXCXXXXXXXXXXX

SEQ ID NO: 47 - protein, 14 aa, Artificial
  synthetic
  VARIANT (2)..(2): Xaa is any amino acid
  VARIANT (4)..(11): Xaa is any amino acid, Xaa7 to Xaa11 are optionally present
  VARIANT (12)..(12): Xaa is  L, V, I, or M
  VARIANT (13)..(13): Xaa is F, W, or Y
  VARIANT (14)..(14): Xaa is  L, V, I, or M
GXCXXXXXXXXXXX

SEQ ID NO: 48 - protein, 16 aa, Artificial
  synthetic
  VARIANT (2)..(2): Xaa is any amino acid
  VARIANT (4)..(13): Xaa is any amino acid, Xaa7 to Xaa13 are optionally present
  VARIANT (14)..(14): Xaa is  L, V, I, or M
  VARIANT (15)..(15): Xaa is F, W, or Y
  VARIANT (16)..(16): Xaa is  L, V, I, or M
GXCXXXXXXXXXXXXX

SEQ ID NO: 49 - protein, 14 aa, Artificial
  synthetic
  VARIANT (2)..(2): Xaa is any amino acid
  VARIANT (4)..(11): Xaa is any amino acid, Xaa7 to Xaa11 are optionally present
  VARIANT (12)..(12): Xaa is F, W, Y, L, V, I, or M
  VARIANT (13)..(13): Xaa is F, W, or Y
  VARIANT (14)..(14): Xaa is F, W, Y, L, V, I, or M
GXCXXXXXXXXXXX

SEQ ID NO: 50 - protein, 16 aa, Artificial
  synthetic
  VARIANT (2)..(2): Xaa is any amino acid
  VARIANT (4)..(13): Xaa is any amino acid, Xaa7 to Xaa13 are optionally present
  VARIANT (14)..(14): Xaa is F, W, Y, L, V, I, or M
  VARIANT (15)..(15): Xaa is F, W, or Y
  VARIANT (16)..(16): Xaa is F, W, Y, L, V, I, or M
GXCXXXXXXXXXXXXX

SEQ ID NO: 51 - protein, 14 aa, Artificial
  synthetic
  VARIANT (2)..(2): Xaa is any amino acid
  VARIANT (4)..(11): Xaa is any amino acid, Xaa7 to Xaa11 are optionally present
  VARIANT (12)..(14): Xaa is F, W, Y, L, V, I, or M
GXCXXXXXXXXXXX

SEQ ID NO: 52 - protein, 16 aa, Artificial
  synthetic
  VARIANT (2)..(2): Xaa is any amino acid
  VARIANT (4)..(13): Xaa is any amino acid, Xaa7 to Xaa13 are optionally present
  VARIANT (14)..(16): Xaa is F, W, Y, L, V, I, or M
GXCXXXXXXXXXXXXX

SEQ ID NO: 53 - protein, 74 aa, Arabidopsis thaliana
CTSTLINGSSDEERTYSFSPTTSPFDPRSLNQELKIGRIGYCFDCARACMRRGKYIRTCSFERKLCRCSISDIK

SEQ ID NO: 54 - protein, 57 aa, Oryza sativa
GTTKVAEARHCLSQSHRFKGMCVSSNNCANVCRTESFPDGECKSHGLERKCFCKKVC

SEQ ID NO: 55 - protein, 108 aa, Artemisia vulgaris
AGSKLCEKTSKTYSGKCDNKKCDKKCIEWEKAQHGACHKREAGKESCFCYFDCSKSPPGATPAPPGAAPPPAAGGSPSPPADGGSPPPPADGGSPPVDGGSPPPPSTH

SEQ ID NO: 56 - protein, 50 aa, Arabidopsis thaliana
RICERRSKTWTGFCGNTRGCDSQCKRWERASHGACHAQFPGFACFCYFNC

SEQ ID NO: 57 - protein, 96 aa, Arabidopsis thaliana
SICNDRLGLCDGCDQRCKAKHGPSCESKCDGPVGMLLCTCTYECGPTKLCNGGLGNCGESCNEQCCDRNCAQRYNGGHGYCNTLDDFSLCLCKYPC

SEQ ID NO: 58 - protein, 46 aa, Arachis duranensis
KLCNHLADTYRGPCFTNASCDDHCKNKEHFVSGTCMKMACWCAHNC

SEQ ID NO: 59 - protein, 57 aa, Malus baccata
RTESSKAIEGKICEFPSTLFKGLCFSSNNCKHTCRKEQFTRGKCSLLTRTCMCTKKC

SEQ ID NO: 60 - protein, 55 aa, Oryza sativa
LAPAQSSVRPCETKSLRFEGFCMVEDNCANVCRTEGFVDGRCSTFVRNCICIKPC

SEQ ID NO: 61 - protein, 45 aa, Triticum dicoccoides
RDCTSQSHKFVGLCLSDRNCASVCLTEYFTGGKCDHRRCVCTKGC

SEQ ID NO: 62 - protein, 47 aa, Pyrus ussuriensis x Pyrus communis
KICEVPSTLFKGLCFSSNNCKHTCRKEQFTRGHCSVLTRACVCTKKC

SEQ ID NO: 63 - protein, 46 aa, Beta vulgaris subsp. vulgaris
AICKKPSKFFKGACGRDADCEKACDQENWPGGVCVPFLRCECQRSC

SEQ ID NO: 64 - protein, 47 aa, Miscanthus lutarioriparius
RVCRRRIAGFKGVCMSDHNCAQVCLQEGWGGGNCDGILRQCKCIRQC

SEQ ID NO: 65 - protein, 103 aa, Lotus japonicus
NTCSEALDQCDSMESCDQKCKGRHNGRRGSCNLGLCTCSYSCGPPGPKIPSNLCTAGLGLCSTKCGDSYCNSICASKYNQGVGFCYTSGPSINLCTCQYPCAL

SEQ ID NO: 66 - protein, 49 aa, Aquilegia formosa
RDCESQSHKFKGTCIRKSNCASVCQSEGFNGGHCRGVTRRCYCTAKCHE

SEQ ID NO: 67 - protein, 49 aa, Dichanthelium oligosanthes
RHCLSPSHHFKGLCMSSNNCANVCRTENFPGGECKTEGATRKCMCKKVC

SEQ ID NO: 68 - protein, 56 aa, Arabis nemorensis
CTTSIGPCEVGKNCSPKCNAMFGEIVTGFCDRSGGGIGICVCVTPCPPPPPKAPHM

SEQ ID NO: 69 - protein, 99 aa, Sesamum indicum
AVCQTRSQQFRGLCFRNDNCAAICEKEGYISGSCKGFLLRCICFKDCGATGGGPPDGSPDGDGGTPEGLTGSHVTSNISSVYKYGKNEAKPGNAEIKKG

SEQ ID NO: 70 - protein, 45 aa, Brassica rapa
RTCESPSTKFQGVCLNAKNCAGVCTGEGFTGGQCSSLKCYCTKTC

SEQ ID NO: 71 - protein, 49 aa, Tetracentron sinense
KTCTKRSRTWFGLCLNSNGCNRQCKKWEGAKHGACHAQFQGTACFCYYC

SEQ ID NO: 72 - protein, 71 aa, Microthlaspi erraticum
RETCDKPYSICWFWPFFSDSDAYCRESCLKVKDEKYEYYDGQCRPARSALPLPPECFCCVRERSRAEIESI

SEQ ID NO: 73 - protein, 62 aa, Vanilla planifolia
RRCRAQSFTFKGICYSSHSCALVCKKEGYETGHCSRREQHKIFCICAFKSLNQTKDTLQELD

SEQ ID NO: 74 - protein, 47 aa, Glycine max
KDCLTRRHGFQGRCLFDRQCAHVCRSDGFIGGQCRGPLRKCFCSRPC

SEQ ID NO: 75 - protein, 49 aa, Brachypodium distachyon
RHCLSQSHKFKGTCVRSGNCANVCKTENFPDGECKTQGLERKCFCKRVC

SEQ ID NO: 76 - protein, 49 aa, Brachypodium distachyon
KLCEERSQKFRGPCKFNTNCVAVCVTEGYTGGYCHKTVFHSHCMCTKEC

SEQ ID NO: 77 - protein, 47 aa, Brachypodium distachyon
KICRKRSAGYHGMCMSDRNCAQVCQAEGWGGGNCDGAVRRCKCSREC

SEQ ID NO: 78 - protein, 47 aa, Brachypodium distachyon
RECETESTKFEGLCIMHSHCPDVCVTEGFTGGKCSTWKRKCMCTKEC

SEQ ID NO: 79 - protein, 48 aa, Brachypodium distachyon
KKCSKPSGRFKGPCFFKVDCEQQCKKEGWPRGVCTGFAGRCVCKNYGC

SEQ ID NO: 80 - protein, 146 aa, Brachypodium distachyon
YVHGFCNSPSGSFRGPCFSDSHCKAACEHEYYKGDKSKPFTDGKCSDSGSQRLDGVGVGSRWSLLPTCICSIFCGSEKITWRPIITIRLRCQKTQNRRLRTTITIRLRRQKAQNRRRRTITIRLRRRNKSRQHKAASRRGKCWETS

SEQ ID NO: 81 - protein, 103 aa, Brachypodium distachyon
LHWFCNIPSGSFHGPCMSNDHCLAACKSEYYNGDKSKPFTGGQCSLSGSILPRCICSIFCGPPMAEHDDPETHRHHHPPLPPPSEGPELSPPHHHAPPPRHHH

SEQ ID NO: 82 - protein, 47 aa, Brachypodium distachyon
RDCEAPSHKFHGACVRDSNCASVCETEGFSGDKREGPLMRCYCTKQC

SEQ ID NO: 83 - protein, 81 aa, Brachypodium distachyon
GDELLCDVRLTKCEDVCYKSGKCSRCCKNHRFNHGHCDLKSHCYCYRGPNSGAGDGEQQRMLRVIPASPPPPAARHPPLHA

SEQ ID NO: 84 - protein, 49 aa, Setaria italica
TLCNLRSMGFVGVCWSNMSCANQCVLEGRTSGYCKGIPAIKYCMCTFEC

SEQ ID NO: 85 - protein, 47 aa, Setaria italica
DRCETHSRTYKGRCNNHNCWSICITEGNTGGFCKGTLLLKCMCTSEC

SEQ ID NO: 86 - protein, 48 aa, Setaria italica
DMCKKRSRTFEGRCGLNMNCATVCVAEHYTGGFCKGFFHRECMCTKDC

SEQ ID NO: 87 - protein, 50 aa, Setaria italica
RRCVSQSHKFVGSCMRKSNCQHVCQTEGFPWGECRFHGGLLRRCFCNKLC

SEQ ID NO: 88 - protein, 29 aa, Arabidopsis thaliana
GYCFDCARACMRRGKYIRTCSFERKLCRC

SEQ ID NO: 89 - protein, 18 aa, Oryza sativa Japonica
GECKSHGLERKCFCKKVC

SEQ ID NO: 90 - protein, 19 aa, Artemisia vulgaris
GACHKREAGKESCFCYFDC

SEQ ID NO: 91 - protein, 18 aa, Arabidopsis thaliana
GACHAQFPGFACFCYFNC

SEQ ID NO: 92 - protein, 18 aa, Arabidopsis thaliana
GYCNTLDDFSLCLCKYPC

SEQ ID NO: 93 - protein, 14 aa, Arachis duranensis
GTCMKMACWCAHNC

SEQ ID NO: 94 - protein, 16 aa, Malus baccata
GKCSLLTRTCMCTKKC

SEQ ID NO: 95 - protein, 16 aa, Oryza sativa
GRCSTFVRNCICIKPC

SEQ ID NO: 96 - protein, 14 aa, Triticum dicoccoides
GKCDHRRCVCTKGC

SEQ ID NO: 97 - protein, 16 aa, Pyrus ussuriensis x Pyrus communis
GHCSVLTRACVCTKKC

SEQ ID NO: 98 - protein, 15 aa, Beta vulgaris subsp. vulgaris
GVCVPFLRCECQRSC

SEQ ID NO: 99 - protein, 16 aa, Miscanthus lutarioriparius
GNCDGILRQCKCIRQC

SEQ ID NO: 100 - protein, 21 aa, Lotus japonicus
GFCYTSGPSINLCTCQYPCAL

SEQ ID NO: 101 - protein, 18 aa, Aquilegia formosa
GHCRGVTRRCYCTAKCHE

SEQ ID NO: 102 - protein, 18 aa, Dichanthelium oligosanthes
GECKTEGATRKCMCKKVC

SEQ ID NO: 103 - protein, 29 aa, Arabis nemorensis
GFCDRSGGGIGICVCVTPCPPPPPKAPHM

SEQ ID NO: 104 - protein, 16 aa, Sesamum indicum
GSCKGFLLRCICFKDC

SEQ ID NO: 105 - protein, 14 aa, Brassica rapa
GQCSSLKCYCTKTC

SEQ ID NO: 106 - protein, 17 aa, Tetracentron sinense
GACHAQFQGTACFCYYC

SEQ ID NO: 107 - protein, 18 aa, Microthlaspi erraticum
GQCRPARSALPLPPECFC

SEQ ID NO: 108 - protein, 15 aa, Vanilla planifolia
GHCSRREQHKIFCIC

SEQ ID NO: 109 - protein, 17 aa, Glycine max
GGQCRGPLRKCFCSRPC

SEQ ID NO: 110 - protein, 18 aa, Brachypodium distachyon
GECKTQGLERKCFCKRVC

SEQ ID NO: 111 - protein, 18 aa, Brachypodium distachyon
GYCHKTVFHSHCMCTKEC

SEQ ID NO: 112 - protein, 16 aa, Brachypodium distachyon
GNCDGAVRRCKCSREC

SEQ ID NO: 113 - protein, 16 aa, Brachypodium distachyon
GKCSTWKRKCMCTKEC

SEQ ID NO: 114 - protein, 17 aa, Brachypodium distachyon
GVCTGFAGRCVCKNYGC

SEQ ID NO: 115 - protein, 32 aa, Brachypodium distachyon
GKCSDSGSQRLDGVGVGSRWSLLPTCICSIFC

SEQ ID NO: 116 - protein, 19 aa, Brachypodium distachyon
GQCSLSGSILPRCICSIFC

SEQ ID NO: 117 - protein, 26 aa, Brachypodium distachyon
SVCETEGFSGDKREGPLMRCYCTKQC

SEQ ID NO: 118 - protein, 25 aa, Brachypodium distachyon
GKCSRCCKNHRFNHGHCDLKSHCYC

SEQ ID NO: 119 - protein, 18 aa, Setaria italica
GYCKGIPAIKYCMCTFEC

SEQ ID NO: 120 - protein, 17 aa, Setaria italica
GFCKGTLLLKCMCTSEC

SEQ ID NO: 121 - protein, 17 aa, Setaria italica
GFCKGFFHRECMCTKDC

SEQ ID NO: 122 - protein, 19 aa, Setaria italica
GECRFHGGLLRRCFCNKLC

SEQ ID NO: 123 - protein, 75 aa, Capsicum annuum
DICTTPPRRYIGVCWSDTSCRKVCVEMENFEDGYCSKIQRKCLCTWTCVLDNIPNDAGTIMLQDAKTMQLLEEKF

SEQ ID NO: 124 - protein, 75 aa, Capsicum annuum
YNICKTKSKYFEGLCWVDSSCRKVCIEKDKFEDGHCSKLLRNCLCTKICAFDNIPNDAGTILVQDAKSLEAQLLE

SEQ ID NO: 125 - protein, 75 aa, Capsicum annuum
QNNICKTTSKHFKGLCFADSKCRKVCIQEDKFEDGHCSKLQRKCLCTKNCVFDNIPNDVGTILVEDAKTLEAQLL

SEQ ID NO: 126 - protein, 75 aa, Capsicum annuum
QQTCKTESQKFKGLCITKSSCRKACLKEKFTDGHCSKLQRRCLCTKPCVFENNPNEVETKLVGKTKTLREAMLEE

SEQ ID NO: 127 - protein, 79 aa, Capsicum annuum
RQTCKTESQKFKGLCITKSSCRKACLKEKFTDGHCSKLQRRCLCTKPCVFENNPNEDETNLVGKTKTLREAMLEEQIME

SEQ ID NO: 128 - protein, 75 aa, Capsicum annuum
YNICKTKSKKFEGLCFLDSSCRKVCVEQENFEDGHCSKLRRLCLCTKLCVFDNIPNDVGTILVQDAKTLEAQLLE

SEQ ID NO: 129 - protein, 75 aa, Capsicum annuum
QNICKTKSKYFTGLCWTDSSCRKVCIEKDKFQDGHCSKLQRNCLCTKICVFDNIPNDAGTILVQDAKTNEAQLLE

SEQ ID NO: 130 - protein, 75 aa, Capsicum annuum; identical to SEQ ID NO: 124

SEQ ID NO: 131 - protein, 75 aa, Nicotiana tabacum
RECKTESNTFPGLCITKPPCRKACISEGFTDGHCSKILRRCLCTKPCVFDEKMIKTGAETLAEEAKTLAAALFEE

SEQ ID NO: 132 - protein, 75 aa, Nicotiana tabacum
RECKTESNTFPGLCITKPPCRKACISEGFTDGHCSKILRRCLCTKPCVFDEKMIKTGAETFAEEAKTLAAALLEE

SEQ ID NO: 133 - protein, 75 aa, Nicotiana tabacum
RECKAVSNTLSGLCITKPPCREACISEGFTDGKCSKILKRCICHKPCVFDEKMVKTGAETLAEEVKTLAAAFLEE

SEQ ID NO: 134 - protein, 75 aa, Nicotiana tabacum
RECKAESITFSGLCVSKPPCRKACICEGFTNGHCSKILRRCLCTTPSVFDEKMIKTGAETLGEEAKTLAATLLEE

SEQ ID NO: 135 - protein, 75 aa, Nicotiana tabacum
QSTCKAESNTFPGICITKPPCRKACLSEGFTDGKCSKILRRCICYKPCVFDEKMTKTGAETLAEEAKTLAAALLE

SEQ ID NO: 136 - protein, 75 aa, Nicotiana tabacum
RECKTESNTFPGICITKPPCRKACISEKFTDGHCSKLLRRCLCTKPCVFDEKMIKTGAETLVEEAKTLAAALLEE

SEQ ID NO: 137 - protein, 75 aa, Petunia hybrida
ATCKAECATWEGICINKAPCVKCCKEQPENFTDGHCSKILRRCLCTKPCATAEATATLANEVKTMGEALVEVDMM

SEQ ID NO: 138 - protein, 75 aa, Petunia hybrida
ATCKAECPTWDGICINKGPCVKCCKAQPEKFTDGHCSKVLRRCLCTKPCATEEATATLANEVKTMAEALVEEDMM

SEQ ID NO: 139 - protein, 75 aa, Petunia hybrida
ATCKAECPTWDSVCINKKPCVACCKKAKFSDGHCSKILRRCLCTKECVFEKTEATQTETFTKDVNTLAEALLEAD

SEQ ID NO: 140 - protein, 75 aa, Solanum lycopersicum
QQICKAPSQTFPGLCFMDSSCRKYCIKEKFTGGHCSKLQRKCLCTKPCVFDKISSEVKATLGEEAKTLSEVVLEE

SEQ ID NO: 141 - protein, 68 aa, Solanum lycopersicum
QPMCKSTSQTFKGLCFTDSSCRKSCLKEEFEGGHCSKLQRKCLCTKICVFDKISNEVRTNFGWGSKNS

SEQ ID NO: 142 - protein, 78 aa, Solanum lycopersicum
ASSTRANQQAKSSKDYVLPTHHVEKLVSKRSLKVDIIFKLQSKCLCTKICVFEKNPNEVKPTLGGEAKTLSETMLEDD

SEQ ID NO: 143 - protein, 62 aa, Solanum lycopersicum
QHMCKSTSQTFKGLCFTYSSCRKGCLKEKFEGGHYIVANSKGSAYALRFVYLTKFQMKLNQL

SEQ ID NO: 144 - protein, 75 aa, Solanum lycopersicum
QYMCKSTSQTFKGLCFTDSSCRKACVTEEFTGGHCSKLQRKCLCTKVCVLEKDSNEVKTTLVGEAKTLSETVLEE

SEQ ID NO: 145 - protein, 75 aa, Solanum lycopersicum
QQMCKSTSQTFKGLCFTDSSCRKACVTEEFTGGHCSKLQRKCLCTKVCVFEKDSNEVKTTLVGEAKTLSETVLEE

SEQ ID NO: 146 - protein, 75 aa, Solanum pennellii
RQICKAPSQTFPGLCFMDSSCRKYCIKEKFTGGHCSTLQRRCLCTKPCVFDIISNEVKATLGEEAKTLSEVVLEE

SEQ ID NO: 147 - protein, 75 aa, Solanum pennellii
QQMCKSTSQTFKGLCFIDSSCRKACVTEEFTGGHCSKLQRKCLCTKVCVFEKDSNEVKTTLVGEAKTLSETVLEE

SEQ ID NO: 148 - protein, 49 aa, Cajanus cajus
AVCSKPSTYFFGPCVRRSTCRRACSHENYPDGKCSRFLGKCICYKPCTT

SEQ ID NO: 149 - protein, 46 aa, Eucalyptus grandis
ATCRKPSMYFSGACFSDTNCQKACNREDWPNGKCLVGFKCECQRPC

SEQ ID NO: 150 - protein, 55 aa, Eucalyptus grandis
MNIVTKVDGAICKKPSKFFKGACGRDADCEKACDQENWPGGVCVPFLRCECQRSC

SEQ ID NO: 151 - protein, 49 aa, Eucalyptus grandis
ATCAKPSKYYKGGCFATISVSACKKTCSRENWPDGACVYPFRCECRRPC

SEQ ID NO: 152 - protein, 49 aa, Eucalyptus grandis
KTCAKPSKYYKGGCFSTISVSACKKTCSRENWPDGDCVFPYRCECRRPC

SEQ ID NO: 153 - protein, 49 aa, Eucalyptus grandis
ANCAKPSKFYRGPCFNSISVKPCTQRCSQENWPIGVCTKDLRCECQRPC

SEQ ID NO: 154 - protein, 49 aa, Eucalyptus grandis
FSVISDSACKKACNQENWPIGICVPVFRCECRRSCQAMFSPESSSIKYE

SEQ ID NO: 155 - protein, 49 aa, Brassica napus
KARMCETSSQLFNGPCLSTTNCANICQNEGFPDGDCKGFRLRCICNRPC

SEQ ID NO: 156 - protein, 47 aa, Brassica rapa
RTCESQSHRFKGPCVSENNCANVCHNEGFGGGKCRGLRRRCFCTRHC

SEQ ID NO: 157 - protein, 50 aa, Brassica napus
IEGRMCQSRSHHFRSICLSNHNCANICRGERFSGGRCHGLHRKCYCTRLC

SEQ ID NO: 158 - protein, 45 aa, Brassica rapa; identical to SEQ ID NO: 70

SEQ ID NO: 159 - protein, 47 aa, Brassica cretica
RTCESKSHRFKGTCVSSTNCGNVCHNEGFGGGKCRGFRRRCYCTRHC

SEQ ID NO: 160 - protein, 47 aa, Brassica napus
RTCESKSHKFRGPCVSRHNCANVCHNEGFHGGKCRGFRRRCYCTRHC

SEQ ID NO: 161 - protein, 48 aa, Zostera marina
RTCVSKSEKFKGVCLINTNCAHVCQTEGFPDGDCRGSVRRRCFCIRPC

SEQ ID NO: 162 - protein, 47 aa, Microthlaspi erraticum
RTCESKSHRFKGPCVSTHNCGNVCHNEGFGGGKCRGFRRRCYCTRHC

SEQ ID NO: 163 - protein, 47 aa, Arabidopsis lyrata
RTCASKSEKFKGSCVSNTNCANICHTEGFPEGACRGLRHHCFCTRPC

SEQ ID NO: 164 - protein, 53 aa, Thalspi arvense
QVRMCESKSHEFKGFCTKWRICRDVCITEGFTDGGCHGVLRHCVCKKPCLLSN

SEQ ID NO: 165 - protein, 54 aa, Actinidia chinensis
MIKQGEARVCESKSHGFKGACLIDHNCALVCRNEGFSGGKCRGFRHRCFCTKIC

SEQ ID NO: 166 - protein, 63 aa, Actinidia chinensis
KLCTAPSSTFKYLCISDQNCSSACEQEGFTSGECEGLRRRCICGKPCGMEVSKTKFDNGIGGE

SEQ ID NO: 167 - protein, 47 aa, Arabidopsis lyrata
RTCETSSNLFNGPCLSSSNCANVCHNEGFSDGDCRGFRRRCLCTRPC

SEQ ID NO: 168 - protein, 54 aa, Arabidopsis lyrata
QPQLCETKSLNYRGLCMKWRNCKRVCISEGFPDGRCKGFFNNKCVCRKPCALST

SEQ ID NO: 169 - protein, 47 aa, Arabidopsis lyrata
RTCASQSQRFKGKCVSDTNCANVCHNEGFPGGDCRGFRRRCFCTRNC

SEQ ID NO: 170 - protein, 47 aa, Arabidopsis lyrata
RTCESKSHRFKGPCVSTHNCANVCHNEGFGGGKCRGFRRRCYCTRHC

SEQ ID NO: 171 - protein, 45 aa, Arabidopsis lyrata
RTCESPSNKFQGVCLNSQSCAKACPSEGFSGGRCSSLRCYCSKAC

SEQ ID NO: 172 - protein, 59 aa, Arabidopsis lyrata
EVSPVDNKICKTRSDRFSGVCLSTNNCAIICQQFEHFDGGHCEFDGALRRCMCTKQCNN

SEQ ID NO: 173 - protein, 54 aa, Arabidopsis lyrata; identical to SEQ ID NO: 168

SEQ ID NO: 174 - protein, 52 aa, Arabidopsis lyrata
QQMCETQSINFRGMCTKWRNCQRVCMSEGFPDGRCKGFIRKCICTKPCPVST

SEQ ID NO: 175 - protein, 53 aa, Arabidopsis lyrata
IIGSDGRTCQSKSHHFKYMCTSSHNCAIVCRNEGFSGGRCHGFHRRCYCTRLC

SEQ ID NO: 176 - protein, 49 aa, Aquilegia oxysepala; identical to SEQ ID NO: 66

SEQ ID NO: 177 - protein, 49 aa, Arachis hypogaea
GGRHCESKSHRFKGMCVHERNCASVCNLEGFSGGKCRGFRRRCFCTRHC

SEQ ID NO: 178 - protein, 63 aa, Arachis hypogaea
EGRKCDSQSHHFKGKCFSDTNCASVCHGSFIHVFVELDLQSFEFTSCFSLEIKKFSSRSGQLF

SEQ ID NO: 179 - protein, 48 aa, Arachis hypogaea
RTCASQSHRFKGVCLSDTNCASVCKTEGFPSGDCHGFRRRCFCTKHCA

SEQ ID NO: 180 - protein, 55 aa, Arachis hypogaea
RTCASQSHRFKGVCLSDTNCASVCKTEGFPSGDCHGFRRRCFCTKHYRKIRSQDS

SEQ ID NO: 181 - protein, 49 aa, Arachis hypogaea
EGRHCESKSHRFKGMCVHERNCASVCNLEGFSGGKCRGFRRRCFCTRHC

SEQ ID NO: 182 - protein, 47 aa, Arachis hypogaea
RVCQSKSHMFKGGCWGDHNCALVCRNEGFSGGRCRGFRRRCFCTKLC

SEQ ID NO: 183 - protein, 47 aa, Arabidopsis thaliana; identical to SEQ ID NO: 167

SEQ ID NO: 184 - protein, 47 aa, Arabidopsis thaliana
RTCESQSHRFKGTCVSASNCANVCHNEGFVGGNCRGFRRRCFCTRHC

SEQ ID NO: 185 - protein, 47 aa, Arabidopsis thaliana
RTCASQSQRFKGKCVSDTNCENVCHNEGFPGGDCRGFRRRCFCTRNC

SEQ ID NO: 186 - protein, 47 aa, Arabidopsis thaliana; identical to SEQ ID NO: 170

SEQ ID NO: 187 - protein, 45 aa, Arabidopsis thaliana; identical to SEQ ID NO: 171

SEQ ID NO: 188 - protein, 57 aa, Arabidopsis thaliana
EVSPLDNKICKTRSDRFSGVCISTNNCAIICQQFEHFDGGHCEFDGAFRRCMCTKQC

SEQ ID NO: 189 - protein, 57 aa, Arabidopsis thaliana
QPQLCETKSLNYRGLCLKWRSCKRVCISEGFPDGRCKGFFNNKCVCRKPCALLSTEN

SEQ ID NO: 190 - protein, 53 aa, Arabidopsis thaliana
QQMCEAKSLDWKGMCLKWRNCRQVCISEGFTDGRCKGFTRKCICSKPCFVLPN

SEQ ID NO: 191 - protein, 47 aa, Arabidopsis thaliana
RTCQSKSHHFKYMCTSNHNCAIVCRNEGFSGGRCHGFHRRCYCTRLC

SEQ ID NO: 192 - protein, 58 aa, Acer truncatum
EMGQNQMVAEARTCESQSHKFKGTCVSKSNCAHVCQTEGFPGGHCRGFRRRCFCAKNC

SEQ ID NO: 193 - protein, 55 aa, Acer truncatum
QNEMVAEARTCESQSHKFKGTCLSKSNCANVCKTEGFPGGHCRGFRRRCFCTKKC

SEQ ID NO: 194 - protein, 49 aa, Brassica carinata
KARMCETSSQLFNGPCLSTTNCANICQNEGFPDGDCHGFRLRCICNRPC

SEQ ID NO: 195 - protein, 47 aa, Brassica carinata
RTCESQSHRFKGTCVSENNCANVCHNEGFGGGKCRGFRRRCFCTRNC

SEQ ID NO: 196 - protein, 75 aa, Brassica carinata
MDPVTVEARTCESKSHKFRGLCVSTHNCANVCHTKVKRSKECVSTDRTVPLLVPKKVPQAVVAVVSVAPAPNPAS

SEQ ID NO: 197 - protein, 47 aa, Brassica carinata; identical to SEQ ID NO: 160

SEQ ID NO: 198 - protein, 47 aa, Brassica carinata
RTCESQSHRFKGPCVSDNNCANVCHNEGFGGGKCRGLRRRCFCTTHC

SEQ ID NO: 199 - protein, 47 aa, Brassica carinata
RTCESKSHKFRGLCVSRHNCANVCHNEGFHGGKCRGFRRRCYCTRHC

SEQ ID NO: 200 - protein, 47 aa, Brassica carinata
RTCESKSHRFKGTCVSTTNCANVCHNEGFPGGKCRGFRRRCFCTRHC

SEQ ID NO: 201 - protein, 47 aa, Brassica carinata; identical to SEQ ID NO: 159

SEQ ID NO: 202 - protein, 49 aa, Brassica carinata; identical to SEQ ID NO: 155

SEQ ID NO: 203 - protein, 49 aa, Brassica oleracea; identical to SEQ ID NO: 155

SEQ ID NO: 204 - protein, 47 aa, Brassica oleracea
RTCESQSHRFKGPCVSDNNCANVCHNEGFGGGKCRGLRRRCFCTRHC

SEQ ID NO: 205 - protein, 50 aa, Brassica oleracea; identical to SEQ ID NO: 157

SEQ ID NO: 206 - protein, 72 aa, Brassica oleracea
QQMCEAKSLNFKGMCLKWKNCKHVFFTRKQLIKLSKKDVAAAEEEKILKETCMKLVQGQQMCEAKSINFKGM

SEQ ID NO: 207 - protein, 47 aa, Brassica oleracea; identical to SEQ ID NO: 159

SEQ ID NO: 208 - protein, 47 aa, Brassica oleracea; identical to SEQ ID NO: 199

SEQ ID NO: 209 - protein, 49 aa, Brassica rapa; identical to SEQ ID NO: 155

SEQ ID NO: 210 - protein, 47 aa, Brassica rapa; identical to SEQ ID NO: 156

SEQ ID NO: 211 - protein, 47 aa, Brassica rapa
RMCQSRSHHFRSICLSNHNCANICRGERFSGGRCHGLHRKCYCTRLC

SEQ ID NO: 212 - protein, 47 aa, Brassica rapa; identical to SEQ ID NO: 159

SEQ ID NO: 213 - protein, 47 aa, Brassica rapa; identical to SEQ ID NO: 160

SEQ ID NO: 214 - protein, 49 aa, Corchorus olitorius; identical to SEQ ID NO: 155

SEQ ID NO: 215 - protein, 47 aa, Corchorus olitorius; identical to SEQ ID NO: 204

SEQ ID NO: 216 - protein, 50 aa, Corchorus olitorius; identical to SEQ ID NO: 157

SEQ ID NO: 217 - protein, 72 aa, Corchorus olitorius
QQMCEAKSINFKGMSLKWKNCKHFIHENELIKLSRKEVAAAEEEKFLKETCMKLVQGQQMCEAKSINFKGIC

SEQ ID NO: 218 - protein, 47 aa, Corchorus olitorius; identical to SEQ ID NO: 159

SEQ ID NO: 219 - protein, 45 aa, Corchorus olitorius; identical to SEQ ID NO: 70

SEQ ID NO: 220 - protein, 47 aa, Corchorus olitorius; identical to SEQ ID NO: 199

SEQ ID NO: 221 - protein, 48 aa, Capsicum annuum
RSCESASQRFNGYCVRSSNCASICSTEGFTDGKCKGTIRRHCTCIRNC

SEQ ID NO: 222 - protein, 48 aa, Capsicum annuum
RVCISQSHGFKGPCGHDHNCALVCRNEGFSGGDCIGVFIRKCYCTKNC

SEQ ID NO: 223 - protein, 48 aa, Capsicum annuum
KICEALSGNFKGLCLSSRDCGNVCRREGFTDGSCIGFRLQCFCTKPCA

SEQ ID NO: 224 - protein, 47 aa, Capsicum annuum
RKCESQSHRFKGPCVRKSNCASVCQTEGFTGGHCRGFRRRCFCTKHC

SEQ ID NO: 225 - protein, 47 aa, Capsicum annuum
RHCESKSQRFKGPCVRQKNCAAVCETEGFPGGDCRGFRRRCFCTRPC

SEQ ID NO: 226 - protein, 47 aa, Capsicum annuum
RTCESQSHRFKGVCASETNCASVCQTEGFSGGDCRGFRRRCFCTRPC

SEQ ID NO: 227 - protein, 51 aa, Capsicum annuum
KTCDTLSENFKGICFSSTNCATTCQNEDEEYTGGHCRGLHRRCFCTTECEL

SEQ ID NO: 228 - protein, 49 aa, Capsicum annuum
KTCERLSGTFKGICFSSKNCASVCENEDEEFTGGHCSGLRRRCYCTADC

SEQ ID NO: 229 - protein, 47 aa, Cannabis sativa
RVCESKSHHFKGLCVIDHNCATICRTEGFSGGNCQGFRQRCFCTRHC

SEQ ID NO: 230 - protein, 47 aa, Cannabis sativa
RTCEAQSHHFKGPCVRKSNCANVCRTEGFPSGHCRGFRRRCFCTKHC

SEQ ID NO: 231 - protein, 47 aa, Cannabis sativa
KTCEARSQSHRGICVTNHTCDNVCKIERFSSGYCSGIDRRCLCTKHC

SEQ ID NO: 232 - protein, 47 aa, Cardamine hirsuta
RTCQSSSQLFSGPRLSTSNCANVCQNEGFTDGDCRGFRRRCFCNKQC

SEQ ID NO: 233 - protein, 54 aa, Cardamine hirsuta
QQLCETKSTNYRGLCLRWRNCKRVCMSEGFPDGRCRGIFDNKCVCRKPCALSTA

SEQ ID NO: 234 - protein, 47 aa, Cardamine hirsuta
RTCESQSHRFKGTCVSASNCGNVCHNEGFNGGNCRGFRRRCFCTRHC

SEQ ID NO: 235 - protein, 47 aa, Cardamine hirsuta
RTCESQSHRFKGTCTNEQNCRNICHNEGFFGGKCRGFRRRCFCTRNC

SEQ ID NO: 236 - protein, 47 aa, Cardamine hirsuta
RMCESKSQQFRGLCMSSTNCATVCHNEGFPGGECRGFRRRCFCTRPC

SEQ ID NO: 237 - protein, 100 aa, Cardamine hirsuta
KPRRCESQSNKFKGKCVSETNCGNVCRTEGFTGGDCRGFRRRCFCTRNCCARHRMKLSMRLISAALLLLMLFVATGMGPVMVDARTCESKSHRFKGPCVS

SEQ ID NO: 238 - protein, 47 aa, Cardamine hirsuta
RVCETRSQKFKGPCVSENNCANVCQTEGFPDGDCNGILRRCYCNSPC

SEQ ID NO: 239 - protein, 45 aa, Cardamine hirsuta
KTCETPSTKFQGVCLNSQNCAKVCPTEGASGGRCNSLRCVCSKTC

SEQ ID NO: 240 - protein, 47 aa, Cardamine hirsuta
RICQSKSHHYKYMCTSNHNCAMVCRNEGFSGGQCHGFHRRCYCTRLC

SEQ ID NO: 241 - protein, 47 aa, Capsella rubela
RTCESSSNLFSGPCLSESNCANVCNTEGFPDGDCRGFRRRCMCTRPC

SEQ ID NO: 242 - protein, 47 aa, Capsella rubela
RTCESKSHRFKGPCVSGNNCKNVCHNEGFPGGRCRGFRRRCYCTRHC

SEQ ID NO: 243 - protein, 45 aa, Capsella rubela
RTCESPSSKFSGVCLNSQTCAKACPSEGFTGGRCNSLRCYCSKPC

SEQ ID NO: 244 - protein, 57 aa, Capsella rubela
EMGLAADKVCKTRSDRFSGVCLSNNNCAIICQQFENFDSGHCEFDGAFRRCLCTKNC

SEQ ID NO: 245 - protein, 47 aa, Capsella rubela
RMCQSKSHHFRRMCWSDHNCAMICRSERFSGGHCHGFHRRCYCTRLC

SEQ ID NO: 246 - protein, 47 aa, Cicer arientinum
RVCASQSHAFKGPCVGDHNCALVCRNEGFSGGNCQGFRRRCFCTKLC

SEQ ID NO: 247 - protein, 47 aa, Citrus clementina
RTCESLSHRFKGTCVRGSNCAAVCQTEGFPGGQCSGLRRRCFCSKPC

SEQ ID NO: 248 - protein, 77 aa, Citrus clementina
HDTEMGPVLCGDTGNEKGPSRRKAAAVAAIERTCETPSKRFKGPCVKESNCAAVCQTEGFQGGKCRGLRRRCFCTRP

SEQ ID NO: 249 - protein, 47 aa, Citrus clementina
RICESQSHRFKGPCIRKDNCAAVCQTEGFHGGRCRGFRRRCFCTKHC

SEQ ID NO: 250 - protein, 47 aa, Ceratophyllum demensis
RMCESPSRHYKGLCFRSSNCASICHIEHFSHGHCHGFRRRCICSRPC

SEQ ID NO: 251 - protein, 47 aa, Ceratophyllum demensis
RMCESPSRHYKGLCFRSSNCASICHIEHFSHGYCHGFRRRCICSRPC

SEQ ID NO: 252 - protein, 47 aa, Carpinus fangiana
RVCESQSHKFHGPCLGDHNCALVCRNEGFSGGDCKGLRRRCFCTRLC

SEQ ID NO: 253 - protein, 50 aa, Citrullus lanatus
AEARICESQSHRFKGPCVSKSNCAAVCQTEGFHGGHCRGFRRRCFCTKRC

SEQ ID NO: 254 - protein, 47 aa, Citrullus lanatus
RVCQSQSHHFHGACFSHHNCAFVCRNEGFSGGKCRGARRRCFCSKLC

SEQ ID NO: 255 - protein, 47 aa, Carya illinoinensis
RVCESKSHGFQGMCTRDHNCALVCRNEGFSGGKCRGFRHRCFCTKLC

SEQ ID NO: 256 - protein, 47 aa, Carya illinoinensis
RVCESKSHKFQGPCVRDNNCGLVCKNEGFTGGKCRGFRRRCFCTKIC

SEQ ID NO: 257 - protein, 58 aa, Citrullus lanatus
EEMMGGGRAEGRVCKSQSHSFHGPCMRDHNCAVVCRTEGFSGGDCVGFRRRCFCTRLC

SEQ ID NO: 258 - protein, 47 aa, Corchorus olitorius
RVCESKSHWFKGPCWGDHNCALVCRNEGFSGGRCRGFRHRCFCTKLC

SEQ ID NO: 259 - protein, 47 aa, Corchorus olitorius
RLCQSQSHGFKGGCLSNHNCALVCRNEGFSGGQCRGLRHRCFCTRRC

SEQ ID NO: 260 - protein, 32 aa, Carica papaya
  misc_feature (32)..(32): Xaa can be any naturally occurring amino acid
RTCQSQSHGFHGACLSHHNCAVVCQKEGFSGX

SEQ ID NO: 261 - protein, 47 aa, Carica papaya
RTCESQSHRFKGACGSRSNCAAVCQTEGFHGGHCRGFRRRCFCTKHC

SEQ ID NO: 262 - protein, 50 aa, Cucumis sativus
VEGRICESKSHGFKGPCMTDHNCALVCRNEGFSGGKCRGVRHRCFCTKLC

SEQ ID NO: 263 - protein, 50 aa, Cucumis sativus; identical to SEQ ID NO: 262

SEQ ID NO: 264 - protein, 73 aa, Daucus carota
TDTCEVRSAYFVSLCLIDHHCEMVCKVEGFPTGKCLGLVPHCICLGTCKQPSPPSPPPANPPESPPPVPPPTP

SEQ ID NO: 265 - protein, 47 aa, Daucus carota
RTCESQSHKFKGTCLSETNCGNVCKNEGFSGGNCRGLRRRCFCTRHC

SEQ ID NO: 266 - protein, 47 aa, Daucus carota
RTCESQSHKFKGTCVSRSNCANVCKNEGFPGGHCRGFRRRCFCIKHC

SEQ ID NO: 267 - protein, 47 aa, Davidia involucrata
RVCEAQSHGFKGACMSNHNCALVCRNEGFSGGKCRGFRHRCFCTKQC

SEQ ID NO: 268 - protein, 47 aa, Durio zibethin
RVCESKSHRFKGPCLSHHNCGMVCKNEGFSGGKCRGVRRRCFCTRRC

SEQ ID NO: 269 - protein, 52 aa, Durio zibethin
ISVEGRTCESQSHRFKGACVSENNCAAVCQTEGFLGGNCRGFRRRCFCTKQC

SEQ ID NO: 270 - protein, 52 aa, Durio zibethin
IPLEGRTCESQSHRFKGMCVRESNCATVCQTEGFQGGHCRGFHRRCFCTKHC

SEQ ID NO: 271 - protein, 47 aa, Durio zibethin
RTCESQSHRFKGTCLRKSNCAAVCQTEGFHGGHCRGFRRRCFCTKHC

SEQ ID NO: 272 - protein, 50 aa, Durio zibethin
GEARVCESKSHKYKGACFHDHNCAYVCRNEGFSGGKCHGLHHHCFCTKIC

SEQ ID NO: 273 - protein, 47 aa, Durio zibethin
RVCESKSHKFKGPCMGDHNCAYVCRNEGFSGGKCRGLRHRCFCTKIC

SEQ ID NO: 274 - protein, 47 aa, Erigeron canadensis
RMCQSQSHMYHGACLRDHNCALVCRNEGFSGGRCRGFRRRCFCTRLC

SEQ ID NO: 275 - protein, 71 aa, Erigeron canadensis
NEMGGPMVVEGRTLAVVTVIGAGGVQRWWRWWQCNGDSGGDGGTRAMVAVTNSGGSSSNGGGGDDWWWWWQ

SEQ ID NO: 276 - protein, 48 aa, Eutrema salsugenium
RMCTNPSKTYKGPCVSDTNCESVCQSEGFPQGNCHGFRRRCICGKPCA

SEQ ID NO: 277 - protein, 47 aa, Eutrema salsugenium
RTCETASHKFKGPCLGDRNCANVCQTEGFPGGDCQGLRRRCFCTRPC

SEQ ID NO: 278 - protein, 48 aa, Eutrema salsugenium
RTCDAPSHSFKGICTRKRNCESVCNSEGFADGDCKGFRRRCICNRPCA

SEQ ID NO: 279 - protein, 49 aa, Eutrema salsugenium
RTCVTPSHRFRGICVSKRNCESACNSEGFPDGNCKGFRRRCICNRPCAK

SEQ ID NO: 280 - protein, 51 aa, Eutrema salsugenium
LFGRCKKVPSRTYKGPCARDANCRSVCISEGYNGGDCHGFRRRCMCTDKCS

SEQ ID NO: 281 - protein, 47 aa, Eucalyptus grandis
RTCESQSQRFKGACVSKTNCASVCQTEGFHGGHCRGFRRRCFCTKHC

SEQ ID NO: 282 - protein, 50 aa, Eucalyptus grandis
IEGRMCESQSHKFHGLCMSDRTCVVVCKEEDFDDGKCRGFRHRCFCRKPC

SEQ ID NO: 283 - protein, 47 aa, Eucalyptus grandis
RVCESQSHGFHGSCVSGHNCALVCRNEGFSGGRCRGFRRRCFCTKLC

SEQ ID NO: 284 - protein, 47 aa, Fragaraia x ananassa
RVCESQSHGFHGTCIRAHNCALVCRHEGFSGGRCRGFRRRCFCTRRC

SEQ ID NO: 285 - protein, 47 aa, Fragaraia x ananassa
RTCETLSHKFKGTCLRESNCASVCQTEGFTGGDCRGLRRRCFCTKHC

SEQ ID NO: 286 - protein, 47 aa, Fragaraia x ananassa
RVCESQSHGFYGTCIHAHNCALVCRHEGFSGGRCRGFRRRCFCTRRC

SEQ ID NO: 287 - protein, 47 aa, Fragaraia x ananassa
AICKVPSKTFKGMCFSQKNCIVRCHSEGYGWGECSHLRRRCMCYKPC

SEQ ID NO: 288 - protein, 47 aa, Fragaraia x ananassa
RVCEVPSKTFTGLCFRDTHCIVRCHSEGYGYGKCSHVLRRCRCLKAC

SEQ ID NO: 289 - protein, 47 aa, Fragaraia x ananassa; identical to SEQ ID NO: 287

SEQ ID NO: 290 - protein, 47 aa, Fragaraia x ananassa; identical to SEQ ID NO: 284

SEQ ID NO: 291 - protein, 53 aa, Fragaraia x ananassa
GRPDPKTCESPSAKFEGPCIMGSNCALTCQKEGHDGGFCRDFWLPKCFCTKPC

SEQ ID NO: 292 - protein, 47 aa, Fragaraia x ananassa
RTCESLSHKFQGTCLRESNCASVCQTEGFTGGDCRGLRRRCFCTKHC

SEQ ID NO: 293 - protein, 53 aa, Fragaraia x ananassa
GRPDPKTCEPPSAKFEEPCIMGSNCALTCQKEGHDGGFCHDFWLPKCFCTKPC

SEQ ID NO: 294 - protein, 75 aa, Fragaraia x ananassa
LGPKVMVAHEAITCETQSIKYEGMCSREKPCASLCQTKGYSRGECHEVITLRLLYRPGKTDPIGQSKRRTYPLTN

SEQ ID NO: 295 - protein, 47 aa, Fragaraia x ananassa; identical to SEQ ID NO: 288

SEQ ID NO: 296 - protein, 53 aa, Fragaraia x ananassa
GRPDPKTCESPSAKFEGPCIMGSNCALTCQKEGHNGGFCRDFWLPKCFCTKPC

SEQ ID NO: 297 - protein, 53 aa, Fragaraia x ananassa
GRPDPKTCESPSGKFEGPCIMGSNCALTCQKEGHDGGFCRDFWLPKCFCTKPC

SEQ ID NO: 298 - protein, 53 aa, Fragaraia vesca; identical to SEQ ID NO: 291

SEQ ID NO: 299 - protein, 58 aa, Fragaraia vesca
LGPKVMVAHEAITCETQSIKYEGMCSREKPCASLCQTKGYSRGECHGVYKICVCATQC

SEQ ID NO: 300 - protein, 47 aa, Fragaraia vesca; identical to SEQ ID NO: 287

SEQ ID NO: 301 - protein, 48 aa, Fragaraia vesca
SICEIPSKKFKGLCIRDRKCIVICHSEGYGYGKCSHIGRRCMCLKTCD

SEQ ID NO: 302 - protein, 54 aa, Fragaraia vesca
MVAADDARKCESLSHNFNGLCLSESNCASVCATEGFTGGDCRGLRRRCFCTKQC

SEQ ID NO: 303 - protein, 47 aa, Fragaraia vesca; identical to SEQ ID NO: 285

SEQ ID NO: 304 - protein, 47 aa, Fragaraia vesca
RTCESLSHKFKGTCLSETNCASVCKTEGFSGGDCRGLRRRCFCTKHC

SEQ ID NO: 305 - protein, 47 aa, Fragaraia vesca; identical to SEQ ID NO: 284

SEQ ID NO: 306 - protein, 45 aa, Glycine max
MHLRSHRFKGMCLSEHNCASVSHLEGFTGGKCWGFRRRCFCSKHC

SEQ ID NO: 307 - protein, 47 aa, Glycine max; identical to SEQ ID NO: 74

SEQ ID NO: 308 - protein, 47 aa, Glycine max
RVCESQSHGFHGLCNRDHNCALVCRNEGFSGGRCKGFRRRCFCTRIC

SEQ ID NO: 309 - protein, 47 aa, Glycine max
RHCESKSHRFKGMCLSKHNCASVCHLEGFTGGKCRGFRRRCFCTRHC

SEQ ID NO: 310 - protein, 63 aa, Glycine max
RYIYIIPYVAVKRAEGRDCSSKSHGFKGLCFSDTNCAHVCRTEGFTGGDCGGFRRRCFCTRIC

SEQ ID NO: 311 - protein, 69 aa, Glycine max
RTCESQSHRFKGPCLSDTNCGSVCRTEGFSGGHCRGFSRRCFCTKEYDNYYKSSDVIRCKDGSGKFTKA

SEQ ID NO: 312 - protein, 47 aa, Glycine max
RTCESQSHRFKGPCLSDTNCGSVCRTERFTGGHCRGFRRRCFCTKHC

SEQ ID NO: 313 - protein, 48 aa, Gossypium hirsutum
GPICKATSNVYKGLCLIDDSCDIVCKREGFHKGNCKGFLRKCVCTNPC

SEQ ID NO: 314 - protein, 48 aa, Gossypium hirsutum
KTCETKSGKFKGMCMSSTNCASVCKSEPGFDGGHCRGFRRQCVCTKPC

SEQ ID NO: 315 - protein, 48 aa, Gossypium hirsutum
TTCETQSGKFKGMCMSSTNCASVCKSEPGFDGGYCRGFRRQCFCTKPC

SEQ ID NO: 316 - protein, 48 aa, Gossypium hirsutum
RTCETKSSEYKGICLFDANCDSICKVEPSFDGGHCHGFFRRCYCTKPC

SEQ ID NO: 317 - protein, 48 aa, Gossypium hirsutum
RTCETKSSEYKGICLFDANCDSVCKVEPGFDGGHCHGFFRRCYCTKPS

SEQ ID NO: 318 - protein, 51 aa, Gossypium hirsutum
MEDRICQSRSTEYRGMCLFDVNCDNICKTEPGFTGGHCHSFFRSCYCTKPC

SEQ ID NO: 319 - protein, 48 aa, Gossypium hirsutum
RKCESPSHKFKGMCMNRDNCAIVCQTEGYEDGKCEGFRRRCICIKPCF

SEQ ID NO: 320 - protein, 55 aa, Gossypium hirsutum
GPTIVGGRKCDSPSKKFNGMCMNHDNCATVCQTESHEDGKCEGFYRRCICIKPCF

SEQ ID NO: 321 - protein, 52 aa, Gossypium hirsutum
GVVEGRICESKSHRFKGVCLSDHNCGLVCRNEGFLDGWCRGFRHRCFCTRNC

SEQ ID NO: 322 - protein, 47 aa, Gossypium hirsutum
RVCQSQSHKFHGACVSSHNCAMVCRDEGFSGGRCRGFRRRCFCTKLC

SEQ ID NO: 323 - protein, 50 aa, Gossypium hirsutum
DGDKICESPSNAFKGLCLRDDNCDIVCKTEGFPNGDCKGFLRKCVCTKPC

SEQ ID NO: 324 - protein, 48 aa, Gossypium hirsutum
KTCETQSGKFKGMCMSSTNCASVCKSEPGFDGGHCQGFRRQCVCTKPC

SEQ ID NO: 325 - protein, 48 aa, Gossypium hirsutum
KTCVAESGKFKGMCMSSTNCASVCKSEPGFDGGHCQGFRRRCLCTKPC

SEQ ID NO: 326 - protein, 48 aa, Gossypium hirsutum
RTCETKSSEYKGICLFDANCDSICKVEPGFDGGHCHGFFRRCYCTKPC

SEQ ID NO: 327 - protein, 48 aa, Gossypium hirsutum
KTCVAESGKFKGMCMSSTNCASVCKSEPDFDGGHCQGFRRRCLCTKPC

SEQ ID NO: 328 - protein, 48 aa, Gossypium hirsutum
RTCETKSSEYKGICLFDANCDSICKIESGFDGGHCHGFFRRCYCTKPC

SEQ ID NO: 329 - protein, 65 aa, Gossypium hirsutum
DEEHKEIEIGTMAEEEHKICESKSGKFKGMCFLDANCDSICKAEPGSTGGHCHGIFRICYCTKPC

SEQ ID NO: 330 - protein, 48 aa, Gossypium hirsutum
RKCESPSHKFKGMCMNRDNCATVCQTEGYEDGKCEGFRRRCICIKPCF

SEQ ID NO: 331 - protein, 52 aa, Gossypium hirsutum
GMVEGRICESKSHRFKGVCLSDHNCGLVCRNEGFLDGWCRGFRHRCFCTRNC

SEQ ID NO: 332 - protein, 47 aa, Gossypium hirsutum
RVCQSQSHKFHGACLSSHNCAMVCRNEGFSGGRCRGFRRRCFCTKLC

SEQ ID NO: 333 - protein, 50 aa, Gossypium raimondii
DGDKICESRSNAFKGLCLRDNNCDIVCKTEGFPNGGCKGFIRKCVCTKPC

SEQ ID NO: 334 - protein, 48 aa, Gossypium raimondii
GTTCKATSNIYKGLCLLDDSCDIVCKREGFHSGNCKGFLRKCVCTNPC

SEQ ID NO: 335 - protein, 50 aa, Gossypium raimondii; identical to SEQ ID NO: 323

SEQ ID NO: 336 - protein, 48 aa, Gossypium raimondii
KTCEVESGKFKGMCMSSTNCASVCKSEPGFDGGHCQGFRRRCLCTKPC

SEQ ID NO: 337 - protein, 48 aa, Gossypium raimondii; identical to SEQ ID NO: 336

SEQ ID NO: 338 - protein, 48 aa, Gossypium raimondii
RTCETKSSEYKGICLFDANCDSICKIEPGFDGGHCHGFFRRCYCTKPC

SEQ ID NO: 339 - protein, 48 aa, Gossypium raimondii; identical to SEQ ID NO: 330

SEQ ID NO: 340 - protein, 47 aa, Vitis vinifera
RVCESQSHKFEGACMGDHNCALVCRNEGFSGGKCKGLRRRCFCTKLC

SEQ ID NO: 341 - protein, 47 aa, Vitis vinifera
RTCESQSHRFKGTCVRQSNCAAVCQTEGFHGGNCRGFRRRCFCTKHC

SEQ ID NO: 342 - protein, 47 aa, Helianthus annuus
RICESPSHKFKGICLSDTNCSSVCQSEGFPWGKCIGFRRRCFCLREC

SEQ ID NO: 343 - protein, 47 aa, Helianthus annuus
RTCESQSHKFKGTCLSDTNCANVCHSERFSGGKCRGFRRRCFCTTHC

SEQ ID NO: 344 - protein, 47 aa, Helianthus annuus
RMCQSQSHMFHGTCMRDHNCALVCRNEGFSGGKCRGFRRRCFCTRLC

SEQ ID NO: 345 - protein, 47 aa, Helianthus annuus
RICESQSQEFHGSCMSNHNCGLVCRTEGFSAGICRGVRGRCFCLKTC

SEQ ID NO: 346 - protein, 47 aa, Hydrangea macrophylla
RTCESKSNRFKGPCARDSNCATVCQTEGFYGGNCKGFRRRCFCTKNC

SEQ ID NO: 347 - protein, 47 aa, Hydrangea macrophylla
RTCESKSHRFKGPCVSATNCGSVCQTEGFHGGNCRGFRRRCFCTKHC

SEQ ID NO: 348 - protein, 71 aa, Vanilla planifolia
RRCRAQSFTFKGICYSSHSCALVCKKEGYETGHCSRGFCTLLKPANAEQRAQSLQVFASVPTPALLCVSKK

SEQ ID NO: 349 - protein, 58 aa, Cicer arietinum
SEVTIRVAEGRECRDLSHKFKGFCVRDRNCANVCSTEGFTNGHCDGFRRRCFCSRPCA

SEQ ID NO: 350 - protein, 49 aa, Beta vulgaris subsp. vulgaris
RVCETPSHKFQGICISSRNCASTCASEHFPGGYCRGLRRRCMCTRPCVN

SEQ ID NO: 351 - protein, 47 aa, Beta vulgaris subsp. vulgaris
RTCETPSRKFRGICVSRTNCANVCKTEGFPGGRCRGFRRRCFCLEHC

SEQ ID NO: 352 - protein, 47 aa, Beta vulgaris subsp. vulgaris
RICESPSYSFKGICVSRTNCANVCKTEGFPGGRCRGLRRRCFCLKHC

SEQ ID NO: 353 - protein, 47 aa, Spinacea oleracea
RTCESRSYRFKGICVSRTNCANVCKTEGFPGGRCRGVRRRCFCLKHC

SEQ ID NO: 354 - protein, 47 aa, Beta vulgaris subsp. vulgaris
RTCETPSHKFKGICVSRTNCANVCKTEGFPAGRCRGFRRRCFCLTHC

SEQ ID NO: 355 - protein, 47 aa, Ipomoea triloba
RICESKSHHYKGMCLRDHNCAMVCRTEGFSGGHCIGARRRCFCTRGC

SEQ ID NO: 356 - protein, 47 aa, Lupinus albus
RTCESQSHRFKGACLSDTNCGSVCHNEGFNGGHCRGFRRRCFCTKHC

SEQ ID NO: 357 - protein, 52 aa, Lupinus albus
FLGSHRCGKVLSSRFKGKCLTDHNCDSVCQLEGYVGGDCHGVRRRCFCNKKC

SEQ ID NO: 358 - protein, 47 aa, Lupinus albus
RHCESKSHRFKGLCHSDHNCASICLVEGFTSGKCHGFRQRCFCTKVC

SEQ ID NO: 359 - protein, 49 aa, Lupinus albus
RTRVCESQSHLFKGPCSRDHNCALVCRNEAFSGGRCKGFRRRCFCTKLC

SEQ ID NO: 360 - protein, 59 aa, Lupinus albus
MVIETEGVLGRNRCDPVLSSRFKGKCFSDHNCNSVCQLEGYTDGNCHGLRQRCFCNRKC

SEQ ID NO: 361 - protein, 59 aa, Lupinus albus
SDNNVGLKRAEGRVCQSKSHRFKGPCLSYSNCGHVCRNEGFSGGRCRGLRRRCFCTKNC

SEQ ID NO: 362 - protein, 59 aa, Lupinus albus
EMGPTMMEEARTCKSKSQRYKGSCYSDNNCAIICEGEGFTGGKCHGFFCKCWCKRPCLD

SEQ ID NO: 363 - protein, 69 aa, Lupinus albus
KECSTKSDRFRGTCFRSSNCAAVCNSENFPSGTCRGFPGTCYCTSNCAINAPITNPPPEVVEPPPEVVE

SEQ ID NO: 364 - protein, 48 aa, Lupinus albus
KTCESRSHKLKGFCYSSKRCAHVCTSHHNFIGGHCRGFIRRCHCTKPC

SEQ ID NO: 365 - protein, 48 aa, Lupinus albus
NNCKYESNKFHGVCLSDTNCESVCQTEGFPFGKCDGVLPRTCVCKKPC

SEQ ID NO: 366 - protein, 47 aa, Lotus japonica
RHCESKSHRFHGLCLSHHNCASVCHLEGFTGGKCRGFRKRCFCKKRC

SEQ ID NO: 367 - protein, 48 aa, Lotus japonica
RVCQSQSHRFRGGCWGDHNCALVCRNEGFSGGRCRGFRRRCFCTRLCN

SEQ ID NO: 368 - protein, 47 aa, Lotus japonica
RVCETKSHTFHHSCVSGHNCAQVCKTEGFTGGKCRGFRRRCFCTKRC

SEQ ID NO: 369 - protein, 47 aa, Lotus japonica
RDCESQSHRFKGPCVSDTNCASVCHGEKFSGGHCRGFRRRCFCTKHC

SEQ ID NO: 370 - protein, 55 aa, Lactuca sativa
MGVETKSCRYSSKTYKGLCFYSRNCDIICKAEGAPGGGHCQILTLRCFCNVCDPD

SEQ ID NO: 371 - protein, 47 aa, Manihot esculenta
RICESQSHRFKGVCVRKSNCAAICQTEGFHGGHCRGLRRRCFCTKHC

SEQ ID NO: 372 - protein, 47 aa, Manihot esculenta
RVCESKSHHFRGLCTIAHNCAMVCRNEGFSGGKCRGFRRRCFCTRLC

SEQ ID NO: 373 - protein, 47 aa, Manihot esculenta
RVCESKSHHFRGVCTIAHNCAIVCRNEGFSGGKCKGFRRRCFCTRLC

SEQ ID NO: 374 - protein, 47 aa, Manihot esculenta
RKCESQSHKFKGICLSDTNCASVCKTEGFTGGDCKGARRRCFCTRQC

SEQ ID NO: 375 - protein, 47 aa, Manihot esculenta
RKCESQSHRFKGMCTRHSNCAAICQTEGFHAGHCRGLRRRCFCTKHC

SEQ ID NO: 376 - protein, 47 aa, Malus domestica
RVCQSQSHGFHGTCLRHHNCALVCRNEGFSGGRCRGLRRRCFCTRLC

SEQ ID NO: 377 - protein, 57 aa, Malus domestica
RTESSKAVEGKICEFPSTLFKGLCFSSNNCKHTCRKEQFTRGKCSLLTRTCMCTKKC

SEQ ID NO: 378 - protein, 47 aa, Malus domestica
RTCESQSNRFKGTCVSKSNCAAVCQTEGFPGGNCRGLRRRCFCTKHC

SEQ ID NO: 379 - protein, 47 aa, Malus domestica
RTCESHSRKYKGTCLGKSNCASVCQTEGFHGGHCRGFRRRCFCTKHC

SEQ ID NO: 380 - protein, 63 aa, Malus domestica
RVCEVPSKTFRGLCFSDTHCIVKCHSEGFGYGKCSHVLRQCRCLKACGRLAESETTHGTETSP

SEQ ID NO: 381 - protein, 57 aa, Malus domestica
RTEPSKAVEGKICEVPSTLFKGLCISSNNCKHTCRKEQFTRGHCSVFTRACVCTKKC

SEQ ID NO: 382 - protein, 57 aa, Malus domestica
RVESSKAVEGKICEVPCTLFKGLCFSSNNCKHTCRKEQFTRGHCSVFTRACVCTKKC

SEQ ID NO: 383 - protein, 57 aa, Malus domestica
RTESNKAVEGKICEYPSTLFKGLCFSSNNCKHTCRKEQFTRGHCSVFTRACVCTKKC

SEQ ID NO: 384 - protein, 60 aa, Malus domestica
KTNENSSKNEKSSKICESPSHKYRGICFRSSNCATTCKKEGFMGGKCAGFRMRCVCTKKC

SEQ ID NO: 385 - protein, 60 aa, Malus domestica
KTKEESSKNEKSSKICESPSHKYRGICFRSSNCATTCKKEGFMGGKCAGFRMRCVCTKKC

SEQ ID NO: 386 - protein, 59 aa, Malus domestica
KTESSKAVEGKICEVPSTLFKGLCISSNNCKHTCRKEQFTRGKCIGDGFTRKCVCTKKC

SEQ ID NO: 387 - protein, 57 aa, Malus domestica
RSKSSKEVQERTCEAASGKFKGLCFSSTNCKNTCKREQFTGGKCRGFRRRCMCNKKC

SEQ ID NO: 388 - protein, 57 aa, Malus domestica
RSKSDKVAKERTCEAASGKFKGLCFSSTNCKNTCKVEKFTGGQCQGFRRRCMCNKKC

SEQ ID NO: 389 - protein, 57 aa, Malus domestica
RSKSGKAVKERTCEATSGKFKGMCFSSTNCKNTCKTEKFIGGECHGFRRRCICNKKC

SEQ ID NO: 390 - protein, 57 aa, Malus domestica
RTESSKAIEGKICEVPSTLFKGLCFSSNNCKHTCRKEQFTRGKCSGLTRTCVCTKKC

SEQ ID NO: 391 - protein, 57 aa, Malus domestica
RTESSKAVEGKICEVPSTLFKGLCFSSNNCKHTCRKEQFTRGKCSGLTRTCMCTKKC

SEQ ID NO: 392 - protein, 57 aa, Malus domestica
MTESNKAVEGKICEVPSTLFKGLCFSSNNCKHTCRKEQFTRGKCSGLTRTCMCTKKC

SEQ ID NO: 393 - protein, 60 aa, Malus domestica
VEARSKSSKVVEERTCEAASGKFKGLCFSSNNCKHTCKREQFTGGKCRGFRRRCMCNKKC

SEQ ID NO: 394 - protein, 47 aa, Malus domestica
RTCDSQSHRFKGSCVSKSNCATVCQTEGFRGGHCRGFRRRCFCTKHC

SEQ ID NO: 395 - protein, 60 aa, Malus domestica
KTKEKSSKNEKSSKTCESPSHKYHGICFRSSNCATTCKKEGFMGGKCTGFWMRCVCTKKC

SEQ ID NO: 396 - protein, 60 aa, Malus domestica
KTKEKSSKNEKSSKICESPSHKFHGICFRSSNCATTCKKEGFMGGKCTGFRMRCVCTKKC

SEQ ID NO: 397 - protein, 61 aa, Malus domestica
KIRHMSKDENSLTCESRSHKYHGPCFRFIFSSNCAVTCKGEGFTGGKCKGFHMRCICTKQC

SEQ ID NO: 398 - protein, 59 aa, Malus domestica
KTKGKPSKNVKLSKTCESPSQKYPGICRSSYCAAICKQQGLMGGKCTGFRRKCVCTKKC

SEQ ID NO: 399 - protein, 57 aa, Malus domestica; identical to SEQ ID NO: 59

SEQ ID NO: 400 - protein, 60 aa, Malus domestica
RSGMTSKGAAEKKKCDSHPSSKFKGVCFFTNNCVDTCKLEGSSGGQCRGFRRRCICTKQC

SEQ ID NO: 401 - protein, 57 aa, Malus domestica
KSKSSKEVEKRTCEAASGKFKGMCFSSNNCANTCAREKFDGGKCKGFRRRCMCTKKC

SEQ ID NO: 402 - protein, 47 aa, Malus domestica
RTCESQSNRFKGSCVSKSNCAAVCQTEGFPGGNCRGLRRRCFCTKHC

SEQ ID NO: 403 - protein, 47 aa, Malus domestica
RTCESQSRKFKGTCLSKTNCASVCQTEGFPGGNCRGFRRRCFCTKHC

SEQ ID NO: 404 - protein, 57 aa, Medicago truncatula
QPTHVEEPEARTCDSQSHSFKGVCWIKHNCANVCKTEGFTGGHCHGFRRRCFCSKPC

SEQ ID NO: 405 - protein, 76 aa, Medicago truncatula
EEFNFAPFTPLHFRVESCFTRSRRYNGICLSDESCFKACSLEGYACGRCEKRRPYLGDHCCCKKSCQLKLVLAVLC

SEQ ID NO: 406 - protein, 50 aa, Medicago truncatula
RKHCREKSRLFEELCFNSEDCANTCRYEGFHLGGKCWGLFRTCYCKKKCR

SEQ ID NO: 407 - protein, 47 aa, Medicago truncatula
RHCESKSHRFKGMCMSDHNCASVCHVEGFPGGNCRGFRRRCFCKKRC

SEQ ID NO: 408 - protein, 47 aa, Medicago truncatula
RTCESQSHKFKGPCASDHNCASVCQTERFSGGHCRGFRRRCFCTTHC

SEQ ID NO: 409 - protein, 47 aa, Medicago truncatula
RTCETPSNSFKGACFSDTNCASVCQTEGFPGGHCKGFRQRCFCTKPC

SEQ ID NO: 410 - protein, 47 aa, Medicago truncatula
RTCETPSNNFKGLCVSDTNCASVCQTEGFPGGHCEGFRQRCFCTKPC

SEQ ID NO: 411 - protein, 50 aa, Medicago truncatula
RKCLSQSHSFKGLCLSDQNCATVCLTEGFTDGRCRGFRQRCFCSKPCLKV

SEQ ID NO: 412 - protein, 47 aa, Medicago truncatula
RRCESKSHKFKGPCSRDSNCASVCRGEGFTGGDCRGFRRRCFCTRNC

SEQ ID NO: 413 - protein, 47 aa, Medicago truncatula
RRCESQSHKFKGPCVSDSNCGSVCRGEGFIGGDCRGVRHRCFCTRNC

SEQ ID NO: 414 - protein, 48 aa, Cucumis melo
RVCESPSHNFKGLCFSDTNCGNICKTEGFSGGLCRGFRRRCFCTKHCV

SEQ ID NO: 415 - protein, 47 aa, Cucumis melo
RMCQSQSHHFHGTCIRDHNCALVCRTEGFSGGECVGFRRRCFCTHRC

SEQ ID NO: 416 - protein, 58 aa, Mimulus guttatus
VKEVGTVEDEKKVCKYKSKKFWGVCLSDIRCTDVCKREGFQGGDCQGIRRRCLCYNRC

SEQ ID NO: 417 - protein, 48 aa, Mimulus guttatus
TRTCEAKSHRFKGICARKANCAAVCQTEGFHGGHCRGFRRRCFCTKHC

SEQ ID NO: 418 - protein, 47 aa, Mimulus guttatus
RVCESKSASYKGVCVLDVTCKLACLNEGFADGDCEGVRRRCMCRRPC

SEQ ID NO: 419 - protein, 39 aa, Mimulus guttatus
FKGICVLDETCRLACLNEGFSDGDCEGLRRRCLCRKPCK

SEQ ID NO: 420 - protein, 72 aa, Mimulus guttatus
ALCETKSSIFTGACFSDRNCETICEKEGFLNGRCKLWKCICSKDCGIGGGGGVVVPPGGGGDVPPGGGDDVP

SEQ ID NO: 421 - protein, 48 aa, Mimulus guttatus
RMCETQSHGFKGQCWSEHNCRLVCAHEGFRGGKCRGFRKRCFCTRPCH

SEQ ID NO: 422 - protein, 47 aa, Mimulus guttatus
RTCLSQSQRFRGPCVSDTNCRLVCHNEGFSGGDCQGFRRRCFCTKHC

SEQ ID NO: 423 - protein, 64 aa, Mimulus guttatus
QSNVDGEIADYLKIEEKKRCNYESKKFWGICLSDSKCGEVCTKEGFEGGDCKGLRRRCYCFNNC

SEQ ID NO: 424 - protein, 71 aa, Mimulus guttatus
SLCETPSRYFNGICFSRRNCGAICEKEGFSSGRCKAFKCVCGKDCNAAGGGGGGGGGDPGLGPPVEGPPDQ

SEQ ID NO: 425 - protein, 47 aa, Mimulus guttatus
RTCESQSHGFKGRCLSDHNCGMVCKNEGFSNGWCRGFRQRCFCTRAC

SEQ ID NO: 426 - protein, 47 aa, Mimulus guttatus
RTCTSKSSNFKGLCFSSHNCKAVCNGENFPDGKCHGFKRRCFCQKPC

SEQ ID NO: 427 - protein, 47 aa, Mimulus guttatus
RTCESQSFNFRGPCVIDHNCKAVCESEGFTDGDCQGFRRRCFCRKPC

SEQ ID NO: 428 - protein, 62 aa, Handroanthus impetiginosus
ALCEKPSRLYNGSCTEERLCIEICQKEAYPSGRCDLLKCMCINKCPPVGAGQGAGSDGQRGI

SEQ ID NO: 429 - protein, 47 aa, Nicotiana tabacum
RTCESQSHRFKGPCSRDSNCATVCLTEGFSGGDCRGFRRRCFCTRPC

SEQ ID NO: 430 - protein, 47 aa, Nicotiana tabacum
RHCESQSQRFRGLCVREKNCAAVCETEGFSGGDCRGLRRRCFCTRPC

SEQ ID NO: 431 - protein, 47 aa, Nicotiana tabacum
RVCMSPSHGYHGPCWHDHNCALVCRNEGFSGGDCVGIQRKCYCTKLC

SEQ ID NO: 432 - protein, 47 aa, Nicotiana tabacum
RTCESQSHRFHGTCVRGSNCASVCQTEGFIGGNCRGLRRRCFCTRNC

SEQ ID NO: 433 - protein, 58 aa, Nicotiana tabacum
TEMGPMTIAEARRCESKSQRFRGPCVREKNCAAVCETEGFSGGDCRGLRRRCFCTRPC

SEQ ID NO: 434 - protein, 48 aa, Nelumbo nucifera
RTCESQSHRFKGICVRKSNCAAVCQTEGFHGGHCRGFRRRCFCTKHCA

SEQ ID NO: 435 - protein, 13 aa, Nelumbo nucifera
CRDGAKSSRGKDL

SEQ ID NO: 436 - protein, 49 aa, Nelumbo nucifera
KMCKTPSRQFRGLCLIGRNCDNVCRGEGFPDGDCDGFRRRCICSRPCRP

SEQ ID NO: 437 - protein, 100 aa, Nelumbo nucifera
RMCDTPSQKFVGLCLIDRNCENVCKTEGFPAGHCEGLRRRCFCSEPCPPNNGMERKFLCGVLLFLTLLYASLGEEMVVNEGPADGGRVCSTPSLNYRGPC

SEQ ID NO: 438 - protein, 49 aa, Nelumbo nucifera
RMCDTPSRLFRGLCLINRNCDNTCRVEGFPDGHCKGLRRRCFCSRPCRP

SEQ ID NO: 439 - protein, 49 aa, Olea europea
RICESLSHRFKGPCVRNANCAAVCQTEGFPGGHCRGFRRRCFCTKPCSD

SEQ ID NO: 440 - protein, 47 aa, Olea europea
RICESLSHRFKGPCVRRGNCAAVCQTEGFPGGLCRGFRRRCFCTKHC

SEQ ID NO: 441 - protein, 70 aa, Oryza sativa
IICKARSKMYRGKCRGNRNCAMICVHEEYTGGYCSKGVFSKCMCTKRCGGGGGGGGGGGGGGGDEPPLRE

SEQ ID NO: 442 - protein, 70 aa, Oryza sativa
ICHDHSQTFKGMCFHTSNCIACCTNEGYTGGYCKPFTYRCMCTKDCGGDSPPDDPPPAMPTSPAATTTVA

SEQ ID NO: 443 - protein, 59 aa, Oryza sativa
RLCEKPSSHFKGLCLRSQNCDNECMLEGYMDGKCKYLTRRCICSMECATMSNVGLTPTK

SEQ ID NO: 444 - protein, 49 aa, Oryza sativa
RHCLSQSHRFKGMCVSSNNCANVCRTESFPDGECKSHGLERKCFCKKVC

SEQ ID NO: 445 - protein, 48 aa, Oryza sativa
RTCESQSHRFKGPCARKANCASVCNTEGFPDGYCHGVRRRCMCTKPCP

SEQ ID NO: 446 - protein, 49 aa, Oryza sativa
RHCVSQSHRFVGACMRKSNCEHVCMTEGFPWGECRFHGIERKCFCKKRC

SEQ ID NO: 447 - protein, 47 aa, Petunia axillaris
RTCESQSHRFKGPCVSKSNCASVCQTEGFHGGHCRGLRRRCFCTKHC

SEQ ID NO: 448 - protein, 49 aa, Petunia axillaris
RYRVCLSPSHRYKGMCLRDHNCALVCRNEGFSGGDCIGFRRRCFCTKLC

SEQ ID NO: 449 - protein, 49 aa, Petunia axillaris
EARICESQSHRFHGTCVRESNCASVCQTEGFIGGNCRGFRRRCFCTRNC

SEQ ID NO: 450 - protein, 47 aa, Petunia axillaris
RTCETPSQRFKGPCVSDTNCANVCQTEGFPGGDCQGLRRRCFCTKPC

SEQ ID NO: 451 - protein, 47 aa, Petunia axillaris
RTCETLSHRFRGPCVREKNCAAVCETEGFSGGDCRGLRRRCFCTRPC

SEQ ID NO: 452 - protein, 47 aa, Petunia axillaris
RVCMSPSHGYRGPCIRDHNCALVCRDEGFSGGDCVGIQRKCYCTKLC

SEQ ID NO: 453 - protein, 50 aa, Petunia axillaris
RTCEHRSQHYYGACFANNSNMNNNCDSMCKCDGFDGGFCRLTNCFCHVKC

SEQ ID NO: 454 - protein, 47 aa, Petunia axillaris
KLCQYKSKTFHGVCVTGNTCNQKCQSEAFDGGRCQGVRHRCYCYRTC

SEQ ID NO: 455 - protein, 51 aa, Petunia axillaris
RVCRTNSKRYSGPCFNNNNNRNQNCDFICRRCEGFEGGHCSSRTCVCTKSC

SEQ ID NO: 456 - protein, 47 aa, Punica granatum
RTCESQSHKFRGTCVSNTNCGSVCQTEGFHGGHCRGFRRRCFCTKQC

SEQ ID NO: 457 - protein, 47 aa, Punica granatum
RICESKSHRFRGVCMSNSNCASVCHNEGFPGGRCRGVRKRCFCTKVC

SEQ ID NO: 458 - protein, 47 aa, Solanum tuberosum
RTCESQSHRFKGPCVSEKNCASVCETEGFSGGDCRGLRRRCFCTRPC

SEQ ID NO: 459 - protein, 47 aa, Solanum tuberosum
RNCESLSHRFKGPCTRDSNCASVCETERFSGGNCHGFRRRCFCTKPC

SEQ ID NO: 460 - protein, 47 aa, Solanum tuberosum
RTCESQSRRFKGLCFSKSNCGSVCHTEGFSGGHCRGFRRRCFCTRHC

SEQ ID NO: 461 - protein, 47 aa, Solanum tuberosum
RTCESQSHKYKGPCVRKSNCGAVCQTEGFTGGHCRGVRRRCFCTKHC

SEQ ID NO: 462 - protein, 47 aa, Solanum tuberosum
RTCESQSHSFKGPCVGDTNCASVCQTEGFIGGDCRGLRRQCFCTRNC

SEQ ID NO: 463 - protein, 47 aa, Solanum tuberosum
RTCESQSNRFKGTCVRDSNCATVCQTEGFIGGNCRGFRRRCFCTRNC

SEQ ID NO: 464 - protein, 49 aa, Solanum tuberosum
RVCISQSHRYKGPCLRDHNCALVCRNEYFSGGDCIGFGFHRKCYCTKFC

SEQ ID NO: 465 - protein, 46 aa, Phaseolus vulgaris
KTCFTKKEGWGRCIADFTCARSCRNVGYIGGKCQGLTRRCFCVLNC

SEQ ID NO: 466 - protein, 50 aa, Phaseolus vulgaris
AEGKICRTKSRKYTGACYSNDICANYCKGDGFDDGDCGGLSHICFCTKNC

SEQ ID NO: 467 - protein, 47 aa, Phaseolus vulgaris
RLCESPSHSFKGLCFNDRNCANVCLTEGFTGGKCEGARHRCFCTIIC

SEQ ID NO: 468 - protein, 47 aa, Phaseolus vulgaris
RLCWSESQAFKGLCFSNDNCATVCLTEGFTGGKCEGLRRRCFCSKNC

SEQ ID NO: 469 - protein, 47 aa, Phaseolus vulgaris
RGCESRSRHLRGKCESDENCASACRTERFSGGHCRGFRCRCFCDKRS

SEQ ID NO: 470 - protein, 47 aa, Phaseolus vulgaris
RTCESQSHRFKGPCVSDTNCASVCRTERFSGGHCRGFRRRCFCTKHC

SEQ ID NO: 471 - protein, 47 aa, Phaseolus vulgaris
RVCESQSHGFKGACTGDHNCALVCRNEGFSGGNCRGFRRRCFCTKIC

SEQ ID NO: 472 - protein, 47 aa, Phaseolus vulgaris
RHCESKSHRFKGMCLSDRNCASVCHLEGFTGGNCRGFRRRCFCTRHC

SEQ ID NO: 473 - protein, 47 aa, Populus trichocarpus
RVCLSQSHHYKGPCLRGHNCAMVCKTEGFAGGECKGFISRCFCAKLC

SEQ ID NO: 474 - protein, 47 aa, Populus trichocarpus
RVCLSQSHSFKGPCVRGHNCASVCKTEGFPGGECKGFRRRCFCAKPC

SEQ ID NO: 475 - protein, 47 aa, Populus trichocarpus; identical to SEQ ID NO: 474

SEQ ID NO: 476 - protein, 47 aa, Populus trichocarpus
RVCQSQSHYFKGPCARDHNCAWVCRNEGFSGGRCKGFRRRCFCTKLC

SEQ ID NO: 477 - protein, 47 aa, Populus trichocarpus; identical to SEQ ID NO: 476

SEQ ID NO: 478 - protein, 47 aa, Prunus persica
RVCQSQSHGFRGPCIRHHNCALVCRNEGFSGGRCRGFRRRCFCTRLC

SEQ ID NO: 479 - protein, 47 aa, Prunus persica
RTCESQSNRFKGTCVSTSNCASVCQTEGFPGGHCRGFRRRCFCTKHC

SEQ ID NO: 480 - protein, 57 aa, Prunus persica
RDAKKVEPKKKTCDSQSQKFKGMCFLTSSCVQSCKSEGFTGGQCRGFRRRCFCSKNC

SEQ ID NO: 481 - protein, 57 aa, Prunus persica
RSAKKVEPKKRTCESQSQKFKGICFLSSNCATSCTTEGFNGGQCRGFRRRCFCSKAC

SEQ ID NO: 482 - protein, 57 aa, Prunus persica
KICEVTSKTFIGLCIKDRNCMVRCHSEGYGYGKCSHILRKCRCLKPCVAEETKENLP

SEQ ID NO: 483 - protein, 55 aa, Prunus persica
KVATKEESRTCESLSTKFKGFCLRSSRCAEACQKEGFMGGKCPGFRLRCTCTKKC

SEQ ID NO: 484 - protein, 55 aa, Prunus persica; identical to SEQ ID NO: 483

SEQ ID NO: 485 - protein, 55 aa, Prunus persica
KVETKETSRTCESLSTKFKGPCIRSSNCANICEEEGFKGGKCVGFRLRCTCTKNC

SEQ ID NO: 486 - protein, 55 aa, Prunus persica; identical to SEQ ID NO: 485

SEQ ID NO: 487 - protein, 55 aa, Prunus persica; identical to SEQ ID NO: 485

SEQ ID NO: 488 - protein, 55 aa, Prunus persica; identical to SEQ ID NO: 485

SEQ ID NO: 489 - protein, 55 aa, Prunus persica
KVETKETSRTCESLSTKFKGPCFRSSNCANICEKEGFKGGKCVGFRLRCTCTKNC

SEQ ID NO: 490 - protein, 55 aa, Prunus persica
KAATKENSRTCESLSTKFKGLCFRSSNCADICKKEGFLEGKCAGFRLRCTCTKKC

SEQ ID NO: 491 - protein, 47 aa, Pisum sativum
RDCESKSHKFKGTCLSDTNCASVCQTERFTGGHCRGFRHRCYCTTHC

SEQ ID NO: 492 - protein, 47 aa, Pisum sativum
RDCESPSQKFKGLCFSDTNCASVCQTEGFPGGKCEGLRHRCFCTKPC

SEQ ID NO: 493 - protein, 47 aa, Pisum sativum
RDCESPSQKFKGVCLSDRNCASVCQTEGFTGGDCKGLRQRCFCTKPC

SEQ ID NO: 494 - protein, 47 aa, Pisum sativum
RDCESPSQIFKGLCFTDTNCASVCHTEGFPGGKCKGLPKRCICTKPC

SEQ ID NO: 495 - protein, 46 aa, Pisum sativum
AGTCPSPSTKFKGVCVSDFDCMITCQMEGLSEGKCQSLKCICAKPC

SEQ ID NO: 496 - protein, 39 aa, Pisum sativum
SECNDEECEWSCIDKGFLDGVCSPSSNQCFCVIHRVIIR

SEQ ID NO: 497 - protein, 50 aa, Pisum sativum
NSCQSKSSQFKGICLSNKKCARVCHLEGFLEGGKCKGKLLKHCSCKKSIC

SEQ ID NO: 498 - protein, 45 aa, Pisum sativum
RGCKVKSTKFRGPCSFDKNCATVCRGEGYRDGDCHGLRRRCMCLC

SEQ ID NO: 499 - protein, 47 aa, Pisum sativum
RKCVSQSHRFHGPCLRDSNCATVCLGEGFTGGDCHGFRRRCFCSRFC

SEQ ID NO: 500 - protein, 56 aa, Pisum sativum
RHCESPSHRFKGICVRKSNCAAICQTEGFSSGKCRGFRSRCVCIKHCHHHEEEVQN

SEQ ID NO: 501 - protein, 57 aa, Pisum sativum
RLCESASHRFKGICVRKSNCGAVCQTEGFSGGKCRGLRARCMCTKHCHQEEEAEVKN

SEQ ID NO: 502 - protein, 57 aa, Pisum sativum
MRVDGARTNRMCDSKSHKFRGLCVSDTNCRNVCQTERFSDGHCRGARRRCYCQNPCI

SEQ ID NO: 503 - protein, 57 aa, Pisum sativum; identical to SEQ ID NO: 501

SEQ ID NO: 504 - protein, 47 aa, Quercus lobata
RTCESQSHRFKGPCVRKSNCASVCQTEGFHGGQCRGFRRRCFCTKHC

SEQ ID NO: 505 - protein, 47 aa, Quercus lobata
RVCESQSHKFKGACMSNHNCALVCRNEGFSGGRCRGFRHRCFCTRIC

SEQ ID NO: 506 - protein, 50 aa, Rosa chinensis
ENLRTCDSPSGKFKGPCIISSNCALTCQKEGFAGGHCRDFLRKCYCAKPC

SEQ ID NO: 507 - protein, 47 aa, Rosa chinensis
RTCETLSHKFKGTCLRESNCASVCQTEGFSGGDCRGLRRRCFCTKHC

SEQ ID NO: 508 - protein, 59 aa, Rosa chinensis
GIGPNVMVAEARTCESQSLKYEGMCLRESHCASVCQTEGYSGGDCHGLYSICVCTKDCQ

SEQ ID NO: 509 - protein, 58 aa, Rosa chinensis
GIGPNVTVAEARKCESQSHKFAGICLIESNCASICKTEGYSGGNCGGFRRRCFCTKDC

SEQ ID NO: 510 - protein, 47 aa, Rosa chinensis
RKCESQSHKFEGICLKQSNCASICKTEGYSGGNCRGFRRRCFCTKDC

SEQ ID NO: 511 - protein, 58 aa, Rosa chinensis
GIGPNVMVAEARKCESQSHKFAGICLIESNCASICKTEGYSGGNCGGFRRRCFCTKDC

SEQ ID NO: 512 - protein, 47 aa, Rosa chinensis
RVCEVPSKTFSGLCFSDTHCIVRCHSEGYGYGKCSHILRRCRCLKAC

SEQ ID NO: 513 - protein, 48 aa, Rosa chinensis
KICEVPSKTFKGLCVKDRNCVVRCHSEGYGNGKCSHVRRQCMCLKICD

SEQ ID NO: 514 - protein, 47 aa, Rosa chinensis
KICEYPSRTFKGLCVKDKNCVVRCRSEGYNYGKCSHVRRRCRCFKPC

SEQ ID NO: 515 - protein, 47 aa, Rosa chinensis; identical to SEQ ID NO: 284

SEQ ID NO: 516 - protein, 47 aa, Rosa chinensis
RVCESRSHGFHGTCIHHNNCALVCRNEGFPGGRCRGFRRRCFCTRNC

SEQ ID NO: 517 - protein, 47 aa, Salvia bowleyana
RTCESKSHRFKGICVRKANCAAVCQTEGFHSGHCRGFRRRCYCTRHC

SEQ ID NO: 518 - protein, 42 aa, Salvia bowleyana
RTCESESSSFKGLCLRNADCYIVCQTEGFSGGKCSCFCTKPC

SEQ ID NO: 519 - protein, 47 aa, Salvia bowleyana
RTCESESSTFKGLCLKNADCYIVCRTEGFSGGKCSSFLRRCFCTKPC

SEQ ID NO: 520 - protein, 74 aa, Salvia bowleyana
VVCSAASRLFSGLCFSDSNCSSICEKEGFLTGVCKGLQCICQKDCGGGDGGDPGQGGADQSALINRKLLDQLLV

SEQ ID NO: 521 - protein, 56 aa, Salvia bowleyana
AAASVVEEKRSCQSQSHKFKGKCFSDKNCGSVCQTEGFPSGKCHGLRQRCFCTRPC

SEQ ID NO: 522 - protein, 56 aa, Salvia bowleyana
AMCSAPSKLFRGVCLLRSNCSSICATEGFLYGLCKDLKCVCYKDCNVVGSGGDAQH

SEQ ID NO: 523 - protein, 74 aa, Salvia bowleyana
VMCSAASRLFSGLCFSDNNCSAICEKEGFLSGVCKGLKCVCQKDCNGGGGGGGNGGGDGGDQDQGPPAQGGGDQ

SEQ ID NO: 524 - protein, 74 aa, Salvia bowleyana
VMCSAASRLFSGLCFSDNNCSAICEKEGFLSGVCKGLKCVCQKDCNGGGGGNGGGDGGDQDQGPPAQGGGDQSP

SEQ ID NO: 525 - protein, 73 aa, Salvia bowleyana
AMCSAPSRLFRGLCFRRSNCTSICEKEGFLYGLCKGLKCVCYKDCNAAGGGGNGGDPGQGPPDQGGDDQPPPL

SEQ ID NO: 526 - protein, 56 aa, Salvia bowleyana; identical to SEQ ID NO: 521

SEQ ID NO: 527 - protein, 61 aa, Salvia bowleyana
AMCSAPSKLFRGVCLLRSNCSSICATEGFLYGLCKDLKCVCYKDCNVVGSGSGGSGGDAQH

SEQ ID NO: 528 - protein, 48 aa, Simmondsia chinensis
RICQSQSHLFRGPCVRNHNCASVCQNEGFSDGSCRGFRRRCFCAKFCP

SEQ ID NO: 529 - protein, 42 aa, Sechium edule
QSHGFHGACLSDHNCAVVCRNEGFSGGDCKGFRRRCFCTRHC

SEQ ID NO: 530 - protein, 48 aa, Sechium edule
RTCESPSRHFKGVCFSRNNCGHVCKTEGFHGGHCRGFRRRCFCTKHCL

SEQ ID NO: 531 - protein, 48 aa, Sechium edule
RTCESQSHHFKGLCFSKNNCGHVCKTEGFHGGHCRGFRRRCFCTKHCA

SEQ ID NO: 532 - protein, 50 aa, Sequoiadendron giganteum
RMCKSQSHTFKGYCVSDSNCKNVCRTEKFPTGGCDFHVASRKCYCYKPCA

SEQ ID NO: 533 - protein, 47 aa, Solanum lycopersicum
RTCESQSHRYKGPCVRKNNCANVCKTEGFSGGHCRGFRRRCFCAKHC

SEQ ID NO: 534 - protein, 47 aa, Solanum lycopersicum
RTCESQSHHFKGNCLSDTNCGSVCRTEGFTGGNCRGFRRRCFCTRNC

SEQ ID NO: 535 - protein, 50 aa, Solanum lycopersicum
AEARTCESQSNSFKGTCVRDSNCATVCQTEGFIGGNCRGFRRRCFCTRNC

SEQ ID NO: 536 - protein, 47 aa, Solanum lycopersicum
RTCEPQSHSFKGPCSRDSNCATVCQTEGFIGGDCRGLRRQCFCTTEC

SEQ ID NO: 537 - protein, 47 aa, Solanum lycopersicum
RTCESQSHRFKGPCVSEKNCASVCETEGFSGGDCRGFRRRCFCTRPC

SEQ ID NO: 538 - protein, 47 aa, Solanum lycopersicum
RVCMSPSHSYHGPCWHDHNCAIVCRNEGFSGGNCVGIQLKCYCTKLC

SEQ ID NO: 539 - protein, 47 aa, Solanum lycopersicum
RVCISQSHRYRGPCVLDHNCAIICRNEGFSGGDCIGWRRRCFCTKLC

SEQ ID NO: 540 - protein, 47 aa, Solanum pennellii
RTCETQSHKYKGPCVRKSNCANVCKTEGFRGGHCRGFRRRCFCTKNC

SEQ ID NO: 541 - protein, 55 aa, Solanum pennellii
GIRKTDAKLCQYHSETFHGVCVTGNNCNRHCQRENFEGGRCHGIRHYRCVCYRTC

SEQ ID NO: 542 - protein, 47 aa, Solanum pennellii
RHCESLSHRFKGPCASDKNCASICETERFSGGNCRGFRRRCFCTKPC

SEQ ID NO: 543 - protein, 47 aa, Solanum pennellii; identical to SEQ ID NO: 537

SEQ ID NO: 544 - protein, 47 aa, Solanum pennellii
RTCESQSHSFKGPCSRDSNCATVCQTEGFIGGDCRGLRRQCFCTTDC

SEQ ID NO: 545 - protein, 47 aa, Solanum pennellii
RTCESQSNSFKGTCVRDSNCATVCQTEGFIGGNCRGFRRRCFCTRNC

SEQ ID NO: 546 - protein, 47 aa, Solanum pennellii; identical to SEQ ID NO: 534

SEQ ID NO: 547 - protein, 47 aa, Solanum pennellii; identical to SEQ ID NO: 538

SEQ ID NO: 548 - protein, 47 aa, Solanum pennellii; identical to SEQ ID NO: 539

SEQ ID NO: 549 - protein, 47 aa, Schrenkiella parvula
RTCESKSHRFRGVCVSDGNCGNVCHNEGFGGGKCRGFRRRCFCTRHC

SEQ ID NO: 550 - protein, 47 aa, Schrenkiella parvula
RTCESQSHRFKGKCLSDGNCGNVCHNEGFGGGKCRGFRRRCFCTRNC

SEQ ID NO: 551 - protein, 45 aa, Schrenkiella parvula
KTCESPSGKYHGVCLNAKNCASVCPSEGFTGGHCKSLRCYCIKTC

SEQ ID NO: 552 - protein, 63 aa, Schrenkiella parvula
TEMGPADVEPRICETLSKVFKGMCWNQNNCAIMCREFEHFHSGHCRGFLRRCYCTKRCDGGPS

SEQ ID NO: 553 - protein, 52 aa, Tarenaya hassleriana
ERGEGRMCQSKSHHFHRTCVSNHNCAMVCRTEGFSGGHCVGFRRRCFCTRLC

SEQ ID NO: 554 - protein, 47 aa, Tarenaya hassleriana
RTCESPSHRFKGACLSETNCANVCHNEGFIGGNCRGLRRRCFCTKHC

SEQ ID NO: 555 - protein, 47 aa, Theobroma caccao
RTCQSQSHRFKGMCMRKSNCAAVCQTEGFHSGHCRGFHRRCFCTKHC

SEQ ID NO: 556 - protein, 47 aa, Theobroma caccao
RTCESKSHAFKGPCLSDHNCGLVCRNEGFPDGWCRGIRRRCFCTRRC

SEQ ID NO: 557 - protein, 47 aa, Theobroma caccao
RLCESKSHGFKGACLSDHNCGMVCSNEGFSGGRCRGFRHRCFCTRRC

SEQ ID NO: 558 - protein, 47 aa, Theobroma caccao
RVCESKSHKFKGPCLGDHNCALVCRNEGFSGGRCRGFRHRCFCTKLC

SEQ ID NO: 559 - protein, 47 aa, Thlaspi arvense
RTCESKSHRFKGACLSETNCKNVCHNEGFRGGNCRGLRRRCFCTRNC

SEQ ID NO: 560 - protein, 47 aa, Eutrema salsugineum
RKCESKSHRFKGKCLSETNCKNVCHNEGFTGGNCRGFRRRCFCTRNC

SEQ ID NO: 561 - protein, 46 aa, Eutrema salsugineum
RTCESASGKFQGVCLNSKSCASVCQSNEGFSGGHCNSLRCYCTKTC

SEQ ID NO: 562 - protein, 47 aa, Eutrema salsugineum
RMCQSKSHHFKHMCWSDHNCAIICRNEGFSGGRCRGFHRRCYCTRLC

SEQ ID NO: 563 - protein, 50 aa, Eutrema salsugineum
QQICKARSINFTALCLRWSKCKQACLTENFPDGRCIARRCFCMTPCHFLT

SEQ ID NO: 564 - protein, 52 aa, Eutrema salsugineum
QQICEARSINFRGMCMKWRNCQRVCMSEGFPDGRCAGFIRRCICRKPCPILS

SEQ ID NO: 565 - protein, 100 aa, Eutrema salsugineum
RMCESSSQLFNGPCLSTTNCANICHNEGFTDGDCQGFRRRCICTRPCTPRGMERKTLGLFFMLFLLLSADKYYFMDADVALKTAEARRCESKSHKFKGPC

SEQ ID NO: 566 - protein, 48 aa, Trifolium pratense
RTCESQSHKFKGPCASDHNCASVCQTERFPGGHCRGFRRRCFCTTHCS

SEQ ID NO: 567 - protein, 55 aa, Trifolium pratense
EMVVETEGRHCESKSHRFKGMCLSDHNCASVCHVEGFPGGNCRGFRRRCFCKKRC

SEQ ID NO: 568 - protein, 48 aa, Trifolium pratense
RKCESKRRWIYGNCLSDEKCASDCLVKGFLFGKCRGYDNRCYCKKPRC

SEQ ID NO: 569 - protein, 47 aa, Tripterygium wilfordii
RVCASKSHYYKGPCLHNHNCAMVCRNEGFSGGKCHGLGRRCFCTKLC

SEQ ID NO: 570 - protein, 47 aa, Tripterygium wilfordii
RVCLSPSHKFHGTCVRGHNCAEICRTEGFSGGDCHGFRRRCMCSRRC

SEQ ID NO: 571 - protein, 57 aa, Phtheirospermum japonicum
AGEEAVGEEKKACKYKSEKFWGICITDSKCIEVCRREGFQTGDCKGLRRRCYCVNNC

SEQ ID NO: 572 - protein, 82 aa, Vigna mungo
VASGGKDKTKKPGRYIATELENIETNYFSDYTGFEIQIDSTDVDDAIDTLSEVASKKLVQILLRFVALFLPVAPIPPLPIPS

SEQ ID NO: 573 - protein, 48 aa, Zea mays
RTCQSQSHRFRGPCLRRSNCANVCRTEGFPGGRCRGFRRRCFCTTHCH

SEQ ID NO: 574 - protein, 55 aa, Zea mays
RVVVEETLCLSQSHAFKGVCLSNTNCDNVCKTEKFTGGECKMDGVMRKCYCKKVC

SEQ ID NO: 575 - protein, 49 aa, Zea mays
RHCLSQSHRFKGLCMSSNNCANVCQTENFPGGECKAEGATRKCFCKKIC

SEQ ID NO: 576 - protein, 55 aa, Zea mays
GPVTMVSARKCESQSFRFKGPCSRDANCANVCLTEGFTGGVCKGLRHRCFCTRDC

SEQ ID NO: 577 - protein, 22 aa, Artificial
  synthetic
  MOD_RES (22)..(22): AMIDATION
GACLKNRHSKHYGCYCYYRHCY

SEQ ID NO: 578 - protein, 22 aa, Artificial
  synthetic
  MOD_RES (22)..(22): AMIDATION
GACLKNRHSKHYGWFWYYRHCY

SEQ ID NO: 579 - protein, 22 aa, Artificial
  synthetic
  MOD_RES (22)..(22): AMIDATION
GACLKNRHSKHYGFFWYYRHCY

SEQ ID NO: 580 - protein, 16 aa, Artificial
  synthetic
  MOD_RES (16)..(16): AMIDATION
GRCRGFRRRCFCTTHC

SEQ ID NO: 581 - protein, 17 aa, Artificial
  synthetic
  MOD_RES (17)..(17): AMIDATION
GGRCKGFLRRFWFTRIC

SEQ ID NO: 582 - protein, 17 aa, Artificial
  synthetic
  MOD_RES (17)..(17): AMIDATION
GGRCKGFRRRWYWTRIC

SEQ ID NO: 583 - protein, 16 aa, Artificial
  synthetic
  MOD_RES (16)..(16): AMIDATION
SGRCRILFRCFCTKNC

SEQ ID NO: 584 - protein, 16 aa, Artificial
  synthetic
  MOD_RES (16)..(16): AMIDATION
SGRCRILFRWFWTKNC

SEQ ID NO: 585 - protein, 16 aa, Artificial
  synthetic
  MOD_RES (16)..(16): AMIDATION
SGRCRILFRWYFTKNC

SEQ ID NO: 586 - protein, 18 aa, Artificial
  synthetic
  MOD_RES (18)..(18): AMIDATION
GACHRQGFGFACFCYKKC

SEQ ID NO: 587 - protein, 16 aa, Artificial
  synthetic
  VARIANT (2)..(2): Xaa is any amino acid
  VARIANT (4)..(13): Xaa is any amino acid, Xaa7 to Xaa13 are optionally present
  VARIANT (14)..(16): Xaa is F, W, or Y
GXCXXXXXXXXXXXXX

SEQ ID NO: 588 - protein, 18 aa, Artificial
  synthetic
  VARIANT (2)..(2): Xaa is any amino acid
  VARIANT (4)..(15): Xaa is any amino acid, Xaa7 to Xaa15 are optionally present
  VARIANT (16)..(18): Xaa is F, W, or Y
GXCXXXXXXXXXXXXXXX

SEQ ID NO: 589 - protein, 21 aa, Artificial
  synthetic
  VARIANT (2)..(2): Xaa is any amino acid
  VARIANT (4)..(18): Xaa is any amino acid, Xaa7 to Xaa18 are optionally present
  VARIANT (19)..(21): Xaa is F, W, or Y
GXCXXXXXXXXXXXXXXXXXX

SEQ ID NO: 590 - protein, 16 aa, Artificial
  synthetic
  VARIANT (2)..(2): Xaa is any amino acid
  VARIANT (4)..(15): Xaa is any amino acid, Xaa7 to Xaa15 are optionally present
GXCXXXXXXXXXXXXC

SEQ ID NO: 591 - protein, 19 aa, Artificial
  synthetic
  VARIANT (2)..(2): Xaa is any amino acid
  VARIANT (4)..(18): Xaa is any amino acid, Xaa7 to Xaa18 are optionally present
GXCXXXXXXXXXXXXXXXC